(19) United States
(12) Patent Application Publication
(10) Pub. No.: US 2004/0147581 A1
(43) Pub. Date: Jul. 29, 2004

(54) Method of using a Cox-2 inhibitor and a 5-HT1A receptor modulator as a combination therapy

(75) Inventors: Duncan P. Taylor, Kalamazoo, MI (US); Diane T. Stephenson, Portage, MI (US)

Correspondence Address: Charles E. Dunlap, P.O. Box 11070, Columbia, SC, 29211-1070, US

(73) Assignee: Pharmacia Corporation, St. Louis, MO (US)

(21) Appl. No.: 10/702,403

(22) Filed: Nov. 5, 2003

Related U.S. Application Data
(60) Provisional application No. 60/427,198, filed on Nov. 18, 2002.

(51) Int. Cl. A61K 31/60; A61K 31/415; A61K 31/192; A61K 31/21

(52) U.S. Cl. 514/406; 514/570; 514/569; 514/509; 514/165

(57) ABSTRACT

Compositions and methods to treat or prevent pain, inflammation, or inflammation-related disorder, as well as a neurologic disorder involving neurodegrneration in a subject that is in need of such prevention or treatment involve a combination of a Cox-2 inhibitor and a 5-HT_{1A} receptor modulator.

### CROSS-RELEFERENCE TO RELATED PATENTS AND PATENT APPLICATIONS

This application is a non-provisional of U.S. Provisional Patent Application No. 60/427,198, filed Nov. 18, 2002, which is incorporated herein by reference in its entirety.

### BACKGROUND OF THE INVENTION

(1) Field of the Invention

The present invention relates to compositions and methods for the treatment or prevention of pain, inflammation, or inflammation-related disorder in a mammal using a combination of a Cox-2 inhibitor and a 5-HT_{1A} receptor modulator.

(2) Description of Related Art

Serotonin (5-hydroxytryptamine, or 5-HT) is involved in the origin of many disease states. Recently, at least fourteen different 5-HT receptor subtypes have been identified and characterized (“A Review of Central 5-HT Receptors and Their Function,” N. M. Barnes and T. Sharp, Neuropharmacology, 38:1083-1152 (1999)). The 5-HT₁ receptor family consists of five receptor subtypes: 5-HT_{1A}, 5-HT_{1B}, 5-HT_{1D}, 5-HT_{1E}, and 5-HT_{1F}. The 5-HT_{1A} receptor is the best known among the different 5-HT receptors and is widely distributed in the central nervous system (L. Lanfurney and M. Hamon, Nuclear Medicine & Biology, 27:429-435 (2000)).

Studies on the 5-HT_{1A} receptor have shown potential roles in a variety of physiological processes including, neuroendocrine function, thermoregulation, vasoreactive headaches, sexual behavior, food intake, tooth-germ morphogenesis, immune function, aggression, depression and anxiety (J. R. Raymond, et al., Br. J. Pharmacol., 127:1751-1764 (1999)). Other studies have shown the potential use of 5-HT_{1A} agonists in glaucoma to lower intraocular pressure in the eye (N. N. Osborne, et al., Eye, 14:454-463 (2000)). Recent studies have shown the involvement of 5-HT_{1A} receptors in the transmission of nociceptive (pain) information in the spinal cord resulting from nerve injury or inflammation (Z.-Y. Liu, et al, Neuroscience, 112(2):399-407 (2002)). Growing evidence suggests that the 5-HT_{1A} receptor is important in learning and memory processes (A. Meneses, Neurosci. Biobehav. Rev., 23:1111-1125 (1999)) and that 5-HT_{1A} receptor antagonists may have utility in treating cognitive dysfunction associated with Alzheimer's disease (L. E. Schechter, et al., Curr. Pharm. Des., 8(2):139-145 (2002)). A 5-HT_{1A} receptor agonist has shown a neuroprotective effect associated with its ability to inhibit ischemia-induced release of glutamate in the brain in a stroke model (I. Semkova, et al., Eur. J. Pharmacol., 359:251-260 (1998)).

Prostaglandins play a major role in the inflammation process and the inhibition of prostaglandin production, especially production of PGG₂, PGH₂ and PGE₂, has been a common target of antiinflammatory drug discovery. However, common non-steroidal antiinflammatory drugs (NSAIDs) that are active in reducing the prostaglandin-induced pain and swelling associated with the inflammation process are also active in affecting other prostaglandin-regulated processes not associated with the inflammation process. Thus, use of high doses of most common NSAIDs can produce severe side effects, including life threatening ulcers, that limit their therapeutic potential. Previous NSAIDs have been found to prevent the production of prostaglandins by inhibiting enzymes in the human arachidonic acid/prostaglandin pathway, including the enzyme cyclooxygenase (Cox). The recent discovery of an inducible enzyme associated with inflammation (named “cyclooxygenase-2 (Cox-2)” or “prostaglandin G/H synthase II”) provides a viable target of inhibition which more effectively reduces inflammation and produces fewer and less drastic side effects.

Compounds that selectively inhibit the cyclooxygenase-2 enzyme have been discovered. These compounds selectively inhibit the activity of Cox-2 to a greater extent than the activity of Cox-1. The Cox-2-selective inhibitors are believed to offer advantages that include the capacity to prevent or reduce inflammation while avoiding harmful side effects associated with the inhibition of Cox-1. Thus, cyclooxygenase-2-selective inhibitors have shown great promise for use in therapies—especially in therapies that require extended administration, such as for pain and inflammation control for arthritis. Additional information on the identification of cyclooxygenase-2-selective inhibitors can be found in: (1) Buttgereit, F. et al., Am. J. Med., 110(3 Suppl. 1):13-9 (2001); (2) Osiri, M. et al, Arthritis Care Res., 12(5):351-62 (1999); (3) Buttar, N. S. et al., Mayo Clin. Proc., 75(10):1027-38 (2000); (4) Wollheim, F. A., Current Opin. Rheumatol., 13:193-201 (2001); (5) U.S. Pat. No. 5,434,178 (1,3,5-trisubstituted pyrazole compounds); (6) U.S. Pat. No. 5,476,944 (derivatives of cyclic phenolic thioethers); (7) U.S. Pat. No. 5,643,933 (substituted sulfonylphenylheterocycles); U.S. Pat. No. 5,859,257 (isoxazole compounds); (8) U.S. Pat. No. 5,932,598 (prodrugs of benzenesulfonamide-containing Cox-2 inhibitors); (9) U.S. Pat. No. 6,156,781 (substituted pyrazolyl benzenesulfonamides); and (10) U.S. Pat. No. 6,110,960 (for dihydrobenzopyran and related compounds).

Cox-2 inhibitors have also been described for the treatment of cancer (WO98/16227) and for the treatment of tumors (See, EP 927,555, and Rozic et al., Int. J. Cancer, 93(4):497-506 (2001)). Celecoxib, a selective inhibitor of Cox-2, exerted a potent inhibition of fibroblast growth factor-induced corneal angiogenesis in rats. (Masferrer et al., Proc. Am. Assoc. Cancer Research 1999, 40: 396). WO 98/41511 describes 5-(4-sulphonyl-phenyl)-pyridazinone derivatives used for treating cancer. WO 98/41516 describes (methylsulphonyl)phenyl-2-(5H)-furanone derivatives that can be used in the treatment of cancer. Kalgutkar, A. S. et al., Curr. Drug Targets, 2(1):79-106 (2001) suggest that Cox-2 selective inhibitors could be used to prevent or treat cancer by affecting tumor viability, growth, and metastasis. Masferrer et al., in Ann. NY Acad. Sci., 889:84-86 (1999) describe Cox-2 selective inhibitors as antiangiogenic agents with potential therapeutic utility in several types of cancers. The utility of Cox-2 inhibition in clinical cancer prevention was described by Lynch, P. M., in Oncology, 15(3):21-26 (2001), and Watanabe et al., in Biofactors 2000, 12(1-4):129-133 (2000) described the potential of Cox-2 selective inhibitors for chemopreventive agents against colon cancer.

Additionally, various combination therapies using Cox-2 inhibitors with other selected combination regimens for the treatment of cancer have also been reported. See e.g., FR 27 71 005 (compositions containing a cyclooxygenase-2 inhibitor and N-methyl-d-aspartate (NMDA) antagonist used to treat cancer and other diseases); WO 99/18960 (combination comprising a cyclooxygenase-2 inhibitor and an inducible nitric-oxide synthase inhibitor (iNOS) that can be used to treat colorectal and breast cancer); WO 99/13799 (combination of a cyclooxygenase-2 inhibitor and an opioid analgesic); WO 97/36497 (combination comprising a cyclooxygenase-2 inhibitor and a 5-lipoxygenase inhibitor useful in treating cancer); WO 97/29776 (composition comprising a cyclooxygenase-2 inhibitor in combination with a leukotriene B4 receptor antagonist and an immunosuppressive drug); WO 97/29775 (use of a cyclooxygenase-2 inhibitor in combination with a leukotriene A4 hydrolase inhibitor and an immunosuppressive drug); WO 97/29774 (combination of a cyclooxygenase-2 inhibitor and prostagladin or antiulcer agent useful in treating cancer); WO 97/11701 (combination comprising of a cyclooxygenase-2 inhibitor and a leukotriene B receptor antagonist useful in treating colorectal cancer); WO 96/41645 (combination comprising a cyclooxygenase-2 inhibitor and leukotriene A hydrolase inhibitor); WO 96/03385 (3,4,-Di substituted pyrazole compounds given alone or in combination with NSAIDs, steroids, 5-LO inhibitors, LTB4 antagonists, or LTA4 hydrolase inhibitors for the treatment of cancer); WO 98/47890 (substituted benzopyran derivatives that may be used alone or in combination with other active principles); WO 00/38730 (method of using cyclooxygenase-2 inhibitor and one or more antineoplastic agents as a combination therapy in the treatment of neoplasia); Mann, M. et al., Gastroenterology, 120(7):1713-1719 (2001) (combination treatment with Cox-2 and HER-2/neu inhibitors reduced colorectal carcinoma growth).

Other reports have indicated the Cox-2 selective inhibitors have cardiovascular applications. For example, Saito, T. et al., in Biochem. Biophys. Res. Comm., 273:772-775 (2000), reported that the inhibition of Cox-2 improves cardiac function in myocardial infarction. Ridker, P. M. et al., in The New England J. of Med., 336(14):973-979 (1997), raised the possibility that anti-inflammatory agents may have clinical benefits in preventing cardiovascular disease. In addition, Cox-2 selective inhibitors have been proposed for therapeutic use in cardiovascular disease when combined with modulation of inducible nitric oxide synthase (See, Baker, C. S. R. et al., Arterioscler. Thromb. Vasc. Biol., 19:646-655 (1999)), and with HMG-CoA reductase inhibitor (U.S. Pat. No. 6,245,797).

Recent studies have shown that Cox-2 and its reaction products participate in ischemic injury in the human brain caused by stroke or other injury (C. Iadecola, et al., Proc. Natl. Acad. Sci. U.S.A., 98(3):1294-1299 (2001)). A selective Cox-2 inhibitor has been shown to be neuroprotective, resulting in improvements in behavorial deficits caused by spinal cord ischemia (P. A. Lapchak, et al., Stroke, 32:1220-1225 (2001)). Studies have shown that Cox-2 expression is elevated in Alzheimer's disease brains, is correlated with dementia, and causes detrimental alterations of the neuronal cell cycle (Xiang et al., Neurobiol. Aging, 23:327-334 (2002)).

EP 1064967 describes the combination of 5-HT_{1A} receptor agonists, caffeine, and either a Cox-2 inhibitor or NSAID for the treatment of migraine.

EP 1064966 describes the combination of a 5-HT_{1A} receptor agonist, caffeine, and a Cox-2 inhibitor for the treatment of migraine.

EP 1064948 describes the combination of a 5-HT_{1A} receptor antagonist, caffeine, and a Cox-2 inhibitor for the treatment of migraine.

EP 1051995 describes the combination of 5-HT_{1A} receptor agonists and either a Cox-2 inhibitor or NSAID for the treatment of migraine.

EP 1051994 describes the combination of a 5-HT_{1A} agonist and a Cox-2 inhibitor for the treatment of migraine.

EP 1051993 describes the combination of 5-HT_{1A} receptor agonists and either a Cox-2 inhibitor or NSAID for the treatment of migraine.

US 20020077328 describes the combination of selective Cox-2 inhibitors and vasomodulator compounds for generalized pain and headache pain.

WO 0048583 describes the combination of 5-HT agonists with Cox-2 inhibitors for the treatment of migraine.

U.S. Pat. Nos. 6,420,432, 6,413,961, 6,261,279, 6,254,585, 6,242,447, 6,210,394, 6,056,715, 5,860,950, 5,858,017, 5,820,583, and 5,800,385 describe various types of irrigation solution and a method for inhibition of pain and inflammation, where the solutions can contain a Cox-2 inhibitor and some type of serotonin agonist or 5-HT_{1A} receptor agonist.

In U.S. Patent Publication No. 2002/0077328 A1, Hassan et al. disclose, among other things, a method for treatment of headache symptoms by administering a selective Cox-2 inhibitor and a vasomodulator, where the IC₅₀ of the combination for binding of 5TH_{1A} [HT_{1A}] receptors is at least about 250 nM.

A need remains, however, for an improved method of treating and preventing pain, inflammation or inflammation-related disorders, and also for treating and preventing neorologic disorders involving neurodegeneration. In particular, it would be useful to provide such a method by utilizing a combination of therapeutic agents that is more efficacious and safer that presently available methods.

### SUMMARY OF THE INVENTION

Briefly, therefore, the present invention is directed to a novel composition comprising a Cox-2 inhibitor and a 5-HT_{1A} receptor modulator.

The present invention is also directed to a novel method for the treatment or prevention of pain, inflammation, or inflammation-related disorder in a mammal in need thereof, comprising administering to the mammal a Cox-2 inhibitor and a 5-HT_{1A} receptor modulator.

The present invention is also directed to a novel pharmaceutical composition for the treatment or prevention of pain, inflammation, or inflammation-related disorder, the pharmaceutical composition comprising a Cox-2 inhibitor, a 5-HT_{1A} receptor modulator, and a pharmaceutically-acceptable excipient.

The present invention is also directed to a novel kit that is suitable for use in the treatment or prevention of pain, inflammation, or inflammation-related disorder wherein the kit comprises a first dosage form comprising a Cox-2 inhibitor and a second dosage form comprising a 5-HT_{1A} receptor modulator, in quantities which comprise a therapeutically effective amount of the compounds for the treatment or prevention of pain, inflammation, or inflammation-related disorder.

The present invention is also directed to a novel method for the treatment or prevention of neurologic disease involving neurodegeneration in a mammal in need thereof, comprising administering to the mammal a Cox-2 inhibitor and a 5-HT_{1A} receptor modulator.

Among the several advantages found to be achieved by the present invention, therefore, may be noted the provision of an improved method of treating or preventing pain, inflammation or inflammation-related disorders, and treatment or prevention of neurologic diseases involving neurodegeneration, the provision of such a method by utilizing a combination of therapeutic agents that is more efficacious and safer than methods and compositions that are presently available, and the provision of therapeutic combinations and methods for the prevention and treatment of pain, inflammation and inflammation-related disorders.

### DETAILED DESCRIPTION OF THE INVENTION

In accordance with the present invention, it has been discovered that pain, inflammation, or inflammation-related disorders in a subject—in particular, a mammal—can be treated or prevented by a combination therapy method that involves administering to the subject an amount of a Cox-2 inhibitor and an amount of a 5-HT_{1A} receptor modulator. In preferred embodiments, the amount of the Cox-2 inhibitor and the amount of the 5-HT_{1A} receptor modulator together comprise a therapeutically effective amount for the treatment or prevention of pain, inflammation or inflammation-related disorder in the subject.

Also disclosed herein is a composition comprising an amount of a Cox-2 inhibitor and an amount of a 5-HT_{1A} receptor modulator wherein the amount of the Cox-2 inhibitor and the amount of the 5-HT_{1A} receptor modulator together comprise a therapeutically effective amount for the treatment or prevention of pain, inflammation or inflammation-related disorder.

A component of the present invention is a Cox-2 inhibitor. The terms “cyclooxygenase-2 inhibitor”, or “Cox-2 inhibitor”, which can be used interchangeably herein, embrace compounds which inhibit the Cox-2 enzyme regardless of the degree of inhibition of the Cox-1 enzyme, and include pharmaceutically acceptable salts of those compounds. Thus, for purposes of the present invention, a compound is considered a Cox-2 inhibitor irrespective of whether the compound inhibits the Cox-2 enzyme to an equal, greater, or lesser degree than the Cox-1 enzyme.

In one embodiment of the present invention, it is preferred that the Cox-2 inhibitor compound is a non-steroidal anti-inflammatory drug (NSAID). Therefore, preferred materials that can serve as the Cox-2 inhibitor of the present invention include non-steroidal anti-inflammatory drug compounds, a pharmaceutically acceptable salt thereof, or a pure (−) or (+) optical isomeric form thereof.

Examples of NSAID compounds that are useful in the present invention include acemetacin, acetyl salicylic acid, alclofenac, alminoprofen, azapropazone, benorylate, benoxaprofen, bucloxic acid, carprofen, choline magnesium trisalicylate, clidanac, clopinac, dapsone, diclofenac, diflunisal, droxicam, etodolac, fenoprofen, fenbufen, fenclofenec, fentiazac, floctafenine, flufenisal, flurbiprofen, (r)-flurbiprofen, (s)-flurbiprofen, furofenac, feprazone, flufenamic acid, fluprofen, ibufenac, ibuprofen, indometacin, indomethacin, indoprofen, isoxepac, isoxicam, ketoprofen, ketorolac, miroprofen, piroxicam, meloxicam, mefenamic, mefenamic acid, meclofenamic acid, meclofen, nabumetone, naproxen, niflumic acid, oxaprozin, oxipinac, oxyphenbutazone, phenylbutazone, podophyllotoxin derivatives, proglumetacin, piprofen, pirprofen, prapoprofen, salicylic acid, salicylate, sudoxicam, suprofen, sulindac, tenoxicam, tiaprofenic acid, tiopinac, tioxaprofen, tolfenamic acid, tolmetin, zidometacin, zomepirac, and 2-fluoro-a-methyl[1,1′-biphenyl]-4-acetic acid, 4-(nitrooxy)butyl ester.

In a preferred embodiment, the Cox-2 inhibitor is a Cox-2 selective inhibitor. The term “Cox-2 selective inhibitor” embraces compounds which selectively inhibit the Cox-2 enzyme over the Cox-1 enzyme, and also include pharmaceutically acceptable salts and prodrugs of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase activity. Preferred Cox-2 selective inhibitors of the present invention have a Cox-2 IC₅₀ of less than about 1 μM, more preferred of less than about 0.5 μM, and even more preferred of less than about 0.2 μM.

Preferred Cox-2 selective inhibitors have a Cox-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of Cox-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic Cox-2 selective inhibitor valdecoxib. An example of a preferred Cox-2 selective inhibitor prodrug is sodium parecoxib. A class of prodrugs of Cox-2 inhibitors is described in U.S. Pat. No. 5,932,598.

The Cox-2 selective inhibitor of the present invention can be, for example, the Cox-2 selective inhibitor meloxicam, Formula B-1 (CAS registry number 71125-38-7), or a pharmaceutically acceptable salt or prodrug thereof.

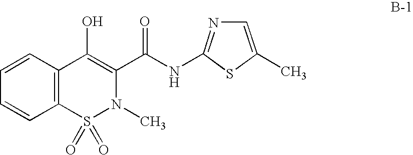

In another embodiment of the invention the Cox-2 selective inhibitor can be the Cox-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

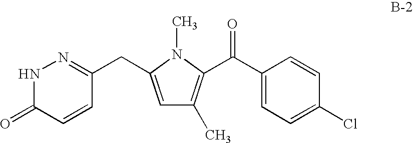

As used herein, the term “alkyl”, either alone or within other terms such as “haloalkyl” and “alkylsulfonyl”; embraces linear or branched radicals having one to about twenty carbon atoms. Lower alkyl radicals have one to about ten carbon atoms. The number of carbon atoms can also be expressed as “C₁-C₅”, for example. Examples of lower alkyl radicals include methyl, ethyl, n-propyl, isopropyl, n-butyl, isobutyl, sec-butyl, tert-butyl, pentyl, isoamyl, hexyl, octyl and the, like.

The term “alkenyl” refers to an unsaturated, acyclic hydrocarbon radical, linear or branched, in so much as it contains at least one double bond. The alkenyl radicals may be optionally substituted with groups such as those defined below. Examples of suitable alkenyl radicals include propenyl, 2-chloropropylenyl, buten-1yl, isobutenyl, penten-1yl, 2-methylbuten-1-yl, 3-methylbuten-1-yl, hexen-1-yl, 3-hydroxyhexen-1-yl, hepten-1-yl, octen-1-yl, and the like.

The term “alkynyl” refers to an unsaturated, acyclic hydrocarbon radical, linear or branched, in so much as it contains one or more triple bonds, such radicals preferably containing 2 to about 6 carbon atoms, more preferably from 2 to about 3 carbon atoms. The alkynyl radicals may be optionally substituted with groups such as described below. Examples of suitable alkynyl radicals include ethynyl, proynyl, hydroxypropynyl, butyn-1-yl, butyn-2-yl, pentyn-1-yl, pentyn-2-yl, 4-methoxypentyn-2-yl, 3-methylbutyn-1-yl, hexyl-1-yl, hexyn-2-yl, hexyn-3-yl, 3,3-dimethylbutyn-1-yl radicals, and the like.

The term “oxo” means a single double-bonded oxygen.

The terms “hydrido”, “—H”, or “hydrogen”, denote a single hydrogen atom (H). This hydrido radical may be attached, for example, to an oxygen atom to form a hydroxyl radical, or two hydrido radicals may be attached to a carbon atom to form a methylene (—CH₂—) radical.

The term “halo” means halogens such as fluorine, chlorine, and bromine or iodine atoms. The term “haloalkyl” embraces radicals wherein any one or more of the alkyl carbon atoms is substituted with halo as defined above. Specifically embraced are monohaloalkyl, dihaloalkyl, and polyhaloalkyl radicals. A monohaloalkyl radical, for one example, may have a bromo, chloro, or a fluoro atom within the radical. Dihalo alkyl radicals may have two or more of the same halo atoms or a combination of different halo radicals and polyhaloalkyl radicals may have more than two of the same halo atoms or a combination of different halo radicals.

The term “hydroxyalkyl” embraces linear or branched alkyl radicals having one to about ten carbon atoms any one of which may be substituted with one or more hydroxyl radicals.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals. The “alkoxy” or “alkoxyalkyl” radicals may be further substituted with one or more halo atoms, such as fluoro, chloro, or bromo, to provide “haloalkoxy” or “haloalkoxyalkyl” radicals. Examples of “alkoxy” radicals include methoxy, butoxy, and trifluoromethoxy.

The term “aryl”, whether used alone or with other terms, means a carbocyclic aromatic system containing one, two, or three rings wherein such rings may be attached together in a pendent manner, or may be fused. The term “aryl” embraces aromatic radicals such as phenyl, naphthyl, tetrahydronapthyl, indane, and biphenyl. The term “heterocyclyl” means a saturated or unsaturated mono- or multi-ring carbocycle wherein one or more carbon atoms are replaced by N, S, P, or O. This includes, for example, structures such as:

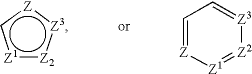

where Z, Z¹, Z², or Z³ is C, S, P, O, or N, with the proviso that one of Z, Z¹, Z², or Z³ is other than carbon, but is not O or S when attached to another Z atom by a double bond or when attached to another O or S atom. Furthermore, the optional substituents are understood to be attached to Z, Z¹, Z², or Z³ only when each is C. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others.

The term “heteroaryl” embraces unsaturated heterocyclic radicals. Examples of unsaturated heterocyclic radicals include thienyl, pyrryl, furyl, pyridyl, pyrimidyl, pyrazinyl, pyrazolyl, oxazolyl, isoxazolyl, imidazolyl, thiazolyl, pyranyl, and tetrazolyl. The term also embraces radicals where heterocyclic radicals are fused with aryl radicals. Examples of such fused bicyclic radicals include benzofuran, benzothiophene, and the like.

The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical. The term “aminosulfonyl” denotes a sulfonyl radical substituted with an amine radical, forming a sulfonamide (—SO₂—NH₂).

The terms “carboxy” or “carboxyl”, whether used alone or with other terms, such as “carboxyalkyl”, denotes —CO₂—H. The term “carboxyalkyl” embraces radicals having a carboxyradical as defined above, attached to an alkyl radical. The term “carbonyl”, whether used alone or with other terms, such as “alkylcarbonyl”, denotes —(C═O)—. The term “alkylcarbonyl” embraces radicals having a carbonyl radical substituted with an alkyl radical. An example of an “alkylcarbonyl” radical is CH₃— (CO)—. The term “alkoxycarbonyl” means a radical containing an alkoxy radical, as defined above, attached via an oxygen atom to a carbonyl (C═O) radical. Examples of such “alkoxycarbonyl” radicals include (CH₃)₃—C—O—C═O)— and —(O═)C—OCH₃. The term “amino”, whether used alone or with other terms, such as “aminocarbonyl”, denotes —NH₂.

The term “heterocycloalkyl” embraces heterocyclic-substituted alkyl radicals such as pyridylmethyl and thienylmethyl. The terms “aralkyl”, or “arylalkyl” embrace aryl-substituted alkyl radicals such as benzyl, diphenylmethyl, triphenylmethyl, phenylethyl, and diphenylethyl. The terms benzyl and phenylmethyl are interchangeable. The term “cycloalkyl” embraces radicals having three to ten carbon atoms, such as cyclopropyl cyclobutyl, cyclopentyl, cyclohexyl, and cycloheptyl. The term “cycloalkenyl” embraces unsaturated radicals having three to ten carbon atoms, such as cylopropenyl, cyclobutenyl, cyclopentenyl, cyclohexenyl, and cycloheptenyl.

The term “alkylthio” embraces radicals containing a linear or branched alkyl radical, of one to ten carbon atoms, attached to a divalent sulfur atom. An example of “alkylthio” is methylthio, (CH₃—S—). The term “alkylsulfinyl” embraces radicals containing a linear or branched alkyl radical, of one to ten carbon atoms, attached to a divalent —S(—O)— atom. The term “acyl”, whether used alone, or within a term such as “acylamino”, denotes a radical provided by the residue after removal of hydroxyl from an organic acid.

The term “cyano”, used either alone or with other terms, such as “cyanoalkyl”, refers to C≡N. The term “nitro” denotes —NO₂.

In one embodiment of the invention the Cox-2 selective inhibitor is of the chromene/chroman structural class, which encompasses substituted benzopyrans or substituted benzopyran analogs, as well as substituted benzothiopyrans, dihydroquinolines, or dihydronaphthalenes having the structure of any one of the general Formulas I, II, III, IV, V, and VI, shown below, and including, by way of non-limiting example, the structures disclosed in Table 1, and the diastereomers, enantiomers, racemates, tautomers, salts, esters, amides and prodrugs thereof.

Benzopyrans that can serve as a Cox-2 selective inhibitor of the present invention include substituted benzopyran derivatives that are described in U.S. Pat. Nos. 6,271,253 and 6,492,390. One such class of compounds is defined by the general formula shown below in formula I:

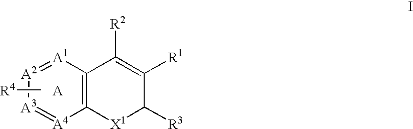

wherein X¹ is selected from O, S, CR^{c}R^{b} and NR^{a};

wherein R^{a} is selected from hydrido, C₁-C₃-alkyl, (optionally substituted phenyl)-C₁-C₃-alkyl, acyl and carboxy-C₁-C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b}R^{c} forms a 3-6 membered cycloalkyl ring;

wherein R¹ is selected from carboxyl, aminocarbonyl, C₁-C₆-alkylsulfonylaminocarbonyl and C₁-C₆-alkoxycarbonyl;

wherein R² is selected from hydrido, phenyl, thienyl, C₁-C₆-alkyl and C₂-C₆-alkenyl;

wherein R³ is selected from C₁-C₃-perfluoroalkyl, chloro, C₁-C₆-alkylthio, C₁-C₆-alkoxy, nitro, cyano and cyano-C₁-C₃-alkyl;

wherein R⁴ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₁-alkoxycarbonyl, formyl, C₁-C₆-haloalkylcarbonyl and C₁-C₆-alkylcarbonyl; and

wherein the A ring atoms A¹, A², A³ and A⁴ are independently selected from carbon and nitrogen with the proviso that at least two of A¹, A², A³ and A⁴ are carbon;

or wherein R⁴ together with ring A forms a radical selected from naphthyl, quinolyl, isoquinolyl, quinolizinyl, quinoxalinyl and dibenzofuryl; or an isomer or pharmaceutically acceptable salt thereof.

Another class of benzopyran derivatives that can serve as the Cox-2 selective inhibitor of the present invention includes compounds having the structure of formula II:

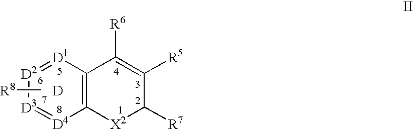

wherein X² is selected from O, S, CR^{c}R^{b} and NR^{a};

wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{c}R^{b} form a cyclopropyl ring;

wherein R⁵ is selected from carboxyl, aminocarbonyl, C₁-C₆-alkylsulfonylaminocarbonyl and C₁-C₆-alkoxycarbonyl;

wherein R⁶ is selected from hydrido, phenyl, thienyl, C₂-C₆-alkynyl and C₂-C₆-alkenyl;

wherein R⁷ is selected from C₁-C₃-perfluoroalkyl, chloro, C₁-C₆-alkylthio, C₁-C₆-alkoxy, nitro, cyano and cyano-C₁-C₃-alkyl;

wherein R⁸ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and

wherein the D ring atoms D¹, D², D³ and D⁴ are independently selected from carbon and nitrogen with the proviso that at least two of D¹, D², D³ and D⁴ are carbon; or

wherein R⁸ together with ring D forms a radical selected from naphthyl, quinolyl, isoquinolyl, quinolizinyl, quinoxalinyl and dibenzofuryl; or an isomer or pharmaceutically acceptable salt thereof.

Other benzopyran Cox-2 selective inhibitors useful in the practice of the present invention are described in U.S. Pat. Nos. 6,034,256 and 6,077,850. The general formula for these compounds is shown in formula III:

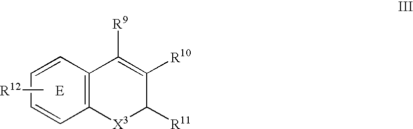

wherein X³ is selected from the group consisting of O or S or NR^{a};

wherein R^{a} is alkyl;

wherein R⁹ is selected from the group consisting of H and aryl;

wherein R¹⁰ is selected from the group consisting of carboxyl, aminocarbonyl, alkylsulfonylaminocarbonyl and alkoxycarbonyl;

wherein R¹¹ is selected from the group consisting of haloalkyl, alkyl, aralkyl, cycloalkyl and aryl optionally substituted with one or more radicals selected from alkylthio, nitro and alkylsulfonyl; and

wherein R¹² is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or

wherein R¹² together with ring E forms a naphthyl radical; or an isomer or pharmaceutically acceptable salt thereof; and including the diastereomers, enantiomers, racemates, tautomers, salts, esters, amides and prodrugs thereof.

A related class of compounds useful as Cox-2 selective inhibitors in the present invention is described by Formulas IV and V below:

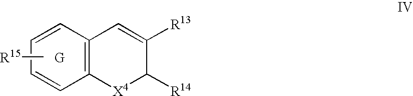

wherein X⁴ is selected from O or S or NR^{a};

wherein R^{a} is alkyl;

wherein R¹³ is selected from carboxyl, aminocarbonyl, alkylsulfonylaminocarbonyl and alkoxycarbonyl;

wherein R¹⁴ is selected from haloalkyl, alkyl, aralkyl, cycloalkyl and aryl optionally substituted with one or more radicals selected from alkylthio, nitro and alkylsulfonyl; and

wherein R¹⁵ is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;

or wherein R¹⁵ together with ring G forms a naphthyl radical; or an isomer or pharmaceutically acceptable salt thereof.

Formula V is:

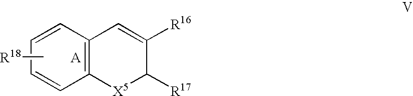

wherein:

X⁵ is selected from the group consisting of O or S or NR^{b};

R^{b} is alkyl;

R¹⁶ is selected from the group consisting of carboxyl, aminocarbonyl, alkylsulfonylaminocarbonyl and alkoxycarbonyl;

R¹⁷ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

R¹⁸ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R¹⁸ together with ring A forms a naphthyl radical;

or an isomer or pharmaceutically acceptable salt thereof.

The Cox-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of lower haloalkyl, lower cycloalkyl and phenyl; and

R¹⁸ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered nitrogen-containing heterocyclosulfonyl, lower alkylsulfonyl, optionally substituted phenyl, lower aralkylcarbonyl, and lower alkylcarbonyl; or

wherein R¹⁸ together with ring A forms a naphthyl radical; or an isomer or pharmaceutically acceptable salt thereof.

The Cox-2 selective inhibitor may also be a compound of Formula V, wherein:

X⁵ is selected from the group consisting of oxygen and sulfur;

R¹⁶ is carboxyl;

R¹⁷ is lower haloalkyl; and

R¹⁸ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R¹⁸ together with ring A forms a naphthyl radical;

or an isomer or pharmaceutically acceptable salt thereof.

The Cox-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and

R¹⁸ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or wherein R² together with ring A forms a naphthyl radical; or an isomer or pharmaceutically acceptable salt thereof.

The Cox-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting trifluoromethyl and pentafluoroethyl; and

R¹⁸ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R¹⁸ together with ring A forms a naphthyl radical;

or an isomer or prodrug thereof.

The Cox-2 selective inhibitor of the present invention can also be a compound having the structure of Formula VI:

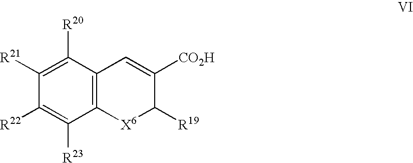

wherein:

X⁶ is selected from the group consisting of O and S;

R¹⁹ is lower haloalkyl;

R²⁰ is selected from the group consisting of hydrido, and halo;

R²¹ is selected from the group consisting of hydrido, halo, lower alkyl; lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;

R²² is selected from the group consisting of hydrido, lower alkyl, halo, lower alkoxy, and aryl; and

R²³ is selected from the group consisting of the group consisting of hydrido, halo, lower alkyl, lower alkoxy, and aryl;

or an isomer or prodrug thereof.

The Cox-2 selective inhibitor can also be a compound of having the structure of Formula VI, wherein:

X⁶ is selected from the group consisting of O and S;

R¹⁹ is selected from the group consisting of trifluoromethyl and pentafluoroethyl;

R²⁰ is selected from the group consisting of hydrido, chloro, and fluoro;

R²¹ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;

R²² is selected from the group consisting of hydrido, methyl, ethyl, isopropyl, tert-butyl, chloro, methoxy, diethylamino, and phenyl; and

R²³ is selected from the group consisting of hydrido, chloro, bromo, fluoro, methyl, ethyl, tert-butyl, methoxy, and phenyl;

or an isomer or prodrug thereof.

TABLE 1
Examples of Chromene Cox-2 Selective Inhibitors

| Compound | |
|---|---|
| Number | Structural Formula |
| B-3 | 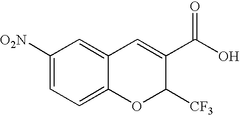 |
| | 6-Nitro-2-trifluoromethyl-2H-1-benzopyran- |
| | 3-carboxylic acid |
| B-4 | 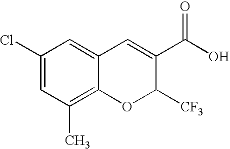 |
| | 6-Chloro-8-methyl-2-trifluoromethyl-2H-1- |
| | benzopyran-3-carboxylic acid |
| B-5 | 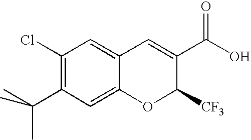 |
| | ((S)-6-Chloro-7-(1,1-dimethylethyl)-2- |
| | (trifluoromethyl-2H-1- |
| | benzopyran-3-carboxylic acid |
| B-6 | 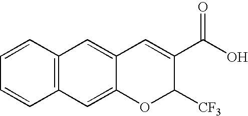 |
| | 2-Trifluoromethyl-2H-naphtho[2,3-b]pyran-3- |
| | carboxylic acid |
| B-7 | 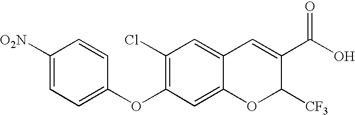 |
| | 6-Chloro-7-(4-nitrophenoxy)-2-(trifluoromethyl)- |
| | 2H-1-benzopyran-3- |
| | carboxylic acid |
| B-8 | 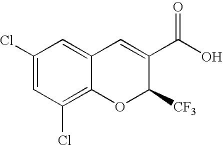 |
| | ((S)-6,8-Dichloro-2-(trifluoromethyl)-2H-1-benzopyran- |
| | 3-carboxylic acid |
| B-9 | 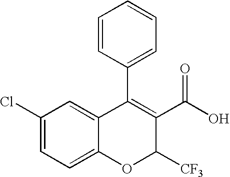 |
| | 6-Chloro-2-(trifluoromethyl)-4-phenyl-2H-1-benzopyran- |
| | 3-carboxylic acid |
| B-10 | 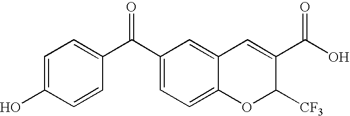 |
| | 6-(4-Hydroxybenzoyl)-2-(trifluoromethyl)-2H-1- |
| | benzopyran-3-carboxylic acid |
| B-11 | 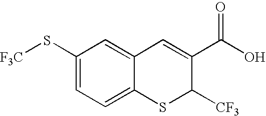 |
| | 2-(Trifluoromethyl)-6-[(trifluoro- |
| | methyl)thio]-2H-1-benzothiopyran- |
| | 3-carboxylic acid |
| B-12 | 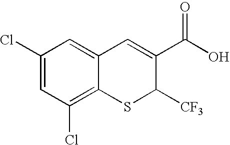 |
| | 6,8-Dichloro-2-trifluoromethyl-2H-1-benzothiopyran- |
| | 3-carboxylic acid |
| B-13 | 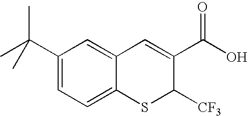 |
| | 6-(1,1-Dimethylethyl)-2-(trifluoromethyl)-2H-1- |
| | benzothiopyran-3-carboxylic acid |
| B-14 | 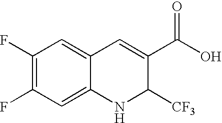 |
| | 6,7-Difluoro-1,2-dihydro-2-(trifluoromethyl)-3- |
| | quinolinecarboxylic acid |
| B-15 | 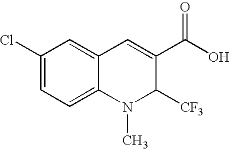 |
| | 6-Chloro-1,2-dihydro-1-methyl-2-(trifluoromethyl)-3- |
| | quinolinecarboxylic acid |
| B-16 | 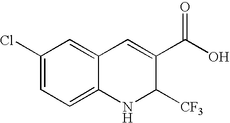 |
| | 6-Chloro-2-(trifluoromethyl)- |
| | 1,2-dihydro[1,8]naphthyridine- |
| | 3-carboxylic acid |
| B-17 | 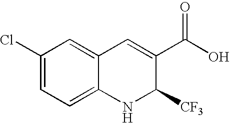 |
| | ((S)-6-Chloro-1,2-dihydro-2-(trifluoromethyl)-3- |
| | quinolinecarboxylic acid |
| B-18 | 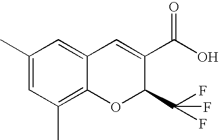 |
| | (2S)-6,8-dimethyl-2-(trifluoromethyl)-2H-chromene-3- |
| | carboxylic acid |
| B-19 | 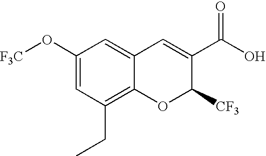 |
| | (2S)-8-ethyl-6-(trifluoromethoxy)-2-(trifluoromethyl)-2H- |
| | chromene-3-carboxylic acid |
| B-20 | 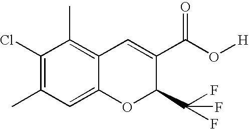 |
| | (2S)-6-chloro-5,7-dimethyl-2- |
| | (trifluoromethyl)-2H-chromene-3- |
| | carboxylic acid |

In preferred embodiments the chromene Cox-2 inhibitor is selected from (S)-6-chloro-7-(1,1-dimethylethyl)-2-(trifluoromethyl)-2H-1-benzopyran-3-carboxylic acid, (2S)-6,8-dimethyl-2-(trifluoromethyl)-2H-chromene-3-carboxylic acid, (2S)-6-chloro-8-methyl-2-(trifluoromethyl)-2H-chromene-3-carboxylic acid, (2S)-8-ethyl-6-(trifluoromethoxy)-2-(trifluoromethyl)-2H-chromene-3-carboxylic acid, (S)-6,8-dichloro-2-(trifluoromethyl)-2H-1-benzopyran-3-carboxylic acid, (2S)-6-chloro-5,7-dimethyl-2-(trifluoromethyl)-2H-chromene-3-carboxylic acid, and mixtures thereof.

In a preferred embodiment of the invention the Cox-2 inhibitor can be selected from the class of tricyclic Cox-2 selective inhibitors represented by the general structure of formula VII:

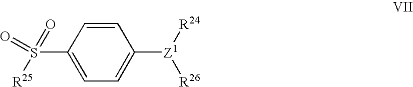

wherein:

Z¹ is selected from the group consisting of partially unsaturated or unsaturated heterocyclyl and partially unsaturated or unsaturated carbocyclic rings;

R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R²⁵ is selected from the group consisting of methyl or amino; and

R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;

or a prodrug thereof.

In a preferred embodiment of the invention the Cox-2 selective inhibitor represented by the above Formula VII is selected from the group of compounds, illustrated in Table 2, which includes celecoxib (B-21), valdecoxib (B-22), deracoxib (B-23), rofecoxib (B-24), etoricoxib (MK-663; B-25), JTE-522 (B-26), or prodrugs thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 2
Examples of Tricyclic COX-2 Selective Inhibitors

| Compound | |
|---|---|
| Number | Structural Formula |
| B-21 | 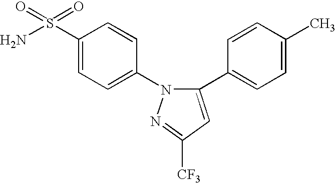 |
| B-22 | 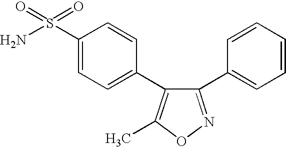 |
| B-23 | 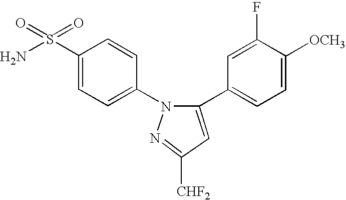 |
| B-24 | 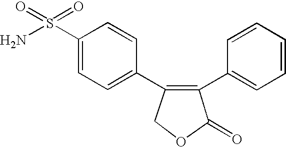 |
| B-25 | 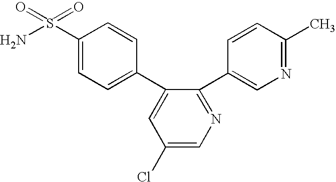 |
| B-26 | 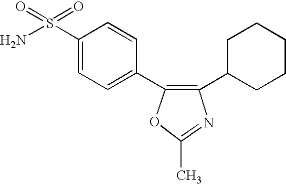 |

In a more preferred embodiment of the invention, the Cox-2 selective inhibitor is selected from the group consisting of celecoxib, rofecoxib and etoricoxib.

In a preferred embodiment, parecoxib (See, U.S. Pat. No. 5,932,598), having the structure shown in B-27, and which is a therapeutically effective prodrug of the tricyclic Cox-2 selective inhibitor valdecoxib, B-22, (See, U.S. Pat. No. 5,633,272), may be advantageously employed as the Cox-2 inhibitor of the present invention.

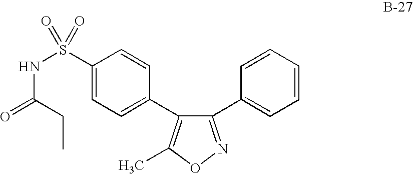

A preferred form of parecoxib is sodium parecoxib.

Another tricyclic Cox-2 selective inhibitor useful in the present invention is the compound ABT-963, having the formula B-28 shown below, that has been previously described in International Publication Number WO 00/24719.

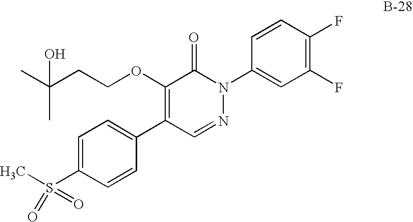

In a further embodiment of the invention, the Cox-2 inhibitor can be selected from the class of phenylacetic acid derivative Cox-2 selective inhibitors represented by the general structure of formula VIII:

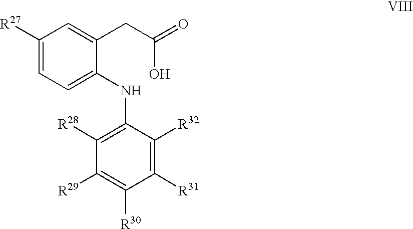

wherein:

R²⁷ is methyl, ethyl, or propyl;

R²⁸ is chloro or fluoro;

R²⁹ is hydrogen, fluoro, or methyl;

R³⁰ is hydrogen, fluoro, chloro, methyl, ethyl, methoxy, ethoxy or hydroxyl;

R³¹ is hydrogen, fluoro, or methyl; and

R³² is chloro, fluoro, trifluoromethyl, methyl, or ethyl,

provided that R²⁸, R²⁹, R³⁰ and R³¹ are not all fluoro when R²⁷ is ethyl and R³⁰ is H.

An exemplary phenylacetic acid derivative Cox-2 selective inhibitor that is described in WO 99/11605 is a compound that has the structure shown in formula VIII,

wherein:

R²⁷ is ethyl;

R²⁸ and R³⁰ are chloro;

R²⁹ and R³¹ are hydrogen; and

R³² is methyl.

Another phenylacetic acid derivative Cox-2 selective inhibitor is a compound that has the structure shown in formula VIII,

wherein:

R²⁷ is propyl;

R²⁸ and R³⁰ are chloro;

R²⁹ and R³¹ are methyl; and

R³² is ethyl.

Another phenylacetic acid derivative Cox-2 selective inhibitor that is disclosed in WO 02/20090 is a compound that is referred to as COX-189 (also termed lumiracoxib; CAS Reg. No. 220991-20-8), having the structure shown in formula VIII,

wherein:

R²⁷ is methyl;

R²⁸ is fluoro;

R³² is chloro; and

R²⁹, R³⁰, and R³¹ are hydrogen.

Compounds having a structure similar to that shown in formula VIII, that can serve as the Cox-2 selective inhibitor of the present invention, are described in U.S. Pat. Nos. 6,451,858, 6,310,099, 6,291,523, and 5,958,978.

Other Cox-2 selective inhibitors that can be used in the present invention have the general structure shown in formula IX, where the J group is a carbocycle or a heterocycle. Preferred embodiments have the structure:

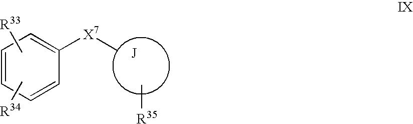

wherein:

X⁷ is 0; J is 1-phenyl; R³³ is 2-NHSO₂CH₃; R³⁴ is 4-NO₂; and there is no R³⁵ group, (nimesulide), or

X⁷ is 0; J is 1-oxo-inden-5-yl; R³³ is 2-F; R³⁴ is 4-F; and R³⁵ is 6-NHSO₂CH₃, (flosulide); or

X⁷ is O; J is cyclohexyl; R³³ is 2-NHSO₂CH₃; R³⁴ is 5-NO₂; and there is no R³⁵ group, (NS-398); or

X⁷ is S; J is 1-oxo-inden-5-yl; R³³ is 2-F; R³⁴ is 4-F; and R³⁵ is 6-N⁻SO₂CH₃.Na⁺, (L-745337); or

X⁷ is S; J is thiophen-2-yl; R³³ is 4-F; there is no R³⁴ group; and R³⁵ is 5-NHSO₂CH₃, (RWJ-63556); or

X⁷ is 0; J is 2-oxo-5(R)-methyl-5-(2,2,2-trifluoroethyl)furan-(5H)-3-yl; R³³ is 3-F; R³⁴ is 4-F; and R³⁵ is 4-(p-SO₂CH₃)C₆H₄, (L-784512).

The Cox-2 selective inhibitor NS-398, also known as N-(2-cyclohexyloxynitrophenyl)methane sulfonamide (CAS RN 123653-11-2), having a structure as shown below in formula B-29, has been described in, for example, Yoshimi, N. et al., in Japanese J. Cancer Res., 90(4):406-412 (1999).

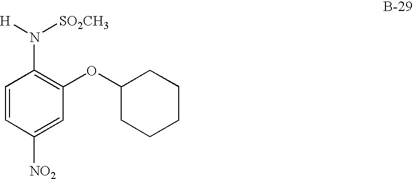

An evaluation of the anti-inflammatory activity of the Cox-2 selective inhibitor, RWJ 63556, in a canine model of inflammation, was described by Kirchner et al., in J Pharmacol Exp Ther 282, 1094-1101 (1997).

Materials that can serve as the Cox-2 selective inhibitor of the present invention include diarylmethylidenefuran derivatives that are described in U.S. Pat. No. 6,180,651. Such diarylmethylidenefuran derivatives have the general formula shown below in formula X:

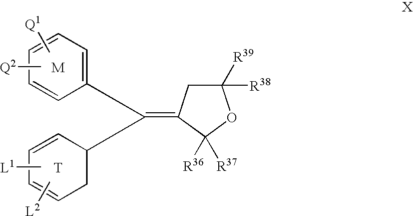

wherein:

the rings T and M independently are a phenyl radical, a naphthyl radical, a radical derived from a heterocycle comprising 5 to 6 members and possessing from 1 to 4 heteroatoms, or a radical derived from a saturated hydrocarbon ring having from 3 to 7 carbon atoms;

at least one of the substituents Q¹, Q², L¹ or L² is an —S(O)ₙ—R group, in which n is an integer equal to 0, 1 or 2 and R is a lower alkyl radical having 1 to 6 carbon atoms, a lower haloalkyl radical having 1 to 6 carbon atoms, or an —SO₂NH₂ group;

and is located in the para position,

the others independently being a hydrogen atom, a halogen atom, a lower alkyl radical having 1 to 6 carbon atoms, a trifluoromethyl radical, or a lower O-alkyl radical having 1 to 6 carbon atoms, or Q¹ and Q² or L¹ and L² are a methylenedioxy group; and

R³⁶, R³⁷, R³⁸ and R³⁹ independently are a hydrogen atom, a halogen atom, a lower alkyl radical having 1 to 6 carbon atoms, a lower haloalkyl radical having 1 to 6 carbon atoms, or an aromatic radical selected from the group consisting of phenyl, naphthyl, thienyl, furyl and pyridyl; or,

R³⁶, R³⁷ or R³⁸, R³⁹ are an oxygen atom; or

R³⁶, R³⁷ or R³⁸, R³⁹, together with the carbon atom to which they are attached, form a saturated hydrocarbon ring having from 3 to 7 carbon atoms;

or an isomer or prodrug thereof.

Particular diarylmethylidenefuran derivatives that can serve as the Cox-2 selective inhibitor of the present invention include, for example, N-(2-cyclohexyloxynitrophenyl)methane sulfonamide, and (E)-4-[(4-methylphenyl)(tetrahydro-2-oxo-3-furanylidene) methyl]benzenesulfonamide.

Other Cox-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Compounds that may act as Cox-2 selective inhibitors of the present invention include multibinding compounds containing from 2 to 10 ligands covanlently attached to one or more linkers, as described in U.S. Pat. No. 6,395,724.

Conjugated linoleic, as described in U.S. Pat. No. 6,077,868, is useful as a Cox-2 selective inhibitor in the present invention.

Compounds that can serve as a Cox-2 selective inhibitor of the present invention include heterocyclic aromatic oxazole compounds that are described in U.S. Pat. Nos. 5,994,381 and 6,362,209. Such heterocyclic aromatic oxazole compounds have the formula shown below in formula XI:

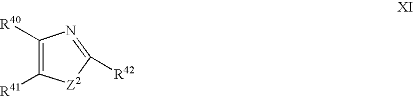

wherein:

Z² is an oxygen atom;

one of R⁴⁰ and R⁴¹ is a group of the formula

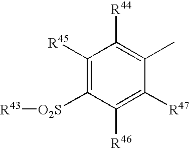

wherein:

R⁴³ is lower alkyl, amino or lower alkylamino; and

R⁴⁴, R⁴⁵, R⁴⁶ and R⁴⁷ are the same or different and each is hydrogen atom, halogen atom, lower alkyl, lower alkoxy, trifluoromethyl, hydroxyl or amino, provided that at least one of R⁴⁴, R⁴⁵, R⁴⁶ and R⁴⁷ is not hydrogen atom, and the other is an optionally substituted cycloalkyl, an optionally substituted heterocyclic group or an optionally substituted aryl; and

R³⁰ is a lower alkyl or a halogenated lower alkyl,

and a pharmaceutically acceptable salt thereof.

Cox-2 selective inhibitors that are useful in the method and compositions of the present invention include compounds that are described in U.S. Pat. Nos. 6,080,876 and 6,133,292, and described by formula XII:

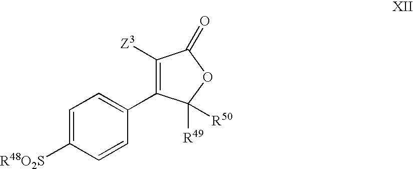

wherein:

Z³ is selected from the group consisting of linear or branched C₁-C₆ alkyl, linear or branched C₁-C₆ alkoxy, unsubstituted, mono-, di- or tri-substituted phenyl or naphthyl wherein the substituents are selected from the group consisting of hydrogen, halo, C₁-C₃ alkoxy, CN, C₁-C₃ fluoroalkyl C₁-C₃ alkyl, and —CO₂H;

R⁴⁸ is selected from the group consisting of NH₂ and CH₃,

R⁴⁹ is selected from the group consisting of C₁-C₆ alkyl unsubstituted or substituted with C₃-C₆ cycloalkyl, and C₃-C₆ cycloalkyl;

R⁵⁰ is selected from the group consisting of: C₁-C₆ alkyl unsubstituted or substituted with one, two or three fluoro atoms, and C₃-C₆ cycloalkyl;

with the proviso that R⁴⁹ and R⁵⁰ are not the same.

Pyridines that are described in U.S. Pat. Nos. 6,596,736, 6,369,275, 6,127,545, 6,130,334, 6,204,387, 6,071,936, 6,001,843 and 6,040,450, and can seve as Cox-2 selective inhibitors of the present invention, have the general formula described by formula XIII:

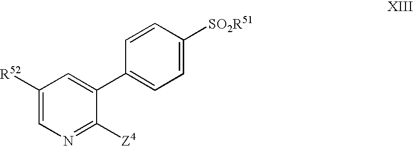

wherein:

R⁵¹ is selected from the group consisting of CH₃, NH₂, NHC(O)CF₃, and NHCH₃;

Z⁴ is a mono-, di-, or trisubstituted phenyl or pyridinyl (or the N-oxide thereof), wherein the substituents are chosen from the group consisting of hydrogen, halo, C₁-C₆ alkoxy, C₁-C₆ alkylthio, CN, C₁-C₆ alkyl, C₁-C₆ fluoroalkyl, N₃, —CO₂R⁵³, hydroxyl, —C(R⁵⁴)(R⁵⁵)—OH, —C₁-C₆ alkyl-CO₂—R⁵⁶, C₁-C₆ fluoroalkoxy;

R⁵² is chosen from the group consisting of: halo, C₁-C₆ alkoxy, C₁-C₆ alkylthio, CN, C₁-C₆ alkyl, C₁-C₆ fluoroalkyl, N₃, —CO₂R⁵⁷, hydroxyl, —C(R⁵⁸)(R⁵⁹)—OH, —C₁-C₆ alkyl-CO₂—R⁶⁰, C₁-C₆ fluoroalkoxy, NO₂, NR⁶¹R⁶², and NHCOR⁶³;

R⁵³, R⁵⁴, R⁵⁵, R⁵⁶, R⁵⁷, R⁵⁸, R⁵⁹, R⁶⁰, R⁶¹, R⁶², and R⁶³, are each independently chosen from the group consisting of hydrogen and C₁-C₆ alkyl;

or R⁵⁴ and R⁵⁵, R⁵⁸ and R⁵⁹, or R⁶¹ and R⁶² together with the atom to which they are attached form a saturated monocyclic ring of 3, 4, 5, 6, or 7 atoms.

Materials that can serve as the Cox-2 selective inhibitor of the present invention include diarylbenzopyran derivatives that are described in U.S. Pat. No. 6,340,694. Such diarylbenzopyran derivatives have the general formula shown below in formula XIV:

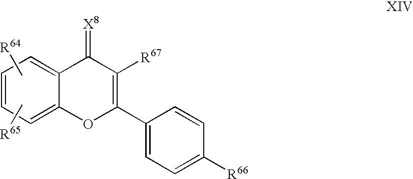

wherein:

X⁸ is an oxygen atom or a sulfur atom;

R⁶⁴ and R⁶⁵, identical to or different from each other, are independently a hydrogen atom, a halogen atom, a C₁-C₆ lower alkyl group, a trifluoromethyl group, an alkoxy group, a hydroxyl group, a nitro group, a nitrile group, or a carboxyl group;

R⁶⁶ is a group of a formula: S(O)ₙR⁶⁸ wherein n is an integer of 0˜2, R⁶⁸ is a hydrogen atom, a C₁-C₆ lower alkyl group, or a group of a formula: NR⁶⁹R⁷⁰ wherein R⁶⁹ and R⁷⁰, identical to or different from each other, are independently a hydrogen atom, or a C₁-C₆ lower alkyl group; and

R⁶⁷ is oxazolyl, benzo[b]thienyl, furanyl, thienyl, naphthyl, thiazolyl, indolyl, pyrolyl, benzofuranyl, pyrazolyl, pyrazolyl substituted with a C₁-C₆ lower alkyl group, indanyl, pyrazinyl, or a substituted group represented by the following structures:

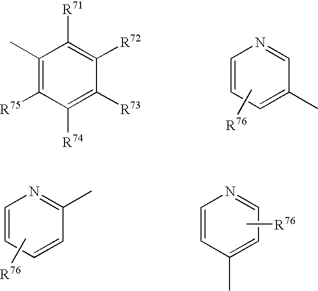

wherein:

R⁷¹ through R⁷⁵, identical to or different from one another, are independently a hydrogen atom, a halogen atom, a C₁-C₆ lower alkyl group, a trifluoromethyl group, an alkoxy group, a hydroxyl group, a hydroxyalkyl group, a nitro group, a group of a formula: S(O)ₙR⁶⁸, a group of a formula: NR⁶⁹R⁷⁰, a trifluoromethoxy group, a nitrile group a carboxyl group, an acetyl group, or a formyl group,

wherein n, R⁶⁸, R⁶⁹ and R⁷⁰ have the same meaning as defined by R⁶⁶ above; and

R⁷⁶ is a hydrogen atom, a halogen atom, a C₁-C₆ lower alkyl group, a trifluoromethyl group, an alkoxy group, a hydroxyl group, a trifluoromethoxy group, a carboxyl group, or an acetyl group.

Materials that can serve as the Cox-2 selective inhibitor of the present invention include 1-(4-sulfamylaryl)-3-substituted-5-aryl-2-pyrazolines that are described in U.S. Pat. No. 6,376,519. Such 1-(4-sulfamylaryl)-3-substituted-5-aryl-2-pyrazolines have the formula shown below in formula XV:

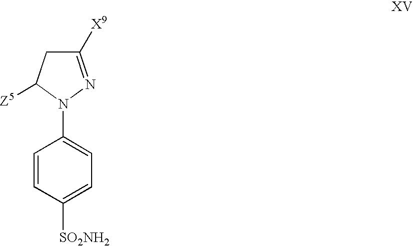

wherein:

X⁹ is selected from the group consisting of C₁-C₆ trihalomethyl, preferably trifluoromethyl; C₁-C₆ alkyl; and an optionally substituted or di-substituted phenyl group of formula XVI:

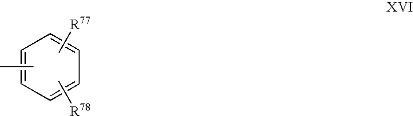

wherein:

R⁷⁷ and R⁷⁸ are independently selected from the group consisting of hydrogen, halogen, preferably chlorine, fluorine and bromine; hydroxyl; nitro; C₁-C₆ alkyl, preferably C₁-C₃ alkyl; C₁-C₆ alkoxy, preferably C₁-C₃ alkoxy; carboxy; C₁-C₆ trihaloalkyl, preferably trihalomethyl, most preferably trifluoromethyl; and cyano;

Z⁵ is selected from the group consisting of substituted and unsubstituted aryl.

Compounds useful as Cox-2 selective inhibitors of the present invention include heterocycles that are described in U.S. Pat. No. 6,153,787. Such heterocycles have the general formulas shown below in formulas XVII and XVIII:

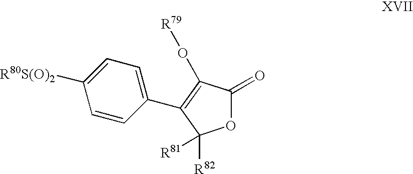

wherein:

R⁷⁹ is a mono-, di-, or tri-substituted C₁-C₁₂ alkyl, or a mono-, or an unsubstituted or mono-, di- or tri-substituted linear or branched C₂-C₁₀ alkenyl, or an unsubstituted or mono-, di- or tri-substituted linear or branched C₂-C₁₀ alkynyl, or an unsubstituted or mono-, di- or tri-substituted C₃-C₁₂ cycloalkenyl, or an unsubstituted or mono-, di- or tri-substituted C₅-C₁₂ cycloalkynyl, wherein the substituents are chosen from the group consisting of halo selected from F, Cl, Br, and 1, OH, CF₃, C₃-C₆ cycloalkyl, ═O, dioxolane, CN;

R⁸⁰ is selected from the group consisting of CH₃, NH₂, NHC(O)CF₃, and NHCH₃;

R⁸¹ and R⁸² are independently chosen from the group consisting of hydrogen and C₁-C₁₀ alkyl;

or R⁸¹ and R⁸² together with the carbon to which they are attached form a saturated monocyclic carbon ring of 3, 4, 5, 6 or 7 atoms.

Formula XVIII is:

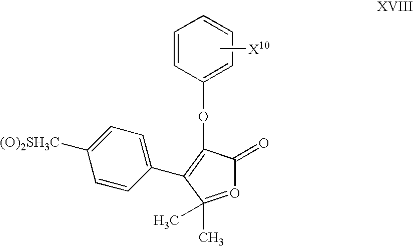

wherein X¹⁰ is fluoro or chloro.

Materials that can serve as the Cox-2 selective inhibitor of the present invention include 2,3,5-trisubstituted pyridines that are described in U.S. Pat. No. 6,046,217. Such pyridines have the general formula shown below in formula XIX:

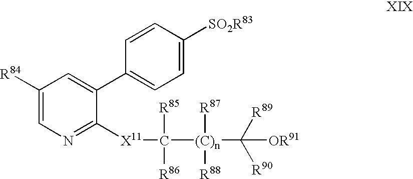

or a pharmaceutically acceptable salt thereof,

wherein:

X¹¹ is selected from the group consisting of O, S, and a bond;

n is 0 or 1;

R⁸³ is selected from the group consisting of CH₃, NH₂, and NHC(O)CF₃;

R⁸⁴ is chosen from the group consisting of halo, C₁-C₆ alkoxy, C₁-C₆ alkylthio, CN, C₁-C₆ alkyl, C₁-C₆ fluoroalkyl, N₃, —CO₂R⁹², hydroxyl, —C(R⁹³)(R⁹⁴)—OH, —C₁-C₆ alkyl-CO₂—R⁹⁵, C₁-C₆ fluoroalkoxy, NO₂, NR⁹⁶R⁹⁷, and NHCOR⁹⁸;

R⁸⁵ to R⁸⁹ are independently chosen from the group consisting of hydrogen and C₁-C₆ alkyl;

or R⁸⁵ and R⁸⁹, or R⁸⁹ and R⁹⁰ together with the atoms to which they are attached form a carbocyclic ring of 3, 4, 5, 6 or 7 atoms, or R⁸⁵ and R⁸⁷ are joined to form a bond.

Compounds that are useful as the Cox-2 selective inhibitor of the present invention include diaryl bicyclic heterocycles that are described in U.S. Pat. No. 6,329,421. Such diaryl bicyclic heterocycles have the general formula shown below in formula XX:

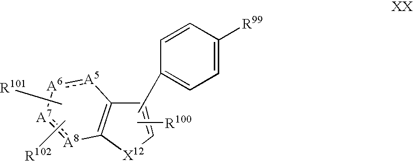

and pharmaceutically acceptable salts thereof wherein:

-A⁵=A⁶-A⁷=A⁸- is selected from the group consisting of:

(a) —CH═CH—CH═CH—,

(b) —CH₂—CH₂—CH₂—C(O)—, —CH₂—CH₂—C(O)—CH₂—, —CH₂—C(O)—CH₂—CH₂, —C(O)—CH₂—CH₂—CH₂,

(c) —CH₂—CH₂—C(O)—, —CH₂—C(O)—CH₂—, —C(O)—CH₂—CH₂—

(d) —CH₂—CH₂—O—C(O)—, CH₂—O—C(O)—CH₂—, —O—C(O)—CH₂—CH₂—,

(e) —CH₂—CH₂—C(O)—O—, —CH₂—C(O)—OCH₂—, —C(O)—O—CH₂—CH₂—,

(f) —C(R¹⁰⁵)₂—O—C(O)—, —C(O)—O—C(R¹⁰⁵)₂, —O—C(O)—C(R¹⁰⁵)₂—, —C(R¹⁰⁵)₂—C(O)—O—,

(g) —N═CH—CH═CH—,

(h) —CH═N—CH═CH—,

(i) —CH═CH—N═CH—,

(j) —CH═CH—CH═N—,

(k) —N═CH—CH═N—,

(l) —N═CH—N═CH—,

(m) —CH═N—CH═N—,

(n) —S—CH═N—,

(o) —S—N═CH—,

(p) —N═N—NH—,

(q) —CH═N—S—, and

(r) —N═CH—S—;

R⁹⁹ is selected from the group consisting of S(O)₂CH₃, S(O)₂NH₂, S(O)₂NHCOCF₃, S(O)(NH)CH₃, S(O)(NH)NH₂, S(O)(NH)NHCOCF₃, P(O)(CH₃)OH, and P(O)(CH₃)NH₂;

R¹⁰⁰ is selected from the group consisting of:

(a) C₁-C₆ alkyl,

(b) C₃-C₇ cycloalkyl,

(c) mono- or di-substituted phenyl or naphthyl wherein the substituent is selected from the group consisting of:

(1) hydrogen,

(2) halo, including F, Cl, Br, I,

(3) C₁-C₆ alkoxy,

(4) C₁-C₆ alkylthio,

(5) CN,

(6) CF₃,

(7) C₁-C₆ alkyl,

(8) N₃,

(9) —CO₂H,

(10) —CO₂—C₁-C₄ alkyl,

(11) —C(R¹⁰³)(R¹⁰⁴)—OH,

(12) —C(R¹⁰³)(R¹⁰⁴)—O—C₁-C₄ alkyl, and

(13) —C₁-C₆ alkyl-CO₂—R¹⁰⁶;

(d) mono- or di-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁-C₆ alkyl,

(4) C₁-C₆ alkoxy,

(5) C₁-C₆ alkylthio,

(6) CN,

(7) CF₃,

(8) N₃,

(9) —C(R¹⁰³)(R¹⁰⁴)—OH, and

(10) —C(R¹⁰³)(R¹⁰⁴)—O—C₁-C₄ alkyl;

(e) benzoheteroaryl which includes the benzo fused analogs of (d);

R¹⁰¹ and R¹⁰² are the substituents residing on any position of -A⁵=A⁶-A⁷=A- and are selected independently from the group consisting of:

(a) hydrogen,

(b) CF₃,

(c) CN,

(d) C₁-C₆ alkyl,

(e) -Q³ wherein Q³ is Q⁴, CO₂H, C(R¹⁰³)(R¹⁰⁴)OH,

(f) —O-Q⁴,

(g) —S-Q⁴, and

(h) optionally substituted:

(1) —C₁-C₅ alkyl-Q³,

(2) —O—C₁-C₅ alkyl-Q³,

(3) —S—C₁-C₅ alkyl-Q³,

(4) —C₁-C₃ alkyl-O—C₁₋₃ alkyl-Q³,

(5) —C₁-C₃ alkyl-S—C₁₋₃ alkyl-Q³,

(6) —C₁-C₅ alkyl-O-Q⁴,

(7) —C₁-C₅ alkyl-S-Q⁴,

wherein the substituent resides on the alkyl chain and the substituent is C₁-C₃ alkyl, and Q³ is Q⁴, CO₂H, C(R¹⁰³)(R¹⁰⁴)OH Q⁴ is CO₂—C₁-C₄ alkyl, tetrazolyl-5-yl, or C(R¹⁰³)(R¹⁰⁴)O—C₁-C₄ alkyl;

R¹⁰³, R¹⁰⁴ and R¹⁰⁵ are each independently selected from the group consisting of hydrogen and C₁-C₆ alkyl; or

R¹⁰³ and R¹⁰⁴ together with the carbon to which they are attached form a saturated monocyclic carbon ring of 3, 4, 5, 6 or 7 atoms, or two R¹⁰⁵ groups on the same carbon form a saturated monocyclic carbon ring of 3, 4, 5, 6 or 7 atoms;

R¹⁰⁶ is hydrogen or C₁-C₆ alkyl;

R¹⁰⁷ is hydrogen, C₁-C₆ alkyl or aryl;

X⁷ is O, S, NR¹⁰⁷, CO, C(R¹⁰⁷)₂, C(R¹⁰⁷)(OH), —C(R¹⁰⁷)═C(R¹⁰⁷)—; —C(R¹⁰⁷)═N—; or —N═C(R¹⁰⁷)—.

Compounds that may act as Cox-2 selective inhibitors include salts of 5-amino or a substituted amino 1,2,3-triazole compound that are described in U.S. Pat. No. 6,239,137. The salts are of a class of compounds of formula XXI:

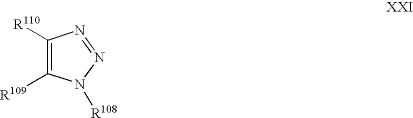

wherein:

R¹⁰⁸ is:

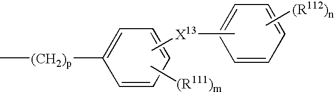

wherein:

p is 0 to 2; m is 0 to 4; and n is 0 to 5;

X¹³ is O, S, SO, SO₂, CO, CHCN, CH₂ or C═NR¹¹³ where R¹¹³ is hydrogen, loweralkyl, hydroxyl, loweralkoxy, amino, loweralkylamino, diloweralkylamino or cyano;

R¹¹¹ and R¹¹² are independently halogen, cyano, trifluoromethyl, loweralkanoyl, nitro, loweralkyl, loweralkoxy, carboxy, lowercarbalkoxy, trifuloromethoxy, acetamido, loweralkylthio, loweralkylsulfinyl, loweralkylsulfonyl, trichlorovinyl, trifluoromethylthio, trifluoromethylsulfinyl, or trifluoromethylsulfonyl;

R¹⁰⁹ is amino, mono or diloweralkyl amino, acetamido, acetimido, ureido, formamido, or guanidino; and

R¹¹⁰ is carbamoyl, cyano, carbazoyl, amidino or N-hydroxycarbamoyl;

wherein the loweralkyl, loweralkyl containing, loweralkoxy and loweralkanoyl groups contain from 1 to 3 carbon atoms.

Pyrazole derivatives such as those described in U.S. Pat. No. 6,136,831 can serve as a Cox-2 selective inhibitor of the present invention. Such pyrazole derivatives have the formula shown below in formula XXII:

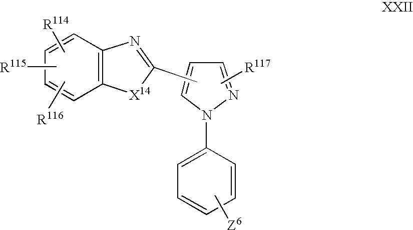

wherein:

R¹¹⁴ is hydrogen or halogen;

R¹¹⁵ and R¹¹⁶ are each independently hydrogen, halogen, lower alkyl, lower alkoxy, hydroxyl or lower alkanoyloxy;

R¹¹⁷ is lower haloalkyl or lower alkyl;

X¹⁴ is sulfur, oxygen or NH; and

Z⁶ is lower alkylthio, lower alkylsulfonyl or sulfamoyl;

or a pharmaceutically acceptable salt thereof.

Materials that can serve as a Cox-2 selective inhibitor of the present invention include substituted derivatives of benzosulphonamides that are described in U.S. Pat. No. 6,297,282. Such benzosulphonamide derivatives have the formula shown below in formula XXIII:

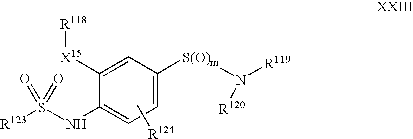

wherein:

X¹⁵ denotes oxygen, sulphur or NH;

R¹¹⁸ is an optionally unsaturated alkyl or alkyloxyalkyl group, optionally mono- or polysubstituted or mixed substituted by halogen, alkoxy, oxo or cyano, a cycloalkyl, aryl or heteroaryl group optionally mono- or polysubstituted or mixed substituted by halogen, alkyl, CF₃, cyano or alkoxy;

R¹¹⁹ and R¹²⁰, independently from one another, denote hydrogen, an optionally polyfluorised alkyl group, an aralkyl, aryl or heteroaryl group or a group (CH₂)ₙ—X¹⁶; or

R¹¹⁹ and R¹²⁰, together with the N— atom, denote a 3 to 7-membered, saturated, partially or completely unsaturated heterocycle with one or more heteroatoms N, O or S, which can optionally be substituted by oxo, an alkyl, alkylaryl or aryl group, or a group (CH₂)ₙ—X¹⁶;

X¹⁶ denotes halogen, NO₂, —OR¹²¹, —COR¹²¹, —CO₂R¹²¹, —OCO₂R¹²¹, —CN, —CONR¹²¹R¹²², —CONR¹²¹R¹²², —SR¹²¹, —S(O)R¹²¹, —S(O)₂R¹²¹, —NR¹²¹R¹²², —NHC(O)R¹²¹, —NHS(O)₂R¹²¹;

n denotes a whole number from 0 to 6;

R¹²³ denotes a straight-chained or branched alkyl group with 1-10 C-atoms, a cycloalkyl group, an alkylcarboxyl group, an aryl group, aralkyl group, a heteroaryl or heteroaralkyl group which can optionally be mono- or polysubstituted or mixed substituted by halogen or alkoxy;

R¹²⁴ denotes halogen, hydroxyl, a straight-chained or branched alkyl, alkoxy, acyloxy or alkyloxycarbonyl group with 1-6 C-atoms, which can optionally be mono- or polysubstituted by halogen, NO₂, —OR¹²¹, —COR¹²¹, —CO₂R¹²¹, —OCO₂R¹²¹, —CN, —CONR¹²¹R¹²², —CONR¹²¹R¹²², —SR¹²¹, —S(O)R¹²¹, —S(O)₂R¹²¹, —NR¹²¹R¹²², —NHC(O)R¹²¹, —NHS(O)₂R¹²¹, or a polyfluoroalkyl group;

R¹²¹ and R¹²², independently from one another, denote hydrogen, alkyl, aralkyl or aryl; and

m denotes a whole number from 0 to 2;

and the pharmaceutically-acceptable salts thereof.

Compounds that are useful as Cox-2 selective inhibitors of the present invention include phenyl heterocycles that are described in U.S. Pat. Nos. 5,474,995 and 6,239,173. Such phenyl heterocyclic compounds have the formula shown below in formula XXIV:

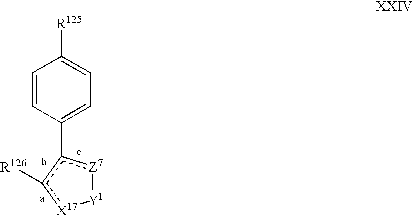

or pharmaceutically acceptable salts thereof wherein:

X¹⁷—Y¹-Z⁷-is selected from the group consisting of:

(a) —CH₂ CH₂ CH₂—,

(b) —C(O)CH₂ CH₂—,

(c) —CH₂CH₂C(O)—,

(d) —CR¹²⁹(R^{129′})—O—C(O)—,

(e) —C(O)—O—CR¹²⁹(R^{129′})—,

(f) —CH₂—NR¹²⁷—CH₂—,

(g) —CR¹²⁹(R^{129′})—NR¹²⁷—C(O)—,

(h) —CR¹²⁸═CR^{128′}—S—,

(i) —S—CR¹²⁸═CR^{128′}—,

(j) —S—N═CH—,

(k) —CH═N—S—,

(l) —N═CR¹²⁸—O—,

(m) —O—CR¹²⁸═N—,

(n) —N═CR¹²⁸—NH—,

(o) —N═CR¹²⁸—S—, and

(p) —S—CR¹²⁸═N—,

(q) —C(O)—NR¹²⁷—CR¹²⁹(R^{129′})—,

(r) —R¹²⁷N—CH═CH— provided R¹²² is not —S(O)₂CH₃,

(s) —CH═CH—NR¹²⁷— provided R¹²⁵ is not —S(O)₂CH₃;

when side b is a double bond, and sides a and c are single bonds; and

X¹⁷—Y¹-Z⁷-is selected from the group consisting of:

(a) ═CH—O—CH═, and

(b) ═CH—NR¹²⁷—CH═,

(c) ═N—S—CH═,

(d) ═CH—S—N═,

(e) ═N—O—CH═,

(f) ═CH—O—N═,

(g) ═N—S—N═,

(h) ═N—O—N═,

when sides a and c are double bonds and side b is a single bond;

R¹²⁵ is selected from the group consisting of:

(a) S(O)₂ CH₃,

(b) S(O)₂ NH₂,

(c) S(O)₂ NHC(O)CF₃,

(d) S(O)(NH)CH₃,

(e) S(O)(NH)NH₂,

(f) S(O)(NH)NHC(O)CF₃,

(g) P(O)(CH₃)OH, and

(h) P(O)(CH₃)NH₂;

R¹²⁶ is selected from the group consisting of

(a) C₁-C₆ alkyl,

(b) C₃, C₄, C₅, C₆, and C₇, cycloalkyl,

(c) mono-, di- or tri-substituted phenyl or naphthyl, wherein the substituent is selected from the group consisting of:

(1) hydrogen,

(2) halo,

(3) C₁-C₆ alkoxy,

(4) C₁-C₆ alkylthio,

(5) CN,

(6) CF₃,

(7) C₁-C₆ alkyl,

(8) N₃,

(9) —CO₂H,

(10) —CO₂—C₁-C₄ alkyl,

(11) —C(R¹²⁹)(R¹³⁰)—OH,

(12) —C(R¹²⁹)(R¹³⁰)—O—C₁-C₄ alkyl, and

(13) —C₁-C₆ alkyl-CO₂—R¹²⁹;

(d) mono-, di- or tri-substituted heteroaryl wherein the heteroaryl is a monocyclic aromatic ring of 5 atoms, said ring having one hetero atom which is S, O, or N, and optionally 1, 2, or 3 additionally N atoms; or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁-C₆ alkyl,

(4) C₁-C₆ alkoxy,

(5) C₁-C₆ alkylthio,

(6) CN,

(7) CF₃,

(8) N₃,

(9) —C(R¹²⁹)(R¹³⁰)—OH, and

(10) —C(R¹²⁹)(R¹³⁰)—O—C₁-C₄ alkyl;

(e) benzoheteroaryl which includes the benzo fused analogs of (d);

R¹²⁷ is selected from the group consisting of:

(a) hydrogen,

(b) CF₃,

(c) CN,

(d) C₁-C₆ alkyl,

(e) hydroxyl C₁-C₆ alkyl,

(f) —C(O)—C₁-C₆ alkyl,

(g) optionally substituted:

(1) —C₁-C₅ alkyl-Q⁵,

(2) —C₁-C₅ alkyl-O—C₁-C₃ alkyl-Q⁵,

(3) —C₁-C₃ alkyl-S—C₁-C₃ alkyl-Q⁵,

(4) —C₁-C₅ alkyl-O-Q⁵, or

(5) —C₁-C₅ alkyl-S-Q⁵,

wherein the substituent resides on the alkyl and the substituent is C₁-C₃ alkyl;

(h) -Q⁵;

R¹²⁸ and R^{128′}are each independently selected from the group consisting of:

(a) hydrogen,

(b) CF₃,

(c) CN,

(d) C₁-C₆ alkyl,

(e) -Q⁵,

(f) —O-Q⁵;

(g) —S-Q⁵, and

(h) optionally substituted:

(1) —C₁-C₅ alkyl-Q⁵,

(2) —O—C₁-C₅ alkyl-Q⁵,

(3) —S—C₁-C₅ alkyl-Q⁵,

(4) —C₁-C₃ alkyl-O—C₁-C₃ alkyl-Q⁵,

(5) —C₁-C₃ alkyl-S—C₁-C₃ alkyl-Q⁵,

(6) —C₁-C₅ alkyl-O-Q⁵,

(7) —C₁-C₅ alkyl-S-Q⁵,

wherein the substituent resides on the alkyl and the substituent is C₁-C₃ alkyl, and

R²⁹, R²⁹, R³⁰, R³ and R¹³² are each independently selected from the group consisting of:

(a) hydrogen,

(b) C₁-C₆ alkyl;

or R¹²⁹ and R¹³⁰ or R¹³¹ and R¹³² together with the carbon to which they are attached form a saturated monocyclic carbon ring of 3, 4, 5, 6 or 7 atoms;

Q⁵ is CO₂H, CO₂—C₁-C₄ alkyl, tetrazolyl-5-yl, C(R¹³¹)(R¹³²)(OH), or C(R¹³¹)(R¹³²)(O—C₁-C₄ alkyl);

provided that when X—Y-Z is —S—CR¹²⁸═CR^{128′} then R¹²⁸ and R^{128′} are other than CF₃.

An exemplary phenyl heterocycle that is disclosed in U.S. Pat. No. 6,239,173 is 3-phenyl-4-(4-(methylsulfonyl)phenyl)-2-(2H)-furanone.

Bicycliccarbonyl indole compounds such as those described in U.S. Pat. No. 6,303,628 are useful as Cox-2 selective inhibitors of the present invention. Such bicycliccarbonyl indole compounds have the formula shown below in formula XXV:

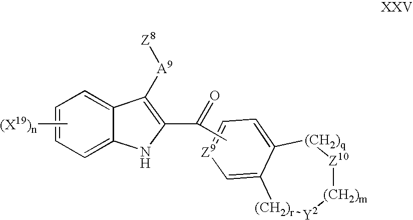

or the pharmaceutically acceptable salts thereof wherein:

A⁹ is C₁-C₆ alkylene or —NR¹³³—;

Z⁸ is C(=L³)R¹³⁴, or SO₂R¹³⁵;

Z⁹ is CH or N;

Z¹⁰ and Y² are independently selected from —CH₂—, O, S and —N—R¹³³;

m is 1, 2 or 3;

q and r are independently 0, 1 or 2;

X¹⁸ is independently selected from halogen, C₁-C₄ alkyl, halo-substituted C₁-C₄ alkyl, hydroxyl, C₁-C₄ alkoxy, halo-substituted C₁-C₄ alkoxy, C₁-C₄ alkylthio, nitro, amino, mono- or di-(C₁-C₄ alkyl)amino and cyano;

n is 0, 1, 2, 3 or 4;

L³ is oxygen or sulfur;

R¹³³ is hydrogen or C₁-C₄ alkyl;

R¹³⁴ is hydroxyl, C₁-C₆ alkyl, halo-substituted C₁-C₆ alkyl, C₁-C₆ alkoxy, halo-substituted C₁-C₆ alkoxy, C₃-C₇ cycloalkoxy, C₁-C₄ alkyl(C₃-C₇ cycloalkoxy), —NR¹³⁶R¹³⁷, C₁-C₄ alkylphenyl-O— or phenyl-O—, said phenyl being optionally substituted with one to five substituents independently selected from halogen, C₁-C₄ alkyl, hydroxyl, C₁-C₄ alkoxy and nitro;

R¹³⁵ is C₁-C₆ alkyl or halo-substituted C₁-C₆ alkyl; and

R¹³⁶ and R¹³⁷ are independently selected from hydrogen, C₁₋₆ alkyl and halo-substituted C₁-C₆ alkyl.

Materials that can serve as a Cox-2 selective inhibitor of the present invention include benzimidazole compounds that are described in U.S. Pat. No. 6,310,079. Such benzimidazole compounds have the formula shown below in formula XXVI:

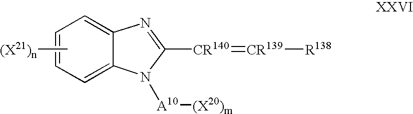

or a pharmaceutically acceptable salt thereof, wherein:

A¹⁰ is heteroaryl selected from

a 5-membered monocyclic aromatic ring having one hetero atom selected from O, S and N and optionally containing one to three N atom(s) in addition to said hetero atom, or

a 6-membered monocyclic aromatic ring having one N atom and optionally containing one to four N atom(s) in addition to said N atom; and said heteroaryl being connected to the nitrogen atom on the benzimidazole through a carbon atom on the heteroaryl ring;

X²⁰ is independently selected from halo, C₁-C₄ alkyl, hydroxyl, C₁-C₄ alkoxy, halo-substituted C₁-C₄ alkyl, hydroxyl-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amonio, N-(C₁-C₄ alkyl)(C₁-C₄ alkanoyl)amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbamoyl, [N-(C₁-C₄ alkyl)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl;

X²¹ is independently selected from halo, C₁-C₄ alkyl, hydroxyl, C₁-C₄ alkoxy, halo-substituted C₁-C₄ alkyl, hydroxyl-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amino, N-(C₁-C₄ alkyl)-N-(C₁-C₄ alkanoyl)amino, N-[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)hydroxyl, cabamoyl, [N-(C₁-C₄ alkyl) amino]carbonyl, [N,N-di(C₁-C₄ alkyl)amino]carbonyl, N-carbomoylamino, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl;

R¹³⁸ is selected from:

hydrogen;

straight or branched C₁-C₄ alkyl optionally substituted with one to three substituent(s) wherein said substituents are independently selected from halo, hydroxyl, C₁-C₄ alkoxy, amino, N-(C₁-C₄ alkyl)amino and N,N-di(C₁-C₄ alkyl)amino;

C₃-C₈ cycloalkyl optionally substituted with one to three substituent(s) wherein said substituents are indepently selected from halo, C₁-C₄ alkyl, hydroxyl, C₁-C₄ alkoxy, amino, N-(C₁-C₄ alkyl)amino and N,N-di(C₁-C₄ alkyl)amino;

C₄-C₈ cycloalkenyl optionally substituted with one to three substituent(s) wherein said substituents are independently selected from halo, C₁-C₄ alkyl, hydroxyl, C₁-C₄ alkoxy, amino, N-(C₁-C₄ alkyl)amino and N,N-di(C₁-C₄ alkyl)amino;

phenyl optionally substituted with one to three substituent(s) wherein said substituents are independently selected from halo, C₁-C₄ alkyl, hydroxyl, C₁-C₄ alkoxy, halo-substituted C₁-C₄ alkyl, □ydroxyl-substituted C₁-C₄ alkyl, (C₁-C₄ alkoxy)C₁-C₄ alkyl, halo-substituted C₁-C₄ alkoxy, amino, N-(C₁-C₄ alkyl)amino, N,N-di(C₁-C₄ alkyl)amino, [N-(C₁-C₄ alkyl)amino]C₁-C₄ alkyl, [N,N-di(C₁-C₄ alkyl)amino]C₁-C₄ alkyl, N-(C₁-C₄ alkanoyl)amino, N-[C₁-C₄ alkyl)(C₁-C₄ alkanoyl)]amino, N-[(C₁-C₄ alkyl)sulfony]amino, N-[(halo-substituted C₁-C₄ alkyl)sulfonyl]amino, C₁-C₄ alkanoyl, carboxy, (C₁-C₄ alkoxy)carbonyl, carbomoyl, [N-(C₁-C₄ alky)amino]carbonyl, [N,N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N-(C₁-C₄ alkyl)amino]sulfonyl and [N,N-di(C₁-C₄ alkyl)amino]sulfonyl; and

heteroaryl selected from:

a 5-membered monocyclic aromatic ring having one hetero atom selected from O, S and N and optionally containing one to three N atom(s) in addition to said hetero atom; or a 6-membered monocyclic aromatic ring having one N atom and optionally containing one to four N atom(s) in addition to said N atom; and

said heteroaryl being optionally substituted with one to three substituent(s) selected from X²⁰;

R¹³⁹ and R¹⁴⁰ are independently selected from:

hydrogen;

halo;

C₁-C₄ alkyl;

phenyl optionally substituted with one to three substituent(s) wherein said substituents are independently selected from halo, C₁-C₄ alkyl, hydroxyl, C₁-C₄ alkoxy, amino, N-(C₁-C₄ alkyl)amino and N,N-di(C₁-C₄ alkyl)amino;

or R¹³⁸ and R¹³⁹ can form, together with the carbon atom to which they are attached, a C₃-C₇ cycloalkyl ring;

m is 0, 1, 2, 3, 4 or 5; and

n is 0, 1, 2, 3 or 4.

Compounds that may be employed as a Cox-2 selective inhibitor of the present invention include indole compounds that are described in U.S. Pat. No. 6,300,363. Such indole compounds have the formula shown below in formula XXVII:

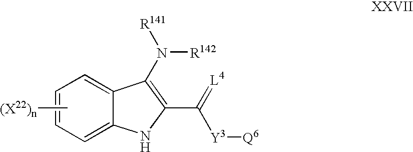

and the pharmaceutically acceptable salts thereof, wherein:

L⁴ is oxygen or sulfur;

Y³ is a direct bond or C₁-C₄ alkylidene;

Q⁶ is:

(a) C₁-C₆ alkyl or halosubstituted C₁-C₆ alkyl, said alkyl being optionally substituted with up to three substituents independently selected from hydroxyl, C₁-C₄ alkoxy, amino and mono- or di-(C₁-C₄ alkyl)amino,

(b) C₃-C₇ cycloalkyl optionally substituted with up to three substituents independently selected from hydroxyl, C₁-C₄ alkyl and C₁-C₄ alkoxy,

(c) phenyl or naphthyl, said phenyl or naphthyl being optionally substituted with up to four substituents independently selected from:

(c-1) halo, C₁-C₄ alkyl, halosubstituted C₁-C₄ alkyl, hydroxyl, C₁-C₄ alkoxy, halosubstituted C₁-C₄ alkoxy, S(O)ₘR¹⁴³, SO₂NH₂, SO₂N(C₁-C₄ alkyl)₂, amino, mono- or di-(C₁-C₄ alkyl)amino, NHSO₂R¹⁴³, NHC(O)R¹⁴³, CN, CO₂H, CO₂(C₁-C₄ alkyl), C₁-C₄ alkyl-OH, C₁-C₄ alkyl-OR¹⁴³, CONH₂, CONH(C₁-C₄ alkyl), CON(C₁-C₄ alkyl)₂ and —O—Y-phenyl, said phenyl being optionally substituted with one or two substituents independently selected from halo, C₁-C₄ alkyl, CF₃, hydroxyl, OR¹⁴³, S(O)ₘR¹⁴³, amino, mono- or di-(C₁-C₄ alkyl)amino and CN;

(d) a monocyclic aromatic group of 5 atoms, said aromatic group having one heteroatom selected from O, S and N and optionally containing up to three N atoms in addition to said heteroatom, and said aromatic group being substituted with up to three substitutents independently selected from:

(d-1) halo, C₁-C₄ alkyl, halosubstituted C₁-C₄ alkyl, hydroxyl, C₁-C₄ alkoxy, halosubstituted C₁-C₄ alkoxy, C₁-C₄ alkyl-OH, S(O)ₘR¹⁴³, SO₂ NH₂, SO₂N(C₁-C₄ alkyl)₂, amino, mono- or di-(C₁-C₄ alkyl)amino, NHSO₂R¹⁴³, NHC(O)R¹⁴³, CN, CO₂H, CO₂(C₁-C₄ alkyl), C₁-C₄ alkyl-OR¹⁴³, CONH₂, CONH(C₁-C₄ alkyl), CON(C₁-C₄ alkyl)₂, phenyl, and mono-, di- or tri-substituted phenyl wherein the substituent is independently selected from halo, CF₃, C₁-C₄ alkyl, hydroxyl, C₁-C₄ alkoxy, OCF₃, SR¹⁴³, SO₂ CH₃, SO₂ NH₂, amino, C₁₋₄ alkylamino and NHSO₂R¹⁴³;

(e) a monocyclic aromatic group of 6 atoms, said aromatic group having one heteroatom which is N and optionally containing up to three atoms in addition to said heteroatom, and said aromatic group being substituted with up to three substituents independently selected from the above group (d-1);

R¹⁴¹ is hydrogen or C₁-C₆ alkyl optionally substituted with a substituent selected independently from hydroxyl, OR¹⁴³, nitro, amino, mono- or di-(C₁-C₄ alkyl)amino, CO₂H, CO₂ (C₁-C₄ alkyl), CONH₂, CONH(C₁-C₄ alkyl) and CON(C₁-C₄ alkyl)₂;

R¹⁴² is:

(a) hydrogen,

(b) C₁-C₄ alkyl,

(c) C(O)R¹⁴⁵,

wherein R¹⁴⁵ is selected from:

(c-1) C₁-C₂₂ alkyl or C₂-C₂₂ alkenyl, said alkyl or alkenyl being optionally substituted with up to four substituents independently selected from:

(c-1-1) halo, hydroxyl, OR¹⁴³, S(O)ₘR¹⁴³, nitro, amino, mono- or di-(C₁-C₄ alkyl)amino, NHSO₂R¹⁴³, CO₂H, CO₂(C₁-C₄ alkyl), CONH₂, CONH(C₁-C₄ alkyl), CON(C₁-C₄ alkyl)₂, OC(O)R¹⁴³, thienyl, naphthyl and groups of the following formulas:

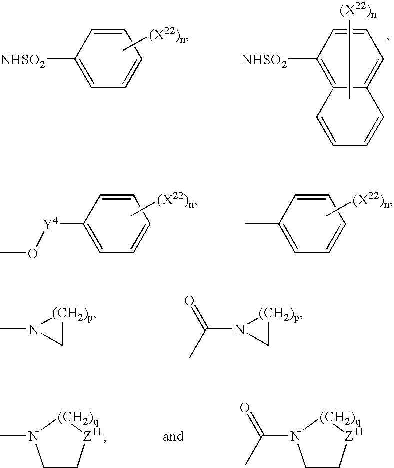

(c-2) C₁-C₂₂ alkyl or C₂-C₂₂ alkenyl, said alkyl or alkenyl being optionally substituted with five to forty-five halogen atoms,

(c-3) —Y⁵—C₃-C₇ cycloalkyl or —Y⁵—C₃-C₇ cycloalkenyl, said cycloalkyl or cycloalkenyl being optionally substituted with up to three substituent independently selected from:

(c-3-1) C₁-C₄ alkyl, hydroxyl, OR¹⁴³ S(O)ₘR¹⁴³, amino, mono or di-(C₁-C₄ alkyl)amino, CONH₂, CONH(C₁-C₄ alkyl) and CON(C₁-C₄ alkyl)₂,

(c-4) phenyl or naphthyl, said phenyl or naphthyl being optionally substituted with up to seven (preferably up to seven) substituents independently selected from:

(c-4-1) halo, C₁-C₈ alkyl, C₁-C₄ alkyl-OH, hydroxyl, C₁-C₈ alkoxy, halosubstituted C₁-C₈ alkyl, halosubstituted C₁-C₈ alkoxy, CN, nitro, S(O)ₘR¹⁴³, SO₂ NH₂, SO₂ NH(C₁-C₄ alkyl), SO₂N(C₁-C₄ alkyl)₂, amino, C₁-C₄ alkylamino, di-(C₁-C₄ alkyl)amino, CONH₂, CONH(C₁-C₄ alkyl), CON(C₁-C₄ alkyl)₂, OC(O)R¹⁴³, and phenyl optionally substituted with up to three substituents independently selected from halo, C₁-C₄ alkyl, hydroxyl, OCH₃, CF₃, OCF₃, CN, nitro, amino, mono- or di-(C₁-C₄ alkyl)amino, CO₂H, CO₂ (C₁-C₄ alkyl) and CONH₂,

(c-5) a monocyclic aromatic group as defined in (d) and (e) above, said aromatic group being optionally substituted with up to three substituents independently selected from:

(c-5-1) halo, C₁-C₈ alkyl, C₁-C₄ alkyl-OH, hydroxyl, C₁-C₈ alkoxy, CF₃, OCF₃, CN, nitro, S(O)ₘR⁴³, amino, mono- or di-(C₁-C₄ alkyl)amino, CONH₂, CONH(C₁-C₄ alkyl), CON(C₁-C₄ alkyl)₂, CO₂H and CO₂ (C₁-C₄ alkyl), and —Y-phenyl, said phenyl being optionally substituted with up to three substituents independently selected halogen, C₁-C₄ alkyl, hydroxyl, C₁-C₄ alkoxy, CF₃, OCF₃, CN, nitro, S(O)ₘR¹⁴³, amino, mono- or di-(C₁-C₄ alkyl)amino, CO₂H, CO₂ (C₁-C₄ alkyl), CONH₂, CONH(C₁-C₄ alkyl) and CON(C₁-C₄ alkyl)₂,

(c-6) a group of the following formula:

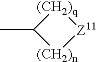

X²² is halo, C₁-C₄ alkyl, hydroxyl, C₁-C₄ alkoxy, halosubstitutued C₁-C₄alkoxy, S(O)ₘR¹⁴³, amino, mono- or di-(C₁-C₄ alkyl)amino, NHSO₂R¹⁴³, nitro, halosubstitutued C₁-C₄ alkyl, CN, CO₂H, CO₂ (C₁-C₄ alkyl), C₁-C₄ alkyl-OH, C₁-C₄ alkylOR¹⁴³, CONH₂, CONH(C₁-C₄ alkyl) or CON(C₁-C₄ alkyl)₂;

R¹⁴³ is C₁-C₄ alkyl or halosubstituted C₁-C₄ alkyl;

m is 0, 1 or 2; n is 0, 1, 2 or 3; p is 1, 2, 3, 4 or 5; q is 2 or 3;

Z¹¹ is oxygen, sulfur or NR¹⁴⁴; and

R₁₄₄ is hydrogen, C₁-C₆ alkyl, halosubstitutued C₁-C₄ alkyl or —Y⁵-phenyl, said phenyl being optionally substituted with up to two substituents independently selected from halo, C₁-C₄ alkyl, hydroxyl, C₁-C₄ alkoxy, S(O)ₘR¹⁴³, amino, mono- or di-(C₁-C₄ alkyl)amino, CF₃, OCF₃, CN and nitro;

with the proviso that a group of formula —Y⁵-Q is not methyl or ethyl when X²² is hydrogen;

L⁴ is oxygen;

R¹⁴¹ is hydrogen; and

R¹⁴² is acetyl.

Aryl phenylhydrazides that are described in U.S. Pat. No. 6,077,869 can serve as Cox-2 selective inhibitors of the present invention. Such aryl phenylhydrazides have the formula shown below in formula XXVIII:

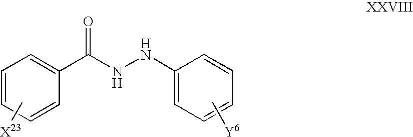

wherein:

X²³ and Y⁶ are selected from hydrogen, halogen, alkyl, nitro, amino, hydroxy, methoxy and methylsulfonyl;

or a pharmaceutically acceptable salt thereof.

Materials that can serve as a Cox-2 selective inhibitor of the present invention include 2-aryloxy, 4-aryl furan-2-ones that are described in U.S. Pat. No. 6,140,515. Such 2-aryloxy, 4-aryl furan-2-ones have the formula shown below in formula XXIX:

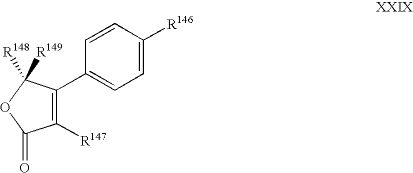

or a pharmaceutical salt thereof, wherein:

R¹⁴⁶ is selected from the group consisting of SCH₃, —S(O)₂ CH₃ and —S(O)₂ NH₂;

R¹⁴⁷ is selected from the group consisting of OR¹⁵⁰, mono or di-substituted phenyl or pyridyl wherein the substituents are selected from the group consisting of methyl, chloro and F;

R¹⁵⁰ is unsubstituted or mono or di-substituted phenyl or pyridyl wherein the substituents are selected from the group consisting of methyl, chloro and F;

R¹⁴⁸ is H, C₁-C₄ alkyl optionally substituted with 1 to 3 groups of F, Cl or Br; and

R¹⁴⁹ is H, C₁-C₄ alkyl optionally substituted with 1 to 3 groups of F, Cl or Br, with the proviso that R¹⁴⁸ and R¹⁴⁹ are not the same.

Materials that can serve as a Cox-2 selective inhibitor of the present invention include bisaryl compounds that are described in U.S. Pat. No. 5,994,379. Such bisaryl compounds have the formula shown below in formula XXX:

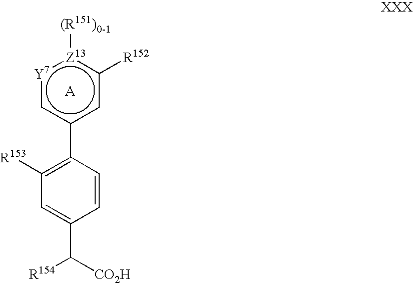

or a pharmaceutically acceptable salt, ester or tautomer thereof, wherein:

Z¹³ is C or N;

when Z¹³ is N, R¹⁵¹ represents H or is absent, or is taken in conjunction with R¹⁵² as described below:

when Z¹³ is C, R¹⁵¹ represents H and R¹⁵² is a moiety which has the following characteristics:

(a) it is a linear chain of 3-4 atoms containing 0-2 double bonds, which can adopt an energetically stable transoid configuration and if a double bond is present, the bond is in the trans configuration,

(b) it is lipophilic except for the atom bonded directly to ring A, which is either lipophilic or non-lipophilic, and

(c) there exists an energetically stable configuration planar with ring A to within about 15 degrees;

or R¹⁵¹ and R¹⁵² are taken in combination and represent a 5- or 6-membered aromatic or non-aromatic ring D fused to ring A, said ring D containing 0-3 heteroatoms selected from O, S and N;

said ring D being lipophilic except for the atoms attached directly to ring A, which are lipophilic or non-lipophilic, and said ring D having available an energetically stable configuration planar with ring A to within about 15 degrees;

said ring D further being substituted with 1 R^{a} group selected from the group consisting of: C₁-C₂ alkyl, —OC₁-C₂ alkyl, —NHC, —C₂ alkyl, —N(C₁-C₂ alkyl)₂, —C(O)C₁-C₂ alkyl, —S—C₁-C₂ alkyl and —C(S)C₁-C₂ alkyl;

Y⁷ represents N, CH or C—OC₁-C₃ alkyl, and when Z¹³ is N, Y⁷ can also represent a carbonyl group;

R¹⁵³ represents H, Br, Cl or F; and

R¹⁵⁴ represents H or CH₃.

Compounds useful as Cox-2 selective inhibitors of the present invention include 1,5-diarylpyrazoles that are described in U.S. Pat. No. 6,028,202. Such 1,5-diarylpyrazoles have the formula shown below in formula XXXI:

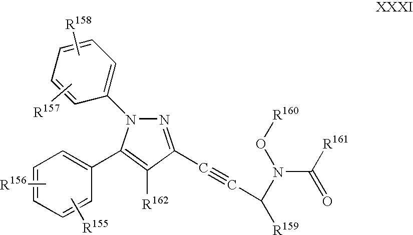

wherein:

R¹⁵⁵, R¹⁵⁶, R¹⁵⁷, and R¹⁵⁸ are independently selected from the groups consisting of hydrogen, C₁-C₅ alkyl, C₁-C₅ alkoxy, phenyl, halo, hydroxyl, C₁-C₅ alkylsulfonyl, C₁-C₅ alkylthio, trihaloC₁-C₅ alkyl, amino, nitro and 2-quinolinylmethoxy;

R¹⁵⁹ is hydrogen, C₁-C₅ alkyl, trihaloC₁-C₅ alkyl, phenyl, substituted phenyl where the phenyl substitutents are halogen, C₁-C₅ alkoxy, trihaloC₁-C₅ alkyl or nitro or R¹⁵⁹ is heteroaryl of 5-7 ring members where at least one of the ring members is nitrogen, sulfur or oxygen;

R¹⁶⁰ is hydrogen, C₁-C₅ alkyl, phenyl C₁-C₅ alkyl, substituted phenyl C₁-C₅ alkyl where the phenyl substitutents are halogen, C₁-C₅ alkoxy, trihaloC₁-C₅ alkyl or nitro, or R¹⁶⁰ is C₁-C₅ alkoxycarbonyl, phenoxycarbonyl, substituted phenoxycarbonyl where the phenyl substitutents are halogen, C₁-C₅ alkoxy, trihaloC₁-C₅ alkyl or nitro;

R¹⁶¹ is C₁-C₁₀ alkyl, substituted C₁-C₁₀ alkyl where the substituents are halogen, trihaloC₁-C₅ alkyl, C₁-C₅ alkoxy, carboxy, C₁-C₅ alkoxycarbonyl, amino, C₁-C₅ alkylamino, diC₁-C₅ alkylamino, diC₁-C₅ alkylaminoC₁-C₅ alkylamino, C₁-C₅ alkylaminoC₁-C₅ alkylamino or a heterocycle containing 4-8 ring atoms where one more of the ring atoms is nitrogen, oxygen or sulfur, where said heterocycle may be optionally substituted with C₁-C₅ alkyl; or R¹⁶¹ is phenyl, substituted phenyl (where the phenyl substitutents are one or more of C₁-C₅ alkyl, halogen, C₁-C₅ alkoxy, trihaloC₁-C₅ alkyl or nitro), or R¹⁶¹ is heteroaryl having 5-7 ring atoms where one or more atoms are nitrogen, oxygen or sulfur, fused heteroaryl where one or more 5-7 membered aromatic rings are fused to the heteroaryl; or

R¹⁶¹ is NR¹⁶³R¹⁶⁴ where R¹⁶³ and R¹⁶⁴ are independently selected from hydrogen and C₁₋₅ alkyl or R¹⁶³ and R¹⁶⁴ may be taken together with the depicted nitrogen to form a heteroaryl ring of 5-7 ring members where one or more of the ring members is nitrogen, sulfur or oxygen where said heteroaryl ring may be optionally substituted with C₁-C₅ alkyl; R¹⁶² is hydrogen, C₁-C₅ alkyl, nitro, amino, and halogen;

and pharmaceutically acceptable salts thereof.

Materials that can serve as a Cox-2 selective inhibitor of the present invention include 2-substituted imidazoles that are described in U.S. Pat. No. 6,040,320. Such 2-substituted imidazoles have the formula shown below in formula XXXII:

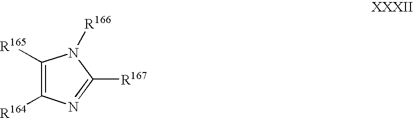

wherein:

R¹⁵⁴ is phenyl, heteroaryl wherein the heteroaryl contains 5 to 6 ring atoms, or

substituted phenyl;

wherein the substituents are independently selected from one or members of the group consisting of C₁₋₅ alkyl, halogen, nitro, trifluoromethyl and nitrile;

R¹⁶⁵ is phenyl, heteroaryl wherein the heteroaryl contains 5 to 6 ring atoms,

substituted heteroaryl;

wherein the substituents are independently selected from one or more members of the group consisting of C₁-C₅ alkyl and halogen, or

substituted phenyl,

wherein the substituents are independently selected from one or members of the group consisting of C₁-C₅ alkyl, halogen, nitro, trifluoromethyl and nitrile;

R¹⁶⁶ is hydrogen, 2-(trimethylsilyl)ethoxymethyl), C₁-C₅ alkoxycarbonyl, aryloxycarbonyl, arylC₁-C₅ alkyloxycarbonyl, arylC₁-C₅ alkyl, phthalimidoC₁-C₅ alkyl, aminoC₁-C₅ alkyl, diaminoC₁-C₅ alkyl, succinimidoC₁-C₅ alkyl, C₁-C₅ alkylcarbonyl, arylcarbonyl, C₁-C₅ alkylcarbonylC₁-C₅ alkyl, aryloxycarbonylC₁-C₅ alkyl, heteroarylC₁-C₅ alkyl where the heteroaryl contains 5 to 6 ring atoms, or substituted arylC₁-C₅ alkyl, wherein the aryl substituents are independently selected from one or more members of the group consisting of C₁-C₅ alkyl, C₁-C₅ alkoxy, halogen, amino, C₁-C₅ alkylamino, and diC₁-C₅ alkylamino;

R¹⁶⁷ is (A¹¹)ₙ—(CH¹⁶⁵)_{q}—X²⁴ wherein:

A¹¹ is sulfur or carbonyl;

n is 0 or 1;

q is 0-9;

X²⁴ is selected from the group consisting of hydrogen, hydroxyl, halogen, vinyl, ethynyl, C₁-C₅ alkyl, C₃-C₇ cycloalkyl, C₁-C₅ alkoxy, phenoxy, phenyl, arylC₁-C₅ alkyl, amino, C₁-C₅ alkylamino, nitrile, phthalimido, amido, phenylcarbonyl, C₁-C₅ alkylaminocarbonyl, phenylaminocarbonyl, arylC₁-C₅ alkylaminocarbonyl, C₁-C₅ alkylthio, C₁-C₅ alkylsulfonyl, phenylsulfonyl,

substituted sulfonamido,

wherein the sulfonyl substituent is selected from the group consisting of C₁-C₅ alkyl, phenyl, araC₁-C₅ alkyl, thienyl, furanyl, and naphthyl; substituted vinyl,

wherein the substituents are independently selected from one or members of the group consisting of fluorine, bromine, chlorine and iodine, substituted ethynyl,

wherein the substituents are independently selected from one or more members of the group consisting of fluorine, bromine chlorine and iodine,

substituted C₁-C₅ alkyl,

wherein the substituents are selected from the group consisting of one or more C₁-C₅ alkoxy, trihaloalkyl, phthalimido and amino,

substituted phenyl,

wherein the phenyl substituents are independently selected from one or more members of the group consisting of C₁-C₅ alkyl, halogen and C₁-C₅ alkoxy,

substituted phenoxy,

wherein the phenyl substituents are independently selected from one or more members of the group consisting of C₁-C₅ alkyl, halogen and C₁-C₅ alkoxy,

substituted C₁-C₅ alkoxy,

wherein the alkyl substituent is selected from the group consisting of phthalimido and amino,

substituted arylC₁-C₅ alkyl,

wherein the alkyl substituent is hydroxyl,

substituted arylC₁-C₅ alkyl,

wherein the phenyl substituents are independently selected from one or more members of the group consisting of C₁-C₅ alkyl, halogen and C₁-C₅ alkoxy,

substituted amido,

wherein the carbonyl substituent is selected from the group consisting of C₁-C₅ alkyl, phenyl, arylC₁-C₅ alkyl, thienyl, furanyl, and naphthyl,

substituted phenylcarbonyl,

wherein the phenyl substituents are independently selected from one or members of the group consisting of C₁-C₅ alkyl, halogen and C₁-C₅ alkoxy,

substituted C₁-C₅ alkylthio,

wherein the alkyl substituent is selected from the group consisting of hydroxyl and phthalimido,

substituted C₁-C₅ alkylsulfonyl,

wherein the alkyl substituent is selected from the group consisting of hydroxyl and phthalimido,

substituted phenylsulfonyl,

wherein the phenyl substituents are independently selected from one or members of the group consisting of bromine, fluorine, chlorine, C₁-C₅ alkoxy and trifluoromethyl,

with the proviso:

if A¹¹ is sulfur and X²⁴ is other than hydrogen, C₁-C₅ alkylaminocarbonyl, phenylaminocarbonyl, arylC₁-C₅ alkylaminocarbonyl, C₁-C₅ alkylsulfonyl or phenylsulfonyl, then q must be equal to or greater than 1;

if A¹¹ is sulfur and q is 1, then X²⁴ cannot be C₁-C₂ alkyl;

if A¹¹ is carbonyl and q is 0, then X²⁴ cannot be vinyl, ethynyl, C₁-C₅ alkylaminocarbonyl, phenylaminocarbonyl, arylC₁-C₅ alkylaminocarbonyl, C₁-C₅ alkylsulfonyl or phenylsulfonyl;

if A¹¹ is carbonyl, q is 0 and X²⁴ is H, then R¹⁶⁶ is not 2-(trimethylsilyl)ethoxymethyl;

if n is 0 and q is 0, then X²⁴ cannot be hydrogen;

and pharmaceutically acceptable salts thereof.

Materials that can serve as a Cox-2 selective inhibitor of the present invention include 1,3- and 2,3-diarylcycloalkano and cycloalkeno pyrazoles that are described in U.S. Pat. No. 6,083,969. Such 1,3- and 2,3-diarylpyrazole compounds have the general formulas shown below in formulas XXXIII and XXXIV:

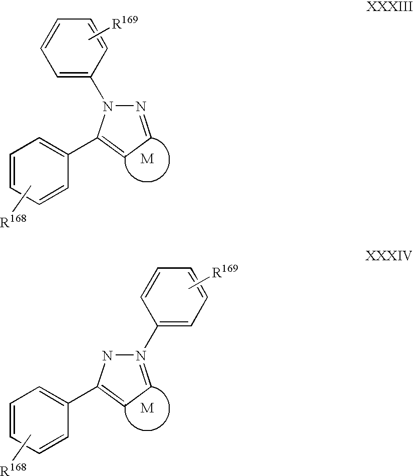

wherein:

R¹⁶⁸ and R¹⁶⁹ are independently selected from the group consisting of hydrogen, halogen, (C₁-C₆)alkyl, (C₁-C₆)alkoxy, nitro, amino, □ydroxyl, trifluoro, —S(C₁-C₆)alkyl, —SO(C₁-C₆)alkyl and —SO₂ (C₁-C₆)alkyl; and

the fused moiety M is a group selected from the group consisting of an optionally substituted cyclohexyl and cycloheptyl group having the formulae:

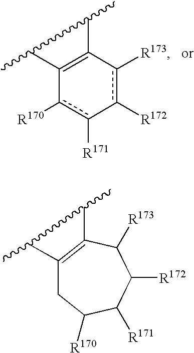

wherein:

R¹⁷⁰ is selected from the group consisting of hydrogen, halogen, hydroxyl and carbonyl;

or R¹⁷⁰ and R¹⁷¹ taken together form a moiety selected from the group consisting of —OCOCH₂—, —ONH(CH₃)COCH₂—, —OCOCH═ and —O—;

R¹⁷¹ and R¹⁷² are independently selected from the group consisting of hydrogen, halogen, hydroxyl, carbonyl, amino, (C₁-C₆)alkyl, (C₁-C₆)alkoxy, ═NOH, —NR¹⁷⁴R¹⁷⁵, —OCH₃, —OCH₂CH₃, —OSO₂NHCO₂CH₃, ═CHCO₂CH₂CH₃, —CH₂CO₂H, —CH₂CO₂ CH₃, —CH₂CO₂CH₂ CH₃, —CH₂CON(CH₃)₂, —CH₂CO₂NHCH₃, —CHCHCO₂CH₂CH₃, —OCON(CH₃)OH, —C(COCH₃)₂, di(C₁-C₆)alkyl and di(C₁-C₆)alkoxy;

R¹⁷³ is selected from the group consisting of hydrogen, halogen, hydroxyl, carbonyl, amino, (C₁-C₆)alkyl, (C₁-C₆)alkoxy and optionally substituted carboxyphenyl, wherein substituents on the carboxyphenyl group are selected from the group consisting of halogen, hydroxyl, amino, (C₁-C₆)alkyl and (C₁-C₆)alkoxy;

or R¹⁷² and R¹⁷³ taken together form a moiety selected from the group consisting of —O— and

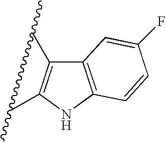

R¹⁷⁴ is selected from the group consisting of hydrogen, OH, —OCOCH₃, —COCH₃ and (C₁-C₆)alkyl; and

R¹⁷⁵ is selected from the group consisting of hydrogen, OH, —OCOCH₃, —COCH₃, (C₁-C₆)alkyl, —CONH₂ and —SO₂CH₃;

with the proviso that

if M is a cyclohexyl group, then R¹⁷⁰ through R¹⁷³ may not all be hydrogen; and

pharmaceutically acceptable salts, esters and pro-drug forms thereof.

Esters derived from indolealkanols and novel amides derived from indolealkylamides that are described in U.S. Pat. No. 6,306,890 can serve as Cox-2 selective inhibitors of the present invention. Such compounds have the general formula shown below in formula XXXV:

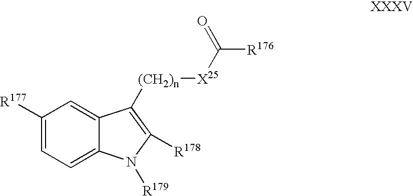

wherein:

R¹⁷⁶ is C₁-C₆ alkyl, C₁-C₆ branched alkyl, C₄-C₈ cycloalkyl, C₁-C₆ hydroxyalkyl, branched C₁-C₆ hydroxyalkyl, hydroxyl substituted C₄-C₈ aryl, primary, secondary or tertiary C₁-C₆ alkylamino, primary, secondary or tertiary branched C₁-C₆ alkylamino, primary, secondary or tertiary C₄-C₈ arylamino, C₁-C₆ alkylcarboxylic acid, branched C₁-C₆ alkylcarboxylic acid, C₁-C₆ alkylester, branched C₁-C₆ alkylester, C₄-C₈ aryl, C₄-C₈ arylcarboxylic acid, C₄-C₈ arylester, C₄-C₈ aryl substituted C₁-C₆ alkyl, C₄-C₈ heterocyclic alkyl or aryl with O, N or S in the ring, alkyl-substituted or aryl-substituted C₄-C₈ heterocyclic alkyl or aryl with O, N or S in the ring, or halo-substituted versions thereof, where halo is chloro, bromo, fluoro or iodo;

R¹⁷⁷ is C₁-C₆ alkyl, C₁-C₆ branched alkyl, C₄-C₈ cycloalkyl, C₄-C₈ aryl, C₄-C₈ aryl-substituted C₁-C₆ alkyl, C₁-C₆ alkoxy, C₁-C₆ branched alkoxy, C₄-C₈ aryloxy, or halo-substituted versions thereof or R¹⁷⁷ is halo where halo is chloro, fluoro, bromo, or iodo;

R¹⁷⁸ is hydrogen, C₁-C₆ alkyl or C₁-C₆ branched alkyl;

R¹⁷⁹ is C₁-C₆ alkyl, C₄-C₈ aroyl, C₄-C₈ aryl, C₄-C₈ heterocyclic alkyl or aryl with O, N or S in the ring, C₄-C₈ aryl-substituted C₁-C₆ alkyl, alkyl-substituted or aryl-substituted C₄-C₈ heterocyclic alkyl or aryl with O, N or S in the ring, alkyl-substituted C₄-C₈ aroyl, or alkyl-substituted C₄-C₈ aryl, or halo-substituted versions thereof where halo is chloro, bromo, or iodo;

n is 1, 2, 3, or 4; and

X²⁵ is O, NH, or N—R¹⁸⁰, where R¹⁸⁰ is C₁-C₆ or C₁-C₆ branched alkyl.

Materials that can serve as a Cox-2 selective inhibitor of the present invention include pyridazinone compounds that are described in U.S. Pat. No. 6,307,047. Such pyridazinone compounds have the formula shown below in formula XXXVI:

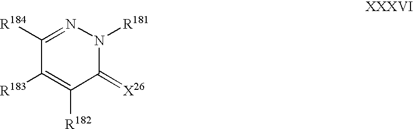

or a pharmaceutically acceptable salt, ester, or prodrug thereof, wherein:

X²⁶ is selected from the group consisting of O, S, —NR¹⁸⁵, —NOR^{a}, and —NNR^{b}R^{c};

R¹⁸⁵ is selected from the group consisting of alkenyl, alkyl, aryl, arylalkyl, cycloalkenyl, cycloalkenylalkyl, cycloalkyl, cycloalkylalkyl, heterocyclic, and heterocyclic alkyl;

R^{a}, R^{b}, and R^{c} are independently selected from the group consisting of alkyl, aryl, arylalkyl, cycloalkyl, and cycloalkylalkyl;

R¹⁸¹ is selected from the group consisting of alkenyl, alkoxy, alkoxyalkyl, alkoxyiminoalkoxy, alkyl, alkylcarbonylalkyl, alkylsulfonylalkyl, alkynyl, aryl, arylalkenyl, arylalkoxy, arylalkyl, arylalkynyl, arylhaloalkyl, arylhydroxyalkyl, aryloxy, aryloxyhaloalkyl, aryloxyhydroxyalkyl, arylcarbonylalkyl, carboxyalkyl, cyanoalkyl, cycloalkenyl, cycloalkenylalkyl, cycloalkyl, cycloalkylalkyl, cycloalkylidenealkyl, haloalkenyl, haloalkoxyhydroxyalkyl, haloalkyl, haloalkynyl, heterocyclic, heterocyclic alkoxy, heterocyclic alkyl, heterocyclic oxy, hydroxyalkyl, hydroxyiminoalkoxy, —(CH₂)ₙC(O)R¹⁸⁶, —(CH₂)ₙCH(OH)R¹⁸⁶, —(CH₂)ₙC(NOR^{d})R¹⁸⁶, —(CH₂)ₙCH(NOR^{d})R¹⁸⁶, —(CH₂)ₙCH(NR^{d}R^{e})R¹⁸⁶, —R¹⁸⁷R¹⁸⁸, —(CH₂)ₙC≡CR¹⁸⁸, —(CH₂)ₙ[CH(CX^{26′}₃)]ₘ(CH₂)ₚR¹⁸⁸, —(CH₂)ₙ(CX²⁶′₂)ₘ(CH₂)ₚR¹⁸⁸, and —(CH₂)ₙ(CHX²⁶′)ₘ(CH₂)ₘR¹⁸⁸;

R¹⁸⁶ is selected from the group consisting of hydrogen, alkenyl, alkyl, alkynyl, aryl, arylalkyl, cycloalkenyl, cycloalkyl, haloalkenyl, haloalkyl, haloalkynyl, heterocyclic, and heterocyclic alkyl;

R¹⁸⁷ is selected from the group consisting of alkenylene, alkylene, halo-substituted alkenylene, and halo-substituted alkylene;

R¹⁸⁸ is selected from the group consisting of hydrogen, alkenyl, alkyl, alkynyl, aryl, arylalkyl, cycloalkyl, cycloalkenyl, haloalkyl, heterocyclic, and heterocyclic alkyl;

R^{d} and R^{e} are independently selected from the group consisting of hydrogen, alkenyl, alkyl, alkynyl, aryl, arylalkyl, cycloalkenyl, cycloalkyl, haloalkyl, heterocyclic, and heterocyclic alkyl;

X²⁶ is halogen;

m is an integer from 0-5;

n is an integer from 0-10;

p is an integer from 0-10;

R¹⁸², R¹⁸³, and R¹⁸⁴ are independently selected from the group consisting of hydrogen, alkenyl, alkoxyalkyl, alkoxyiminoalkoxy, alkoxyiminoalkyl, alkyl, alkynyl, alkylcarbonylalkoxy, alkylcarbonylamino, alkylcarbonylaminoalkyl, aminoalkoxy, aminoalkylcarbonyloxyalkoxy aminocarbonylalkyl, aryl, arylalkenyl, arylalkyl, arylalkynyl, carboxyalkylcarbonyloxyalkoxy, cyano, cycloalkenyl, cycloalkyl, cycloalkylidenealkyl, haloalkenyloxy, haloalkoxy, haloalkyl, halogen, heterocyclic, hydroxyalkoxy, hydroxyiminoalkoxy, hydroxyiminoalkyl, mercaptoalkoxy, nitro, phosphonatoalkoxy, Y⁸, and Z¹⁴; provided that one of R¹⁸², R¹⁸³, or R¹⁸⁴ must be Z¹⁴, and further provided that only one of R¹⁸², R¹⁸³, or R¹⁸⁴ is Z¹⁴;

Z¹⁴ is selected from the group consisting of:

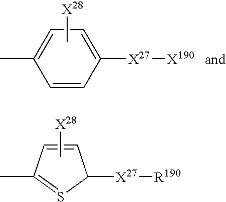

X²⁷ is selected from the group consisting of S(O)₂, S(O)(NR¹⁹¹), S(O), Se(O)₂, P(O)(OR¹⁹²), and P(O)(NR¹⁹³R¹⁹⁴);

X²⁸ is selected from the group consisting of hydrogen, alkenyl, alkyl, alkynyl and halogen;

R¹⁹⁰ is selected from the group consisting of alkenyl, alkoxy, alkyl, alkylamino, alkylcarbonylamino, alkynyl, amino, cycloalkenyl, cycloalkyl, dialkylamino, —NHNH₂, and —NCHN(R¹⁹¹)R¹⁹²;

R¹⁹¹, R¹⁹², R¹⁹³, and R¹⁹⁴ are independently selected from the group consisting of hydrogen, alkyl, and cycloalkyl, or R¹⁹³ and R¹⁹⁴ can be taken together, with the nitrogen to which they are attached, to form a 3-6 membered ring containing 1 or 2 heteroatoms selected from the group consisting of O, S, and NR¹⁸⁸;

Y⁸ is selected from the group consisting of —OR¹⁹⁵, —SR¹⁹⁵, —C(R¹⁹⁷)(R¹⁹⁸)R¹⁹⁵, —C(O)R¹⁹⁵, —C(O)OR¹⁹⁵, —N(R¹⁹⁷)C(O)R¹⁹⁵, —NC(R¹⁹⁷)R¹⁹⁵, and —N(R¹⁹⁷)R¹⁹⁵;

R¹⁹⁵ is selected from the group consisting of hydrogen, alkenyl, alkoxyalkyl, alkyl, alkylthioalkyl, alkynyl, cycloalkenyl, cycloalkenylalkyl, cycloalkyl, cycloalkylalkyl, aryl, arylalkyl, heterocyclic, heterocyclic alkyl, hydroxyalkyl, and NR¹⁹⁹R²⁰⁰; and

R⁹⁷, R¹⁹⁸, R¹⁹⁹, and R²⁰⁰ are independently selected from the group consisting of hydrogen, alkenyl, alkoxy, alkyl, cycloalkenyl, cycloalkyl, aryl, arylalkyl, heterocyclic, and heterocyclic alkyl.

Benzosulphonamide derivatives that are described in U.S. Pat. No. 6,004,948 are useful as Cox-2 selective inhibitors of the present invention. Such benzosulphonamide derivatives have the formula shown below in formula XXXVII:

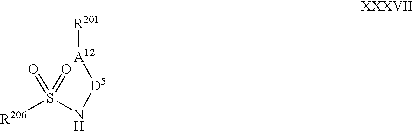

wherein:

A¹² denotes oxygen, sulphur or NH;

R²⁰¹ denotes a cycloalkyl, aryl or heteroaryl group optionally mono- or polysubstituted by halogen, alkyl, CF₃ or alkoxy;

D⁵ denotes a group of formula XXXVIII or XXXIX:

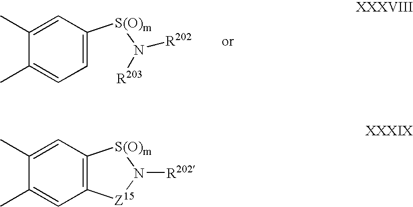

R²⁰² and R²⁰³ independently of each other denote hydrogen, an optionally polyfluorinated alkyl radical, an aralkyl, aryl or heteroaryl radical or a radical (CH₂)ₙ—X²⁹; or

R²⁰² and R²⁰³ together with the N-atom denote a three- to seven-membered, saturated, partially or totally unsaturated heterocycle with one or more heteroatoms N, O, or S, which may optionally be substituted by oxo, an alkyl, alkylaryl or aryl group or a group (CH₂)ₙ—X²⁹, R²⁰²′ denotes hydrogen, an optionally polyfluorinated alkyl group, an aralkyl, aryl or heteroaryl group or a group (CH₂)ₙ—X²⁹,

wherein:

X²⁹ denotes halogen, NO₂, —OR²⁰⁴, —COR²⁰⁴, —CO₂R²⁰⁴, —OCO₂R²⁰⁴, —CN, —CONR²⁰⁴OR²⁰⁵, —CONR²⁰⁴R²⁰⁵, —SR²⁰⁴, —S(O)R²⁰⁴, —S(O)₂R²⁰⁴, —NR²⁰⁴R²⁰⁵. —NHC(O)R²⁰⁴, —NHS(O)₂R²⁰⁴;

Z¹⁵ denotes —CH₂—, —CH₂—CH₂—, —CH₂—CH₂—CH₂—, —CH₂—CH═CH—, —CH═CH—CH₂—, —CH₂—CO—, —CO—CH₂—, —NHCO—, —CONH—, —NHCH₂—, —CH₂ NH—, —N═CH—, —NHCH—, —CH₂—CH₂—NH—, —CH═CH—, >N—R²⁰³, >C═O, >S(O)ₘ;

R²⁰⁴ and R²⁰⁵ independently of each other denote hydrogen, alkyl, aralkyl or aryl;

n is an integer from 0 to 6;

R²⁰⁶ is a straight-chained or branched C₁-C₄ alkyl group which may optionally be mono- or polysubstituted by halogen or alkoxy, or R²⁰⁶ denotes CF₃; and

m denotes an integer from 0 to 2;

with the proviso that A¹² does not represent 0 if R²⁰⁶ denotes CF₃;

and the pharmaceutically acceptable salts thereof.

Materials that can serve as Cox-2 selective inhibitors of the present invention include methanesulfonyl-biphenyl derivatives that are described in U.S. Pat. No. 6,583,321. Such methanesulfonyl-biphenyl derivatives have the formula shown below in formula XXXX:

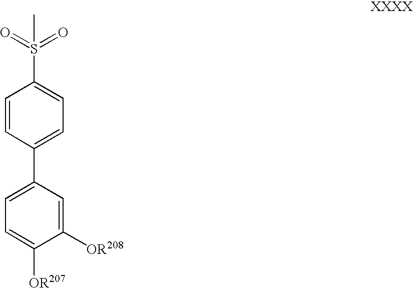

wherein:

R²⁰⁷ and R²⁰⁸ are respectively a hydrogen;

C₁-C₄-alkyl substituted or not substituted by halogens;

C₃-C₇-cycloalkyl;

C₁-C₅-alkyl containing 1-3 ether bonds and/or an aryl substitute;

substituted or not substituted phenyl;

or substituted or not substituted five or six ring-cycled heteroaryl containing more than one hetero atoms selected from a group consisting of nitrogen, sulfur, and oxygen (wherein phenyl or heteroaryl can be one- or multi-substituted by a substituent selected from a group consisting of hydrogen, methyl, ethyl, and isopropyl).

Cox-2 selective inhibitors such as 1H-indole derivatives described in U.S. Pat. No. 6,599,929 are useful in the present invention. Such 1H-indole derivatives have the formula shown below in formula XXXXI:

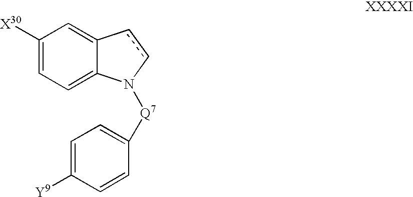

wherein:

X³⁰ is —NHSO₂R²⁰⁹ wherein R²⁰⁹ represents hydrogen or C₁-C₃-alkyl;

Y⁹ is hydrogen, halogen, C₁-C₃-alkyl substituted or not substituted by halogen, NO₂, NH₂, OH, OMe, CO₂H, or CN; and

Q⁷ is C═O, C═S, or CH₂.

Compounds that are useful as Cox-2 selective inhibitors of the present invention include prodrugs of Cox-2 inhibitors that are described in U.S. Pat. Nos. 6,436,967 and 6,613,790. Such prodrugs of Cox-2 inhibitors have the formula shown below in formula XXXXII:

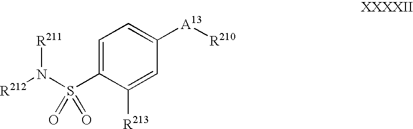

wherein:

A¹³ is a ring substituent selected from partially unsaturated heterocyclic, heteroaryl, cycloalkenyl and aryl, wherein A¹³ is unsubstituted or substituted with one or more radicals selected from alkylcarbonyl, formyl, halo, alkyl, haloalkyl, oxo, cyano, nitro, carboxyl, alkoxy, aminocarbonyl, alkoxycarbonyl, carboxyalkyl, cyanoalkyl, hydroxyalkyl, haloalkylsulfonyloxy, alkoxyalkyloxyalkyl, carboxyalkoxyalkyl, cycloalkylalkyl, alkenyl, alkynyl, heterocycloxy, alkylthio, cycloalkyl, aryl, heterocyclyl, cycloalkenyl, aralkyl, heterocyclylalkyl, alkylthioalkyl, arylcarbonyl, aralkylcarbonyl, aralkenyl, alkoxyalkyl, arylthioalkyl, aryloxyalkyl, aralkylthioalkyl, araalkoxyalkyl, alkoxycarbonylalkyl, aminocarbonylalkyl, alkylaminocarbonyl, N-arylaminocarbonyl, N-alkyl-N-arylaminocarbonyl, alkylaminocarbonylalkyl, alkylamino, -arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl;

R²¹⁰ is selected from heterocyclyl, cycloalkyl, cycloalkenyl, and aryl, wherein R²¹⁰ is unsubstituted or substituted with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy, and alkylthio;

R²¹¹ is selected from hydrido and alkoxycarbonylalkyl;

R²¹² is selected from alkyl, carboxyalkyl, acyl, alkoxycarbonyl, heteroarylcarbonyl, alkoxycarbonylalkylcarbonyl, alkoxycarbonylcarbonyl, amino acid residue, and alkylcarbonylaminoalkylcarbonyl; provided A¹³ is not tetrazolium, or pyridinium; and further provided A¹³ is not indanone when R²¹² is alkyl or carboxyalkyl; further provided A¹³ is not thienyl, when R²¹⁰ is 4-fluorophenyl, when R²¹¹ is hydrido, and when R²¹² is methyl or acyl; and

R²¹³ is hydrido;

or a pharmaceutically-acceptable salt thereof.

Specific non-limiting examples of substituted sulfonamide prodrugs of Cox-2 inhibitors disclosed in U.S. Pat. No. 6,436,967 that are useful in the present invention include:

N-[[4-[3-(difluoromethyl)-5-(3-fluoro-4-methoxyphenyl)-1H-pyrazol-1-yl]phenyl]sulfonyl]propanamide;

N-[[4-[3-(difluoromethyl)-5-(3-fluoro-4-methoxyphenyl)-1H-pyrazol-1-yl]phenyl]sulfonyl]butanamide;

N-[[4-[1,5-dimethyl)-3-phenyl-1H-pyrazol-4-yl]phenyl]sulfonyl]acetamide;

N-[[4-(2-(3-pyridinyl)-4-(trifluoromethyl)-1H-imidazol-1-yl)phenyl]sulfonyl]acetamide;

N-[[4-[2-(5-methylpyridin-3-yl)-4-(trifluoromethyl)-1H-imidazol-1-yl]phenyl]sulfonyl]acetamide;

N-[[4-[2-(2-methylpyridin-3-yl)-4-(trifluoromethyl)-1H-imidazol-1-yl]phenyl]sulfonyl]acetamide;

N-[[4-[2-(5-methylpyridin-3-yl)-4-(trifluoromethyl)-1H-imidazol-1-yl]phenyl]sulfonyl]butanamide;

N-[[4-[2-(2-methylpyridin-3-yl)-4-(trifluoromethyl)-1H-imidazol-1-yl]phenyl]sulfonyl]butanamide;

N-[[4-[2-(3-chloro-5-methylphenyl)-4-(trifluoromethyl)-1H-imidazol-1-yl]phenyl]sulfonyl]acetamide;

N-[[4-[3-(3-fluorophenyl)-5-methylisoxazol-4-yl]phenyl]sulfonyl]acetamide;

2-methyl-N-[[4-(5-methyl-3-phenylisoxazol-4-yl)phenyl]sulfonyl]propanamide;

N-[[4-(5-methyl-3-phenylisoxazol-4-yl]phenyl]sulfonyl]propanamide;

N-[[4-(5-methyl-3-phenylisoxazol-4-yl)phenyl]sulfonyl]benzamide;

2,2-dimethyl-N-[[4-(5-methyl-3-phenylisoxazol-4-yl)phenyl]sulfonyl]propanamide;

N-[[4-5-methyl-3-phenylisoxazol-4-yl)phenyl]sulfonyl]butanamide;

N-[[4-(5-methyl-3-phenylisoxazol-4-yl)phenyl]sulfonyl]pentanamide;

N-[[4-(5-methyl-3-phenylisoxazol-4-yl)phenyl]sulfonyl]hexanamide;

3-methoxy-N-[[4-(5-methyl-3-phenylisoxazol-4-yl)phenyl]sulfonyl]propanamide;

2-ethoxy-N-[[4-(5-methyl-3-phenylisoxazol-4-yl)phenyl]sulfonyl]acetamide;

N-[[4-[5-methyl-3-phenylisoxazol-4-yl]phenyl]sulfonyl]acetamide;

N-[[4-[5-(4-chlorophenyl)-3-(trifluoromethyl)-1H pyrazol-1-yl]phenyl]sulfonyl]propanamide;

N-[[4-[5-(4-chlorophenyl)-3-(trifluoromethyl)-1H-pyrazol-1-yl]phenyl]sulfonyl]butanamide;

N-[[4-[5-(4-chlorophenyl)-3-(trifluoromethyl)-1H-pyrazol-1-yl]phenyl]sulfonyl]acetamide;

N-[[4-[3-(difluoromethyl)-6-fluoro-1,5-dihydro-7-methoxy-[2]benzothiopyrano[4,3-c]pyrazol-1-yl)phenyl]sulfonyl]acetamide;

N-[[4-[6-fluoro-1,5-dihydro-7-methoxy-3-(trifluoromethyl)-[2]benzothiopyrano[4,3-c]pyrazol-1-yl]phenyl]sulfonyl]acetamide;

N-[[4-[3-(difluoromethyl)-5-(3-fluoro-4-methoxyphenyl)-1H-pyrazol-1-yl]phenyl]sulfonyl]acetamide;

N-[[4-(2-methyl-4-phenyloxazol-5-yl)phenyl]sulfonyl]acetamide;

methyl[[[4-(5-methyl-3-phenylisoxazol-4-yl)phenyl]sulfonyl]amino]oxoacetate;

2-methoxy-N-[[4-(5-methyl-3-phenylisoxazol-4-yl)phenyl]sulfonyl]acetamide;

N-[[4-[5-(difluoromethyl)-3-phenylisoxazol-4-yl]phenyl]sulfonyl]propanamide;

N-[[4-[5-(difluoromethyl)-3-phenylisoxazol-4-yl]phenyl]sulfonyl]butanamide;

N-[[4-(5-methyl-3-phenylisoxazol-4-yl)phenyl]sulfonyl]formamide;

1,1-dimethylethyl-N-[[4-(5-methyl-3-phenylisoxazol-4-yl)phenyl]sulfonyl]carbamate;

N-[[.sup.4-(5-methyl-3-phenylisoxazol-4-yl)phenyl]sulfonyl]glycine;

2-amino-N-[[4-(5-methyl-3-phenylisoxazol-4-yl)phenyl]sulfonyl]acetamide;

2-(acetylamino)-N-[[4-(5-methyl-3-phenylisoxazol-4-yl)phenyl]sulfonyl]acetamide;

methyl 4-[[[4-(5-methyl-3-phenylisoxazol-4-yl)phenyl]sulfonyl]amino]-4-oxobutanoate;

methyl N-[[4-(5-methyl-3-phenylisoxazol-4-yl)phenyl]sulfonyl]carbamate;

N-acetyl-N-[[4-(5-methyl-3-phenylisoxazol-4-yl)phenyl]sulfonyl]glycine, ethyl ester;

N-[[4-(5-(4-methylphenyl)-3-(trifluoromethyl)-1H-pyrazol-1-yl)phenyl]sulfonyl]acetamide;

methyl 3-[[[4-(5-methyl-3-phenylisoxazol-4-yl)phenyl]sulfonyl]amino]-3-oxopropanoate;

4-[5-(3-bromo-5-fluoro-4-methoxyphenyl)-2-(trifluoromethyl)oxazol-4-yl]-N-methylbenezenesulfonamide;

N-(11,1-dimethylethyl)-4-(5-methyl-3-phenylisoxazol-4-yl)benzenesulfonamide;

4-[5-(4-fluorophenyl)-3-(trifluoromethyl)-1H-pyrazol-1-yl]-N-methylbenzenesulfonamide;

N-methyl-4-(5-methyl-3-phenylisoxazol-4-yl)benezenesulfonamide;

N-[[4-[5-(hydroxymethyl)-3-phenylisoxazol-4-yl]phenyl]sulfonyl]acetamide:

N-[[4-[5-(acetoxymethyl)-3-phenylisoxazol-4-yl]phenyl]sulfonyl]acetamide;

N-[[4-[2-(3-chloro-4-fluorophenyl)cyclopenten-1-yl)phenyl]sulfonyl]acetamide;

4-[2-(4-fluorophenyl)-1H-pyrrol-1-yl]-N-methylbenzenesulfonamide;

N-[[4-(3,4-dimethyl-1-phenyl-1H-pyrazol-5-yl]phenyl]sulfonyl]propanamide;

N-[[4-[2-(2-methylpyridin-3-yl)-4-trifluoromethylimidazol-1-yl]phenyl]sulfonyl]propanamide;

4-[2-(4-fluorophenyl)cyclopenten-1-yl]-N-methylbenezenesulfonamide; and

N-[[4-(3-phenyl-2,3-dihydro-2-oxofuran-4-yl)phenyl]sulfonyl]propanamide.

Those prodrugs disclosed in U.S. Pat. No. 6,613,790 have the general formula shown above in formula XXXXII wherein:

A¹³ is a pyrazole group optionally substituted at a substitutable position with one or more radicals independently selected at each occurrence from the group consisting of alkylcarbonyl, formyl, halo, alkyl, haloalkyl, oxo, cyano, intro, carboxyl, alkoxy, aminocarbonyl, alkoxycarbonyl, carboxyalkyl, cyanoalkyl, hydroxyalkyl, haloalkylsulonyloxy, alkoxyalkyloxyalkyl, carboxyalkoxyalkyl, alkenyl, alkynyl, alkylthio, alkylthioalkyl, alkoxyalkyl, alkoxycarbonylalkyl, aminocarbonylalkyl, alkylaminocarbonyl, alkylaminocarbonylalkyl, alkylamino, aminoalkyl, alkylaminoalkyl, alkylsulfinyl, alkylsulfonyl, aminosulfonyl, and alkylaminosulfonyl;

R²¹⁰ is a phenyl group optionally substituted at a substitutable position with one or more radicals independently selected at each occurrence from the group consisting of alkyl, haloalkyl, cyano, carboxyl, alkoxycarbonyl, hydroxyl, hydroxyalkyl, haloalkoxy, amino, alkylamino, nitro, alkoxyalkyl, alkylsulfinyl, halo, alkoxy, and alkylthio;

R²¹¹ and R²¹² are independently selected from the group consisting of hydroxyalkyl and hydrido but at least one of R²¹¹ and R²¹² is other than hydrido; and

R²¹³ is selected from the group consisting of hydrido and fluoro.

Examples of prodrug compounds disclosed in U.S. Pat. No. 6,613,790 that are useful as Cox-2 inhibitors of the present invention include, but are not limited to, N-(2-hydroxyethyl)-4-[5-(4-methylphenyl)-3-(trifluoromethyl)-1H-pyrazol-1-yl]benzenesulfonamide, N,N-bis(2-hydroxyethyl)-4-[5-(4-methylphenyl)-3-(trifluoromethyl)-1H-pyrazol-1-yl]benzenesulfonamide, or pharmaceuticaly-acceptable salts thereof.

Cox-2 selective inhibitors such as sulfamoylheleroaryl pyrazole compounds that are described in U.S. Pat. No. 6,583,321 may serve as Cox-2 inhibitors of the present invention. Such sulfamoylheleroaryl pyrazole compounds have the formula shown below in formula XXXXIII:

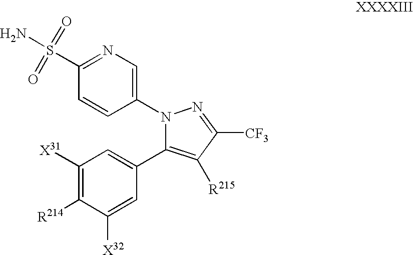

wherein:

R²¹⁴ is furyl, thiazolyl or oxazolyl;

R²¹⁵ is hydrogen, fluoro or ethyl; and

X³¹ and X³² are independently hydrogen or chloro.

Heteroaryl substituted amidinyl and imidazolyl compounds such as those described in U.S. Pat. No. 6,555,563 are useful as Cox-2 selective inhibitors of the present invention. Such heteroaryl substituted amidinyl and imidazolyl compounds have the formula shown below in formula XXXXIV:

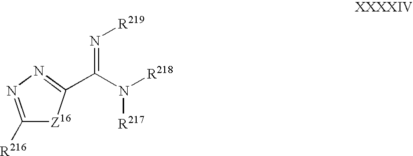

wherein:

Z¹⁶ is O or S,

R²¹⁶ is optionally substituted aryl,

R²¹⁷ is aryl optionally substituted with aminosulfonyl, and

R²¹⁸ and R²¹⁹ cooperate to form an optionally substituted 5-membered ring.

Materials that can serve as Cox-2 selective inhibitors of the present invention include substituted hydroxamic acid derivatives that are described in U.S. Pat. Nos. 6,432,999, 6,512,121, and 6,515,014. These compounds also act as inhibitors of the lipoxygenase-5 enzyme. Such substituted hydroxamic acid derivatives have the general formulas shown below in formulas XXXXV and XXXXVI:

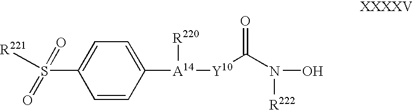

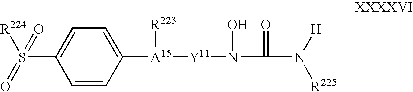

Pyrazole substituted hydroxamic acid derivatives described in U.S. Pat. No. 6,432,999 have the formula shown above in formula XXXXV, wherein:

A¹⁴ is pyrazolyl optionally substituted with a substituent selected from acyl, halo, hydroxyl, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl;

Y¹⁰ is selected from lower alkenylene and lower alkynylene;

R²²⁰ is a substituent selected from 5- and 6-membered heterocyclo, lower cycloalkyl, lower cycloalkenyl and aryl selected from phenyl, biphenyl and naphthyl, wherein R²²⁰ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylmino, nitro, lower alkoxyalkyl, lower alkylsulfinyl, halo, lower alkoxy and lower alkylthio;

R²²¹ is selected from lower alkyl and amino; and

R²²² is selected from hydrido, lower alkyl, phenyl, 5- and 6-membered heterocyclo and lower cycloalkyl; or a pharmaceutically-acceptable salt thereof.

Pyrazole substituted hydroxamic acid derivatives described in U.S. Pat. No. 6,432,999 may also have the formula shown above in formula XXXXVI, wherein:

A¹⁵ is pyrazolyl optionally substituted with a substituent selected from acyl, halo, hydroxyl, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl;

Y¹¹ is selected from lower alkylene, lower alkenylene and lower alkynylene;

R²²³ is a substituent selected from 5- and 6-membered heterocyclo, lower cycloalkyl, lower cycloalkenyl and aryl selected from phenyl, biphenyl and naphthyl, wherein R²²³ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylmino, nitro, lower alkoxyalkyl, lower alkylsulfinyl, halo, lower alkoxy and lower alkylthio;

R²²⁴ is selected from lower alkyl and amino; and

R²²⁵ is selected from hydrido, lower alkyl;

or a pharmaceutically-acceptable salt thereof.

Heterocyclo substituted hydroxamic acid derivatives described in U.S. Pat. No. 6,512,121 have the formula shown above in formula XXXXV, wherein:

A¹⁴ is a ring substiuent selected from oxazolyl, furyl, pyrrolyl, thiazolyl, imidazolyl, isochiazolyl, isoxazolyl, cyclopentenyl, phenyl, and pyridyl; wherein A¹⁴ is optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl;

Y¹⁰ is lower alkylene, lower alkenylene, and lower alkynylene;

R²²⁰ is a substituent selected from 5- and 6-membered heterocyclo, lower cycloalkyl, lower cycloalkenyl and aryl selected from phenyl, biphenyl and naphthyl, wherein R²²⁰ is otionallv substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;

R²²¹ is selected from lower alkyl and amino; and

R²²² is selected from hydrido, lower alkyl, phenyl, 5- and 6-membered heterocyclo and lower cycloalkyl; or a pharmaceutically-acceptable salt thereof.

Heterocyclo substituted hydroxamic acid derivatives described in U.S. Pat. No. 6,512,121 may also have the formula shown above in formula XXXXVI, wherein:

A¹⁵ is a ring substituent selected from oxazolyl, furyl, pyrrolyl, thiazolyl, imidazolyl, isothiazolyl, isoxazolyl, cyclopentenyl, phenyl, and pyridyl; wherein A is optionally substituted with a substituent selected from acyl, halo, hydroxy, lower alkyl, lower haloalkyl, oxo, cyano, nitro, carboxyl, lower alkoxy, aminocarbonyl, lower alkoxycarboryl, lower carboxyalkyl, lower cyanoalkyl, and lower hydroxyalkyl;

Y¹¹ is selected from lower alkyl, lower alkenyl and lower alkynyl;

R²²³ is a substituent selected from 5- and 6-membered heterocyclo, lower cycloalkyl, lower cycloalkenyl and aryl selected from phenyl, biphenyl and naphthyl, wherein R²²³ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitto, lower alkoxyalkyl, lower alkylsulfinyl, halo, lower alkoxy and lower alkylthio;

R²²⁴ is selected from lower alkyl and amino; and

R²²⁵ is selected from hydrido and alkyl; or a pharmaceutically-acceptable salt thereof.

Thiophene substituted hydroxamic acid derivatives described in U.S. Pat. No. 6,515,014 have the formula shown above in formula XXXXV, wherein:

A¹⁴ is thienyl optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl;

Y¹⁰ is ethylene, isopropylene, propylene, butylene, lower alkenylene, and lower alkynylene;

R²²⁰ is a substituent selected from 5- and 6-membered heterocyclo, lower cycloalkyl, lower cycloalkenyl and aryl selected from phenyl, biphenyl and naphthyl, wherein R²²⁰ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;

R²²¹ is selected from lower alkyl and amino; and

R²²² is selected from hydrido, lower alkyl, phenyl, 5- and 6-membered heterocyclo and lower cycloalkyl; or a pharmaceutically-acceptable salt thereof.

Thiophene substituted hydroxamic acid derivatives described in U.S. Pat. No. 6,515,014 may also have the formula shown above in formula XXXXV, wherein:

A¹⁵ is thienyl optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl;

Y¹¹ is selected from lower alkyl, lower alkenyl and lower alkynyl;

R²²³ is a substituent selected from 5- and 6-membered heterocyclo, lower cycloalkyl, lower cycloalkenyl and aryl selected from phenyl, biphenyl and naphthyl, wherein R²²³ is optionally substituted at a substitutable position with one or more substituents selected from lower alkyl, lower haloalkyl, cyano, carboxyl, lower alkoxycarbonyl, hydroxyl, lower hydroxyalkyl, lower haloalkoxy, amino, lower alkylamino, phenylamino, nitro, lower alkoxyalkyl, lower alkylsulfinyl, halo, lower alkoxy and lower alkylthio;

R²²⁴ is selected from lower alkyl and amino; and

R²²⁵ is selected from hydrido and alkyl; or a pharmaceutically-acceptable salt thereof.

Compounds that are useful as Cox-2 selective inhibitors of the present invention include pyrazolopyridine compounds that are described in U.S. Pat. No. 6,498,166. Such pyrazolopyridine compounds have the formula shown below in formula XXXXVII:

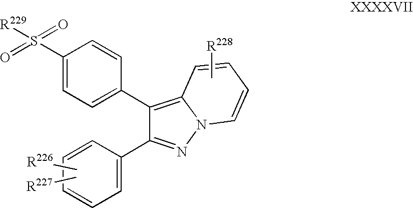

wherein:

R²²⁶ and R²²⁷ are independently selected from the group consisting of H, halogen, C₁-C₆ alkyl, C₁-C₆ alkoxy, and C₁-C₆ alkoxy substituted by one or more fluorine atoms;

R²²⁸ is halogen, CN, CONR²³⁰R²³¹, CO₂H, CO₂C₁-C₆ alkyl or NHSO₂R²³⁰;

R²²⁹ is C₁-C₆ alkyl or NH₂; and

R²²⁵ and R²²⁵ are independently selected from the group consisting of H, C₁-C₆ alkyl, phenyl, phenyl substituted by one or more atoms or groups selected from the group consisting of halogen, C₁-C₆ alkyl, C₁-C₆ alkoxy, and C₁-C₆ alkoxy substituted by one or more fluorine atoms,

or a pharmaceutically acceptable salt, solvate, ester, or salt or solvate of such ester thereof.

Materials that are useful as Cox-2 selective inhibitors of the present invention include 4,5-diaryl-3(2H)-furanone derivatives that are described in U.S. Pat. No. 6,492,416. Such 4,5-diaryl-3(2H)-furanone derivatives have the formula shown below in formula XXXXVIII:

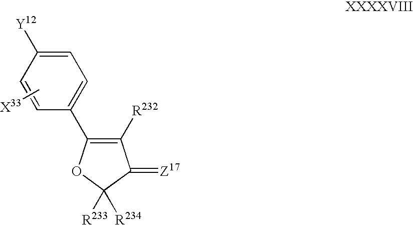

wherein:

X³³ represents halo, hydrido, or alkyl;

Y¹² represents alkylsulfonyl, aminosulfonyl, alkylsulfinyl, (N-acylamino)-sulfonyl, (N-alkylamino)sulfonyl, or alkylthio;

Z¹⁷ represents oxygen or sulfur atom; R²²³ and R²³⁴ are selected independently from lower alkyl radicals; and R²³² represents a substituted or non-substituted aromatic group of 5 to 10 atoms;

or a pharmaceutically-acceptable salt thereof.

Cox-2 selective inhibitors that can be used in the present invention include 2-phenyl-1,2-benzisoselenazol-3(2H)-one derivatives and 2-phenylcarbomyl-phenylselenyl derivatives that are described in U.S. Pat. No. 6,492,416. Such 2-phenyl-1,2-benzisoselenazol-3(2H)-one derivatives and 2-phenylcarbomyl-phenylselenyl derivatives have the formulas shown below in formulas XXXXIX or XXXXIX′:

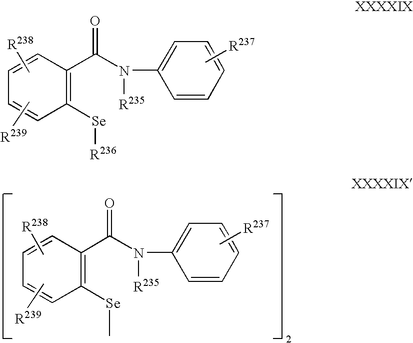

wherein:

R²³⁵ is a hydrogen atom or an alkyl group having 1-3 carbon atoms;

R²³⁶ is a hydrogen atom, a hydroxyl group, an organothiol group that is bound to the selenium atom by its sulfur atom, or R²³⁵ and R²³⁶ are joined to each other by a single bond;

R²³⁷ is a hydrogen atom, a halogen atom, an alkyl group having 1-3 carbon atoms, an alkoxyl group having 1-3 carbon atoms, a trifluoromethyl group, or a nitro group;

R²³⁸ and R²³⁹ are identical to or different from each other, and each is a hydrogen atom, a halogen atom, an alkoxyl group having 1-4 carbon atoms, a trifluoromethyl group, or R²³⁸ and R²³⁹ are joined to each other to form a methylenedioxy group,

a salt thereof, or a hydrate thereof.

Pyrones such as those disclosed in U.S. Pat. No. 6,465,509 are also useful as Cox-2 inhibitors of the present invention. These pyrone compounds have the general formula shown below in formula XXXXX:

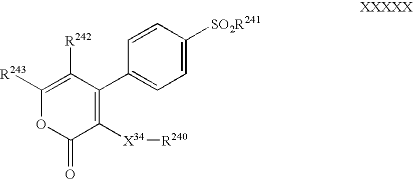

wherein:

X³⁴ is selected from the group consisting of:

(a) a bond,

(b) —(CH₂)ₘ—, wherein m 1 or 2,

(c) —C(O)—,

(d) —O—,

(e) —S—, and

(f) —N(R²⁴⁴)—;

R²⁴⁰ is selected from the group consisting of:

(a) C₁-C₁₀ alkyl, optionally substituted with 1-3 substituents independently selected from the group consisting of: hydroxy, halo, C₁-C₁₀ alkoxy, C₁-C₁₀ alkylthio, and CN,

(b) phenyl or naphthyl, and

(c) heteroaryl, which is comprised of a monocyclic aromatic ring of 5 atoms having one hetero atom which is S, O or N, and optionally 1, 2, or 3 additional N atoms; or

a monocyclic ring of 6 atoms having one hetero atom which is N, and optionally 1, 2, or 3 additional N atoms, wherein groups (b) and (c) above are each optionally substituted with 1-3 substituents independently selected from the group consisting of: halo, C₁-C₁₀ alkoxy, C₁-C₁₀ alkylthio, CN, C₁-C₁₀ alkyl, optionally substituted to its maximum with halo, and N₃;

R²⁴¹ is selected from the group consisting of

(a) C₁-C₆ alkyl, optionally substituted to its maximum with halo,

(b) NH₂, and

(c) NHC(O)C₁-C₁₀ alkyl, optionally substituted to its maximum with halo;

R²⁴² and R²⁴³ are each independently selected from the group consisting of: hydrogen, halo, and C₁-C₆ alkyl, optionally substituted to its maximum with halo; and

R²⁴⁴ is selected from the group consisting of: hydrogen and C₁-C₆ alkyl, optionally substituted to its maximum with halo.

Examples of pyrone compounds that are useful as Cox-2 selective inhibitors of the present invention include, but are not limited to:

4-(4-Methylsulfonyl)phenyl-3-phenyl-pyran-2-one,

3-(4-Fluorophenyl)-6-methyl-4-(4-methylsulfonyl)phenyl-pyran-2-one,

3-(3-Fluorophenyl)-6-methyl-4-(4-methylsulfonyl)phenyl-pyran-2-one,

6-Methyl-4-(4-methylsulfonyl)phenyl-3-phenyl-pyran-2-one,

6-Difluoromethyl-4-(4-methylsulfonyl)phenyl-3-phenyl-pyran-2-one,

6-Fluoromethyl-4-(4-methylsulfonyl)phenyl-3-phenyl-pyran-2-one,

6-Methyl-4-(4-methylsulfonyl)phenyl-3-phenylthio-pyran-2-one,

6-Methyl-4-(4-methylsulfonyl)phenyl-3-phenoxy-pyran-2-one,

6-Methyl-4-(4-methylsulfonyl)phenyl-3-pyridin-3-yl-pyran-2-one,

3-Isopropylthio-6-methyl-4-(4-methylsulfonyl)phenyl-pyran-2-one,

4-(4-Methylsulfonyl)phenyl)-3-phenylthio-6-trifluoromethyl-pyran-2-one,

3-Isopropylthio-4-(4-methylsulfonyl)phenyl-6-trifluoromethyl-pyran-2-one,

4-(4-Methylsulfonyl)phenyl-3-phenyl-6-(2,2,2-trifluoroethyl)-pyran-2-one, and

3-(3-Hydroxy-3-methylbutyl)-6-methyl-4-(4-methylsulfonyl)phenyl-pyran-2-one.

Organically synthesized or purified from plant sources, free-B-ring flavanoids such as those described in U.S. Published Application No. 2003/0165588, are useful as Cox-2 selective inhibitors of the present invention. Such free-B-ring flavanoids have the general structure shown in formula XXXXXI:

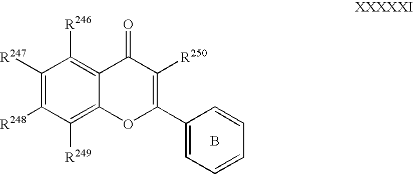

wherein:

R²⁴⁶, R²⁴⁷, R²⁴⁸, R²⁴⁹, and R²⁵⁰ are independently selected from the group consisting of: —H, —OH, —SH, —OR, —SR, —NH₂, —NHR²⁴⁵, —N(R²⁴⁵)₂, —N(R²⁴⁵)₃⁺X³⁵⁻, a carbon, oxygen, nitrogen or sulfur, glycoside of a single or a combination of multiple sugars including, aldopentoses, methyl-aldopentose, aldohexoses, ketohexose and their chemical derivatives thereof; wherein R²⁴⁵ is an alkyl group having between 1-10 carbon atoms; and X³⁵ is selected from the group of pharmaceutically acceptable counter anions including, hydroxyl, chloride, iodide, sulfate, phosphate, acetate, fluoride and carbonate.

Heterocyclo-alkylsulfonyl pyrazoles such as those described in European Patent Application No. EP 1312367 are useful as Cox-2 selective inhibitors of the present invention. Such heterocyclo-alkylsulfonyl pyrazoles have the general formula shown below in formula XXXXXII:

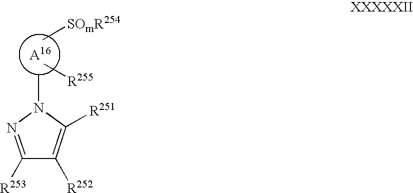

or a pharmaceutically acceptable salt thereof, wherein: the ring of the formula (R²⁵⁵)-A-(SOₘR²⁵⁴) is selected from the group consisting of:

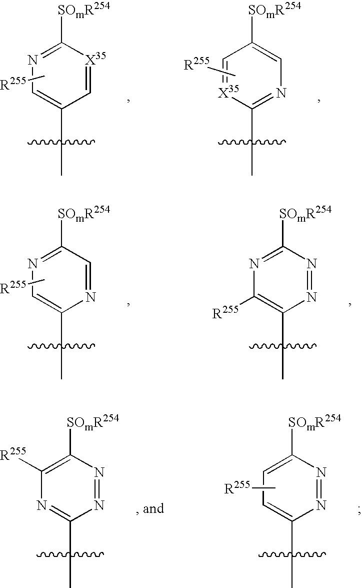

m is 0, 1 or 2;

X³⁵ is >CR²⁵⁵ or >N; R²⁵¹ is a radical selected from the group consisting of H, NO₂, CN, (C₁-C₆)alkyl, (C₁-C₆)alkyl-SO₂—, (C₆-C₁₀)aryl-SO₂—, H—(C═O)—, (C₁-C₆)alkyl-(C═O)—, (C₁-C₆)alkyl-)-(C═O)—, (C₁-C₉)heteroaryl-(C═O)—, (C₁-C₉)heterocyclyl-(C═O)—, H₂N—(C═O)—, (C₁-C₆)alkyl-NH—(C═O)—, [(C₁-C₆)alkyl]₂—N—(C═O)—, [(C₆-C₁₀)aryl]₂—NH—(C═O)—, [(C₁-C₆)alkyl]-[((C₆-C₁₀)aryl-N]-(C═O)—, HO—NH—(C═O)—, and (C₁-C₆)alkyl-O—NH—(C═O)—;

R²⁵² is a radical selected from the group consisting of H, —NO₂, —CN, (C₂-C₆)alkenyl, (C₂-C₆)alkynyl, (C₃-C₇)cycloalkyl, (C₆-C₁₀)aryl, (C₁-C₉)heteroaryl, (C₁-C₉)heterocyclyl, (C₁-C₆)alkyl-O—, (C₃-C₇)cycloalkyl-O—, (C₆-C₁₀)aryl-O—, (C₁-C₉)heteroaryl-O—, (C₆-C₉)heterocyclyl-O—, H—(C═O)—, (C₁-C₆)alkyl-(C═O)—, (C₃-C₇)cycloalkyl-(C═O)—, (C₆-C₁₀)aryl-(C═O)—, (C₁-C₉)heteroaryl-(C═O)—, (C₁-C₉)heterocyclyl-(C═O)—, (C₁-C₆)alkyl-O—(C═O)—, (C₃-C₇)cycloalkyl-O—(C═O)—, (C₆-C₁₀)aryl-O—(C═O)—, (C₁-C₉)heteroaryl-O—(C═O)—, (C₁-C₉)heterocyclyl-O—(C═O)—, (C₁-C₆)alkyl-(C═O)—O—, (C₃-C₇)cycloalkyl-(C═O)—O—, (C₆-C₁₀)aryl-(C═O)—O—, (C₁-C₉)heteroaryl-(C═O)—O—, (C₁-C₉)heterocyclyl-(C═O)—O—, (C₁-C₆)alkyl-(C═O)—NH—, (C₃-C₇)cycloalkyl-(C═O)—NH—, (C₆-C₁₀aryl-(C═O)—NH—. (C₁-C₉)heteroaryl-(C═O)—NH—, (C₁-C₉)heterocyclyl-(C═O)—NH—, (C₁-C₆)alkyl-O—(C═O)—NH—, (C₁-C₆)alkyl-NH, [(C₁-C₆)alkyl]₂—N—, (C₃-C₇)cycloalkyl-NH—. [(C₃-C₇)cycloalkyl]₂—N—, [(C₆-C₁₀)aryl]-NH—, [(C₆-C₁₀)aryl]₂—N—, [(C₁-C₆)alkyl]-[((C₆-C₁₀)aryl)-N]—, [(C₁-C₉)heteroaryl]-NH—, [(C₁-C₉)heteroaryl]₂—N—, [(C₁-C₉)heterocycly]-NH—, [(C₁-C₉)heterocyclyl]₂—N—, H₂N—(C═O)—, HO—NH—(C═O)—, (C₁-C₆)alkyl-O—NH—(C═O)—, [(C₁-C₆)alkyl]-NH—(C═O)—, [(C₁-C₆)alkyl]₂—N—(C═O)—, [(C₃-C₇)cycloalkyl]-NH—(C═O)—, [(C₃-C₇)cycloalkyl]₂—N—(C═O)—, [(C₆-C₁₀)aryl]-NH—(C═O)—, [(C₆-C₁₀aryl]₂—N—(C═O)—, [(C₁-C₆)alkyl]-[((C₆-C₁₀)aryl)-N]-(C═O)—, [(C₁-C₉)heteroaryl]-NH—(C═O)—, [(C₁-C₉)heteroaryl]₂—N—(O═O)—, [(C₁-C₉)heterocyclyl]-NH—(C═O)—, (C₁-C₆)alkyl-S— and (C₁-C₆)alkyl optionally substituted by one —OH substituent or by one to four fluoro substituents;

R²⁵³ is a saturated (3- to 4-membered)-heterocyclyl ring radical; or a saturated, partially saturated or aromatic (7- to 9-membered)-heterocyclyl ring radical;

wherein said saturated (3- to 4-membered)-heterocyclyl ring radical or said saturated, partially saturated or aromatic (7- to 9-membered)-heterocyclyl ring radical; may optionally contain one to four ring heteroatoms independently selected Irom the groups consisting of —N═, —NH—, —O—, and —S—;

wherein said saturated (3- to 4-membered)-heterooyclyl ring radical; or said saturated, partially saturated or aromatic (7- to 9-nembered)-heterocyclyl ring radical; may optionally be substituted on any ring carbon atom by one to three substituents per ring independently selected from the group consisting of halo, —OH, —CN, —NO₂, (C₂-C₆)alkenyl, (C₂-C₆)alkynyl, (C₃-C₇)cycloalkyl, (C₆-C₁₀)aryl, (C₂-C₉)hetorocyclyl, (C₁-C₆)alkyl-O—, H—(C═O)—, (C₁-C₆)alkyl-(C═O)—, HO—(C═O)—, (C₁-C₆)alkyl-O—(C═O)—, —NH₂, (C₁-C₆)alkyl-NH—, [(C₁-C₆)alkyl]₂—N—, (C₃-C₇)cycloalkyl-NH—, (C₆-C₁₀)aryl-NH—, [(C₁-C₆)alkyl]-[((C₆-C₁₀)aryl)-N]-, (C₁-C₉)heteroaryl-NH—, H₂N—(C═O)-[(C₁-C₆)alkyl]-NH—(C═O)—, [(C₁-C₆)alkyl]₂—N—(C═O)—, [(C₆-C₁₀)aryl]-NH—(C═O)—, [(C₁-C₆)alkyl]-[((C₆-C₁₀)aryl)-N]-(C═O)—, (C₁-C₆)alkyl-O—NH—(C═O)—, (C₁-C₆)alkyl-(C═O)—HN—, (C₁-C₆)alkyl-(C═O)-[(C₁-C₆)alkyl-N]—, —SH, (C₁-C₆)alkyl-S—, (C₁-C₆)alkyl-(S═O)-, (C₁-C₆)alkyl-SO₂— and (C₁-C₆)alkyl optionally substituted with one to fourfluoro moieties;

wherein said saturated (3- to 4-membered)-heterocyclyl ring radical; or said saturated, partially saturated or aromatic (7- to 9-membered)-heterocyclyl ring radical; may also optionally be substituted on any ring nitrogen atom by one to three substituents per ring independently selected from the group consisting of (C₃-C₇)cyoloalkyl, (C₆-C₁₀)aryl, (C₂-C₉)heterocyclyl, H—(C═O)—, (C₁-C₆)alkyl-(C═O)—, (C₁-C₆)alkyl-O—(C═O)—, H₂N—(C═O)—, [(C₁-C₆)alkyl]-NH—(C═O)—, [(C₁-C₆)alkyl]₂—N—(C═O)—, [(C₆-C₁₀)aryl]-NH—(C═O)—, [(C₁-C₆)alkyl]-[((C₆-C₁₀)aryl)-N]-(C═O)—, (C₁-C₆)alkyl-O—NH—(C═O)—, and (C₁-C₆)alkyl optionally substituted with one to four fluoro moieties;

R²⁵⁴ is an (C₁-C₆)alkyl radical optionally substituted by one to four fluoro substituents; and

R²⁵⁵ is a radical selected from the group consisting of H, halo, —OH, (C₁-C₆)alkyl-O—, (C₂-C₆)alkenyl, (C₂-C₆)alkynyl, (C₃-C₇)cycloalkyl, —CN, H—(C═O)—, (C₁-C₆)alkyl-(C═O)—, (C₁-C₆)alkyl-(C═O)—O—, HO—(C═O)—, (C₁-C₆)alkyl-O—(C═O)—, (C₁-C₆)alkyl-NH—. [(C₁-C₆)alkyl]₂—N—, (C₃-C₇)cycloalkyl-NH—, (C₆-C₁₀)aryl-NH—, [(C₁-C₆)alkyl]-[((C₆-C₁₀)aryl)-N]—, (C₁-C₉)heteroaryl-NH—, H₂N—(C═O)—, (C₁-C₆)alkyl-NH—(C═O)—. [(C₁-C₆)alkyl]₂—N—(C═O)—, (C₆-C₁₀)aryl-(C═O)—, [(C₁-C₆)alkyl]-[((C₆-C₁₀)aryl)-N]-(C═O)—, (C₁-C₆)alkyl-O—NH—(C═O)—, (C₁-C₆)alkyl-S—, and (C₁-C₆)alkyl optionally substituted by one to four fluoro substituents.

2-phenylpyran-4-one derivatives such as those described in U.S. Pat. No. 6,518,303 are also useful as Cox-2 selective inhibitors of the present invention. Such 2-phenylpyran-4-one derivatives have the general formula shown below in formula XXXXXIII:

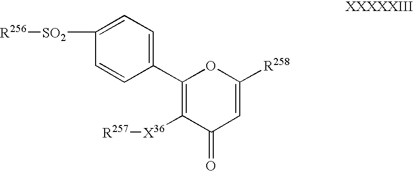

wherein:

R²⁵⁶ represents an alkyl or —NR²⁵⁹R²⁶⁰ group, wherein R²⁵⁹ and R²⁶⁰ each independently represents a hydrogen atom or an alkyl group;

R²⁵⁷ represents an alkyl, C₃-C₇ cycloalkyl, naphthyl, tetrahydronaphthyl or indanyl group, or a phenyl group which may be unsubstituted or substituted by one or more halogen atoms or alkyl, trifluoromethyl, hydroxy, alkoxy, methylthio, amino, mono- or dialkylamino, hydroxyalkyl or hydroxycarbonyl groups;

R²⁵⁸ represents a methyl, hydroxymethyl, alkoxymethyl, C₃-C₇ cycloalkoxymethyl, benzyloxymethyl, hydroxycarbonyl, nitrile, trifluoromethyl or difluoromethyl group or a CH₂—R²⁶¹ group wherein R²⁶¹ represents an alkyl group; and

X³⁶ represents a single bond, an oxygen atom, a sulfur atom or a methylene group;

or a pharmaceutically acceptable salt thereof.

Examples of 2-phenylpyran-4-one derivatives useful in the present invention include, but are not limited to:

3-(4-fluorophenyl)-2-(4-methanesulfonylphenyl)-6-methylpyran-4-one,

3-(2-fluorophenyl)-2-(4-methanesulfonylphenyl)-6-methylpyran-4-one,

3-(4-chlorophenyl)-2-(4-methanesulfonylphenyl)-6-methylpyran-4-one,

3-(4-bromophenyl)-2-(4-methylsulfonylphenyl)-6-methylpyran-4-one,

3-(2,4-difluorophenyl)-2-(4-methanesulfonylphenyl)-6-methylpyran-4-one,

3-(3,4-dichlorophenyl)-2-(4-methanesulfonylphenyl)-6-methylpyran-4-one,

3-(3-chloro-4-methylphenyl)-2-(4-methanesulfonylphenyl)-6-methylpyran-4-one,

2-(4-methanesulfonylphenyl)-6-methyl-3-phenoxypyran-4-one,

3-(4-fluorophenoxy)-2-(4-methanesulfonylphenyl)-6-methylpyran-4-one,

3-(2-fluorophenoxy)-2-(methanesulfonylphenyl)-6-methylpyran-4-one,

3-(4-chlorophenoxy)-2-(methanesulfonylphenyl)-6-methylpyran-4-one,

3-(2-chlorophenoxy)-2-(methanesulfonylphenyl)-6-methylpyran-4-one,

3-(4-bromophenoxy)-2-(4-methanesulfonylphenyl)-6-methylpyran-4-one,

2-(4-methanesulfonylphenyl)-6-methyl-3-(4-methylphenoxy)pyran-4-one,

3-(2,4-difluorophenoxy)-2-(4-methanesulfonylphenyl)-6-methylpyran-4-one,

3-(2,5-difluorophenoxy)-2-(methanesulfonylphenyl)-6-methylpyran-4-one,

3-(4-chlorophenyl)-2-(4-methanesulfonylphenyl)-6-methoxymethylpyran-4-one,

3-(4-chlorophenyl)-6-difluoromethyl-2-(4-methanesulfonylphenyl)pyran-4-one,

and pharmaceutically acceptable salts thereof.

Cox-2 selective inhibitors that are useful in the subject method and compositions can include the compounds that are described in U.S. Pat. No. 6,472,416 (sulfonylphenylpyrazoles); U.S. Pat. No. 6,451,794 (2,3-diaryl-pyrazolo[1,5-b]pyridazines); U.S. Pat. Nos. 6,169,188, 6,020,343, and 5,981,576 ((methylsulfonyl)phenyl furanones); U.S. Pat. No. 6,222,048 (diaryl-2-(5H)-furanones); U.S. Pat. No. 6,057,319 (3,4-diaryl-2-hydroxy-2,5-dihydrofurans); U.S. Pat. No. 6,046,236 (carbocyclic sulfonamides); U.S. Pat. Nos. 6,002,014 and 5,945,539 (oxazole derivatives); and U.S. Pat. Nos. 6,359,182 and 6,538,116 (C-nitroso compounds).

Examples of specific compounds that are useful as Cox-2 selective inhibitors include, without limitation:

a1) 8-acetyl-3-(4-fluorophenyl)-2-(4-methylsulfonyl)phenyl-imidazo(1,2-a)pyridine;

a2) 5,5-dimethyl-4-(4-methylsulfonyl)phenyl-3-phenyl-2-(5H)-furanone;

a3) 5-(4-fluorophenyl)-1-[4-(methylsulfonyl)phenyl]-3-(trifluoromethyl)pyrazole;

a4) 4-(4-fluorophenyl)-5-[4-(methylsulfonyl)phenyl]-1-phenyl-3-(trifluoromethyl)pyrazole;

a5) 4-(5-(4-chlorophenyl)-3-(4-methoxyphenyl)-1H-pyrazol-1-yl)benzenesulfonamide

a6) 4-(3,5-bis(4-methylphenyl)-1H-pyrazol-1-yl)benzenesulfonamide;

a7) 4-(5-(4-chlorophenyl)-3-phenyl-1H-pyrazol-1-yl)benzenesulfonamide;

a8) 4-(3,5-bis(4-methoxyphenyl)-1H-pyrazol-1-yl)benzenesulfonamide;

a9) 4-(5-(4-chlorophenyl)-3-(4-methylphenyl)-1H-pyrazol-1-yl)benzenesulfonamide;

a10) 4-(5-(4-chlorophenyl)-3-(4-nitrophenyl)-1H-pyrazol-1-yl)benzenesulfonamide;

b1) 4-(5-(4-chlorophenyl)-3-(5-chloro-2-thienyl)-1H-pyrazol-1-yl)benzenesulfonamide;

b2) 4-(4-chloro-3,5-diphenyl-1H-pyrazol-1-yl)benzenesulfonamide

b3) 4-[5-(4-chlorophenyl)-3-(trifluoromethyl)-1H-pyrazol-1-yl]benzenesulfonamide;

b4) 4-[5-phenyl-3-(trifluoromethyl)-1H-pyrazol-1-yl]benzenesulfonamide;

b5) 4-[5-(4-fluorophenyl)-3-(trifluoromethyl)-1H-pyrazol-1-yl]benzenesulfonamide;

b6) 4-[5-(4-methoxyphenyl)-3-(trifluoromethyl)-1H-pyrazol-1-yl]benzenesulfonamide;

b7) 4-[5-(4-chlorophenyl)-3-(difluoromethyl)-1H-pyrazol-1-yl]benzenesulfonamide;

b8) 4-[5-(4-methylphenyl)-3-(trifluoromethyl)-1H-pyrazol-1-yl]benzenesulfonamide;

b9) 4-[4-chloro-5-(4-chlorophenyl)-3-(trifluoromethyl)-1H-pyrazol-1-yl]benzenesulfonamide;

b10) 4-[3-(difluoromethyl)-5-(4-methylphenyl)-1H-pyrazol-1-yl]benzenesulfonamide;

c1) 4-[3-(difluoromethyl)-5-phenyl-1H-pyrazol-1-yl]benzenesulfonamide;

c2) 4-[3-(difluoromethyl)-5-(4-methoxyphenyl)-1H-pyrazol-1-yl]benzenesulfonamide;

c3) 4-[3-cyano-5-(4-fluorophenyl)-1H-pyrazol-1-yl]benzenesulfonamide;

c4) 4-[3-(difluoromethyl)-5-(3-fluoro-4-methoxyphenyl)-1H-pyrazol-1-yl]benzenesulfonamide;

c5) 4-[5-(3-fluoro-4-methoxyphenyl)-3-(trifluoromethyl)-1H-pyrazol-1-yl]benzenesulfonamide;

c6) 4-[4-chloro-5-phenyl-1H-pyrazol-1-yl]benzenesulfonamide;

c7) 4-[5-(4-chlorophenyl)-3-(hydroxymethyl)-1H-pyrazol-1-yl]benzenesulfonamide;

c8) 4-[5-(4-(N,N-dimethylamino)phenyl)-3-(trifluoromethyl)-1H-pyrazol-1-yl]benzenesulfonamide;

c9) 5-(4-fluorophenyl)-6-[4-(methylsulfonyl)phenyl]spiro[2.4]hept-5-ene;

c10) 4-[6-(4-fluorophenyl)spiro[2.4]hept-5-en-5-yl]benzenesulfonamide;

d1) 6-(4-fluorophenyl)-7-[4-(methylsulfonyl)phenyl]spiro[3.4]oct-6-ene;

d2) 5-(3-chloro-4-methoxyphenyl)-6-[4-(methylsulfonyl)phenyl]spiro[2.4]hept-5-ene;

d3) 4-[6-(3-chloro-4-methoxyphenyl)spiro[2.4]hept-5-en-5-yl]benzenesulfonamide;

d4) 5-(3,5-dichloro-4-methoxyphenyl)-6-[4-(methylsulfonyl)phenyl]spiro[2.4]hept-5-ene;

d5) 5-(3-chloro-4-fluorophenyl)-6-[4-(methylsulfonyl)phenyl]spiro[2.4]hept-5-ene;

d6) 4-[6-(3,4-dichlorophenyl)spiro[2.4]hept-5-en-5-yl]benzenesulfonamide;

d7) 2-(3-chloro-4-fluorophenyl)-4-(4-fluorophenyl)-5-(4-methylsulfonylphenyl)thiazole;

d8) 2-(2-chlorophenyl)-4-(4-fluorophenyl)-5-(4-methylsulfonylphenyl)thiazole;

d9) 5-(4-fluorophenyl)-4-(4-methylsulfonylphenyl)-2-methylthiazole;

d10) 4-(4-fluorophenyl)-5-(4-methylsulfonylphenyl)-2-trifluoromethylthiazole;

e1) 4-(4-fluorophenyl)-5-(4-methylsulfonylphenyl)-2-(2-thienyl)thiazole;

e2) 4-(4-fluorophenyl)-5-(4-methylsulfonylphenyl)-2-benzylaminothiazole;

e3) 4-(4-fluorophenyl)-5-(4-methylsulfonylphenyl)-2-(1-propylamino)thiazole;

e4) 2-[(3,5-dichlorophenoxy)methyl)-4-(4-fluorophenyl)-5-[4-(methylsulfonyl)phenyl]thiazole;

e5) 5-(4-fluorophenyl)-4-(4-methylsulfonylphenyl)-2-trifluoromethylthiazole;

e6) 1-methylsulfonyl-4-[1,1-dimethyl-4-(4-fluorophenyl)cyclopenta-2,4-dien-3-yl]benzene;

e7) 4-[4-(4-fluorophenyl)-1,1-dimethylcyclopenta-2,4-dien-3-yl]benzenesulfonamide;

e8) 5-(4-fluorophenyl)-6-[4-(methylsulfonyl)phenyl]spiro[2.4]hepta-4,6-diene;

e9) 4-[6-(4-fluorophenyl)spiro[2.4]hepta-4,6-dien-5-yl]benzenesulfonamide;

e10) 6-(4-fluorophenyl)-2-methoxy-5-[4-(methylsulfonyl)phenyl]-pyridine-3-carbonitrile;

f1) 2-bromo-6-(4-fluorophenyl)-5-[4-(methylsulfonyl)phenyl]-pyridine-3-carbonitrile;

f2) 6-(4-fluorophenyl)-5-[4-(methylsulfonyl)phenyl]-2-phenyl-pyridine-3-carbonitrile;

f3) 4-[2-(4-methylpyridin-2-yl)-4-(trifluoromethyl)-1H-imidazol-1-yl]benzenesulfonamide;

f4) 4-[2-(5-methylpyridin-3-yl)-4-(trifluoromethyl)-1H-imidazol-1-yl]benzenesulfonamide;

f5) 4-[2-(2-methylpyridin-3-yl)-4-(trifluoromethyl)-1H-imidazol-1-yl]benzenesulfonamide;

f6) 3-[1-[4-(methylsulfonyl)phenyl]-4-(trifluoromethyl)-1H-imidazol-2-yl]pyridine;

f7) 2-[1-[4-(methylsulfonyl)phenyl-4-(trifluoromethyl)-1H-imidazol-2-yl]pyridine;

f8) 2-methyl-4-[1-[4-(methylsulfonyl)phenyl-4-(trifluoromethyl)-1H-imidazol-2-yl]pyridine;

f9) 2-methyl-6-[1-[4-(methylsulfonyl)phenyl-4-(trifluoromethyl)-1H-imidazol-2-yl]pyridine;

f10) 4-[2-(6-methylpyridin-3-yl)-4-(trifluoromethyl)-1H-imidazol-1-yl]benzenesulfonamide;

g1) 2-(3,4-difluorophenyl)-1-[4-(methylsulfonyl)phenyl]-4-(trifluoromethyl)-1H-imidazole;

g2) 4-[2-(4-methylphenyl)-4-(trifluoromethyl)-1H-imidazol-1-yl]benzenesulfonamide;

g3) 2-(4-chlorophenyl)-1-[4-(methylsulfonyl)phenyl]-4-methyl-1H-imidazole;

g4) 2-(4-chlorophenyl)-1-[4-(methylsulfonyl)phenyl]-4-phenyl-1H-imidazole;

g5) 2-(4-chlorophenyl)-4-(4-fluorophenyl)-1-[4-(methylsulfonyl)phenyl]-1H-imidazole;

g6) 2-(3-fluoro-4-methoxyphenyl)-1-[4-(methylsulfonyl)phenyl-4-(trifluoromethyl)-1H-imidazole;

g7) 1-[4-(methylsulfonyl)phenyl]-2-phenyl-4-trifluoromethyl-1H-imidazole;

g8) 2-(4-methylphenyl)-1-[4-(methylsulfonyl)phenyl]-4-trifluoromethyl-1H-imidazole;

g9) 4-[2-(3-chloro-4-methylphenyl)-4-(trifluoromethyl)-1H-imidazol-1-yl]benzenesulfonamide;

g10) 2-(3-fluoro-5-methylphenyl)-1-[4-(methylsulfonyl)phenyl]-4-(trifluoromethyl)-1H-imidazole;

h1) 4-[2-(3-fluoro-5-methylphenyl)-4-(trifluoromethyl)-1H-imidazol-1-yl]benzenesulfonamide;

h2) 2-(3-methylphenyl)-1-[4-(methylsulfonyl)phenyl]-4-trifluoromethyl-1H-imidazole;

h3) 4-[2-(3-methylphenyl)-4-trifluoromethyl-1H-imidazol-1-yl]benzenesulfonamide;

h4) 1-[4-(methylsulfonyl)phenyl]-2-(3-chlorophenyl)-4-trifluoromethyl-1H-imidazole;

h5) 4-[2-(3-chlorophenyl)-4-trifluoromethyl-1H-imidazol-1-yl]benzenesulfonamide;

h6) 4-[2-phenyl-4-trifluoromethyl-1H-imidazol-1-yl]benzenesulfonamide;

h7) 4-[2-(4-methoxy-3-chlorophenyl)-4-trifluoromethyl-1H-imidazol-1-yl]benzenesulfonamide;

h8) 1-allyl-4-(4-fluorophenyl)-3-[4-(methylsulfonyl)phenyl]-5-(trifluoromethyl)-1H-pyrazole;

h9) 4-[1-ethyl-4-(4-fluorophenyl)-5-(trifluoromethyl)-1H-pyrazol-3-yl]benzenesulfonamide;

i1) N-phenyl-[4-(4-luorophenyl)-3-[4-(methylsulfonyl)phenyl]-5-(trifluoromethyl)-1H-pyrazol-1-yl]acetamide;

i2) ethyl [4-(4-fluorophenyl)-3-[4-(methylsulfonyl)phenyl]-5-(trifluoromethyl)-1H-pyrazol-1-yl]acetate;

i3) 4-(4-fluorophenyl)-3-[4-(methylsulfonyl)phenyl]-1-(2-phenylethyl)-1H-pyrazole;

i4) 4-(4-fluorophenyl)-3-[4-(methylsulfonyl)phenyl]-1-(2-phenylethyl)-5-(trifluoromethyl)pyrazole;

i5) 1-ethyl-4-(4-fluorophenyl)-3-[4-(methylsulfonyl)phenyl]-5-(trifluoromethyl)-1H-pyrazole;

i6) 5-(4-fluorophenyl)-4-(4-methylsulfonylphenyl)-2-trifluoromethyl-1H-imidazole;

i7) 4-[4-(methylsulfonyl)phenyl]-5-(2-thiophenyl)-2-(trifluoromethyl)-1H-imidazole;

i8) 5-(4-fluorophenyl)-2-methoxy-4-[4-(methylsulfonyl)phenyl]-6-(trifluoromethyl)pyridine;

i9) 2-ethoxy-5-(4-fluorophenyl)-4-[4-(methylsulfonyl)phenyl]-6-(trifluoromethyl)pyridine;

i10) 5-(4-fluorophenyl)-4-[4-(methylsulfonyl)phenyl]-2-(2-propynyloxy)-6-(trifluoromethyl)pyridine;

j1) 2-bromo-5-(4-fluorophenyl)-4-[4-(methylsulfonyl)phenyl]-6-(trifluoromethyl)pyridine;

j2) 4-[2-(3-chloro-4-methoxyphenyl)-4,5-difluorophenyl]benzenesulfonamide;

j3) 1-(4-fluorophenyl)-2-[4-(methylsulfonyl)phenyl]benzene;

j4) 5-difluoromethyl-4-(4-methylsulfonylphenyl)-3-phenylisoxazole;

j5) 4-[3-ethyl-5-phenylisoxazol-4-yl]benzenesulfonamide;

j6) 4-[5-difluoromethyl-3-phenylisoxazol-4-yl]benzenesulfonamide;

j7) 4-[5-hydroxymethyl-3-phenylisoxazol-4-yl]benzenesulfonamide;

j8) 4-[5-methyl-3-phenyl-isoxazol-4-yl]benzenesulfonamide;

j9) 1-[2-(4-fluorophenyl)cyclopenten-1-yl]-4-(methylsulfonyl)benzene;

j10) 1-[2-(4-fluoro-2-methylphenyl)cyclopenten-1-yl]-4-(methylsulfonyl)benzene;

k1) 1-[2-(4-chlorophenyl)cyclopenten-1-yl]-4-(methylsulfonyl)benzene;

k2) 1-[2-(2,4-dichlorophenyl)cyclopenten-1-yl]-4-(methylsulfonyl)benzene;

k3) 1-[2-(4-trifluoromethylphenyl)cyclopenten-1-yl]-4-(methylsulfonyl)benzene;

k4) 1-[2-(4-methylthiophenyl)cyclopenten-1-yl]-4-(methylsulfonyl)benzene;

k5) 1-[2-(4-fluorophenyl)-4,4-dimethylcyclopenten-1-yl]-4-(methylsulfonyl)benzene;

k6) 4-[2-(4-fluorophenyl)-4,4-dimethylcyclopenten-1-yl]benzenesulfonamide;

k7) 1-[2-(4-chlorophenyl)-4,4-dimethylcyclopenten-1-yl]-4-(methylsulfonyl)benzene;

k8) 4-[2-(4-chlorophenyl)-4,4-dimethylcyclopenten-1-yl]benzenesulfonamide;

k9) 4-[2-(4-fluorophenyl)cyclopenten-1-yl]benzenesulfonamide;

k10) 4-[2-(4-chlorophenyl)cyclopenten-1-yl]benzenesulfonamide;

l1) 1-[2-(4-methoxyphenyl)cyclopenten-1-yl]-4-(methylsulfonyl)benzene;

l2) 1-[2-(2,3-difluorophenyl)cyclopenten-1-yl]-4-(methylsulfonyl)benzene;

l3) 4-[2-(3-fluoro-4-methoxyphenyl)cyclopenten-1-yl]benzenesulfonamide;

l4) 1-[2-(3-chloro-4-methoxyphenyl)cyclopenten-1-yl]-4-(methylsulfonyl)benzene;

l5) 4-[2-(3-chloro-4-fluorophenyl)cyclopenten-1-yl]benzenesulfonamide;

l6) 4-[2-(2-methylpyridin-5-yl)cyclopenten-1-yl]benzenesulfonamide;

l7) ethyl 2-[4-(4-fluorophenyl)-5-[4-(methylsulfonyl)phenyl]oxazol-2-yl]-2-benzyl-acetate;

l8) 2-[4-(4-fluorophenyl)-5-[4-(methylsulfonyl)phenyl]oxazol-2-yl]acetic acid;

l9) 2-(tert-butyl)-4-(4-fluorophenyl)-5-[4-(methylsulfonyl)phenyl]oxazole;

l10) 4-(4-fluorophenyl)-5-[4-(methylsulfonyl)phenyl]-2-phenyloxazole;

m1) 4-(4-fluorophenyl)-2-methyl-5-[4-(methylsulfonyl)phenyl]oxazole; and

m2) 4-[5-(3-fluoro-4-methoxyphenyl)-2-trifluoromethyl-4-oxazolyl]benzenesulfonamide.

m3) 6-chloro-2-trifluoromethyl-2H-1-benzopyran-3-carboxylic acid;

m4) 6-chloro-7-methyl-2-trifluoromethyl-2H-1-benzopyran-3-carboxylic acid;

m5) 8-(1-methylethyl)-2-trifluoromethyl-2H-1-benzopyran-3-carboxylic acid;

m6) 6-chloro-7-(1,1-dimethylethyl)-2-trifluoromethyl-2H-1-benzopyran-3-carboxylic acid;

m7) 6-chloro-8-(1-methylethyl)-2-trifluoromethyl-2H-1-benzopyran-3-carboxylic acid;

m8) 2-trifluoromethyl-3H-naphthopyran-3-carboxylic acid;

m9) 7-(1,1-dimethylethyl)-2-trifluoromethyl-2H-1-benzopyran-3-carboxylic acid;

m10) 6-bromo-2-trifluoromethyl-2H-1-benzopyran-3-carboxylic acid;

n1) 8-chloro-2-trifluoromethyl-2H-1-benzopyran-3-carboxylic acid;

n2) 6-trifluoromethoxy-2-trifluoromethyl-2H-1-benzopyran-3-carboxylic acid;

n3) 5,7-dichloro-2-trifluoromethyl-2H-1-benzopyran-3-carboxylic acid;

n4) 8-phenyl-2-trifluoromethyl-2H-1-benzopyran-3-carboxylic acid;

n5) 7,8-dimethyl-2-trifluoromethyl-2H-1-benzopyran-3-carboxylic acid;

n6) 6,8-bis(dimethylethyl)-2-trifluoromethyl-2H-1-benzopyran-3-carboxylic acid;

n7) 7-(1-methylethyl)-2-trifluoromethyl-2H-1-benzopyran-3-carboxylic acid;

n8) 7-phenyl-2-trifluoromethyl-2H-1-benzopyran-3-carboxylic acid;

n9) 6-chloro-7-ethyl-2-trifluoromethyl-2H-1-benzopyran-3-carboxylic acid;

n10) 6-chloro-8-ethyl-2-trifluoromethyl-2H-1-benzopyran-3-carboxylic acid;

o1) 6-chloro-7-phenyl-2-trifluoromethyl-2H-1-benzopyran-3-carboxylic acid;

o2) 6,7-dichloro-2-trifluoromethyl-2H-1-benzopyran-3-carboxylic acid;

o3) 6,8-dichloro-2-trifluoromethyl-2H-1-benzopyran-3-carboxylic acid;

o4) 2-trifluoromethyl-3H-naptho[2,1-b]pyran-3-carboxylic acid;

o5) 6-chloro-8-methyl-2-trifluoromethyl-2H-1-benzopyran-3-carboxylic acid;

o6) 8-chloro-6-methyl-2-trifluoromethyl-2H-1-benzopyran-3-carboxylic acid;

o7) 8-chloro-6-methoxy-2-trifluoromethyl-2H-1-benzopyran-3-carboxylic acid;

o8) 6-bromo-8-chloro-2-trifluoromethyl-2H-1-benzopyran-3-carboxylic acid;

o9) 8-bromo-6-fluoro-2-trifluoromethyl-2H-1-benzopyran-3-carboxylic acid;

o10) 8-bromo-6-methyl-2-trifluoromethyl-2H-1-benzopyran-3-carboxylic acid;

p1) 8-bromo-5-fluoro-2-trifluoromethyl-2H-1-benzopyran-3-carboxylic acid;

p2) 6-chloro-8-fluoro-2-trifluoromethyl-2H-1-benzopyran-3-carboxylic acid;

p3) 6-bromo-8-methoxy-2-trifluoromethyl-2H-1-benzopyran-3-carboxylic acid;

p4) 6-[[(phenylmethyl)amino]sulfonyl]-2-trifluoromethyl-2H-1-benzopyran-3-carboxylic acid;

p5) 6-[(dimethylamino)sulfonyl]-2-trifluoromethyl-2H-1-benzopyran-3-carboxylic acid;

p6) 6-[(methylamino)sulfonyl]-2-trifluoromethyl-2H-1-benzopyran-3-carboxylic acid;

p7) 6-[(4-morpholino)sulfonyl]-2-trifluoromethyl-2H-1-benzopyran-3-carboxylic acid;

p8) 6-[(1,1-dimethylethyl)aminosulfonyl]-2-trifluoromethyl-2H-1-benzopyran-3-carboxylic acid;

p9) 6-[(2-methylpropyl)aminosulfonyl]-2-trifluoromethyl-2H-1-benzopyran-3-carboxylic acid;

p10) 6-methylsulfonyl-2-trifluoromethyl-2H-1-benzopyran-3-carboxylic acid;

q1) 8-chloro-6-[[(phenylmethyl)amino]sulfonyl]-2-trifluoromethyl-2H-1-benzopyran-3-carboxylic acid;

q2) 6-phenylacetyl-2-trifluoromethyl-2H-1-benzopyran-3-carboxylic acid;

q3) 6,8-dibromo-2-trifluoromethyl-2H-1-benzopyran-3-carboxylic acid;

q4) 8-chloro-5,6-dimethyl-2-trifluoromethyl-2H-1-benzopyran-3-carboxylic acid;

q5) 6,8-dichloro-(S)-2-trifluoromethyl-2H-1-benzopyran-3-carboxylic acid;

q6) 6-benzylsulfonyl-2-trifluoromethyl-2H-1-benzopyran-3-carboxylic acid;

q7) 6-[[N-(2-furylmethyl)amino]sulfonyl]-2-trifluoromethyl-2H-1-benzopyran-3-carboxylic acid;

q8) 6-[[N-(2-phenylethyl)amino]sulfonyl]-2-trifluoromethyl-2H-1-benzopyran-3-carboxylic acid;

q9) 6-iodo-2-trifluoromethyl-2H-1-benzopyran-3-carboxylic acid;

q10) 7-(1,1-dimethylethyl)-2-pentafluoroethyl-2H-1-benzopyran-3-carboxylic acid;

r1) 5,5-dimethyl-3-(3-fluorophenyl)-4-(4-methyl-sulphonyl-2(5H)-fluranone;

r2) 6-chloro-2-trifluoromethyl-2H-1-benzothiopyran-3-carboxylic acid;

r3) 4-[5-(4-chlorophenyl)-3-(trifluoromethyl)-1H-pyrazol-1-yl]benzenesulfonamide;

r4) 4-[5-(4-methylphenyl)-3-(trifluoromethyl)-1H-pyrazol-1-yl]benzenesulfonamide;

r5) 4-[5-(3-fluoro-4-methoxyphenyl)-3-(difluoromethyl)-1H-pyrazol-1-yl]benzenesulfonamide;

r6) 3-[1-[4-(methylsulfonyl)phenyl]-4-trifluoromethyl-1H-imidazol-2-yl]pyridine;

r7) 2-methyl-5-[1-[4-(methylsulfonyl)phenyl]-4-trifluoromethyl-1H-imidazol-2-yl]pyridine;

r8) 4-[2-(5-methylpyridin-3-yl)-4-(trifluoromethyl)-1H-imidazol-1-yl]benzenesulfonamide;

r9) 4-[5-methyl-3-phenylisoxazol-4-yl]benzenesulfonamide;

r10) 4-[5-hydroxymethyl-3-phenylisoxazol-4-yl]benzenesulfonamide;

s1) [2-trifluoromethyl-5-(3,4-difluorophenyl)-4-oxazolyl]benzenesulfonamide;

s2) 4-[2-methyl-4-phenyl-5-oxazolyl]benzenesulfonamide; or

s3) 4-[5-(3-fluoro-4-methoxyphenyl-2-trifluoromethyl)-4-oxazolyl]benzenesulfonamide;

or a pharmaceutically acceptable salt or prodrug thereof.

Cox-2 inhibitors that are useful in the methods and compositions of present invention can be supplied by any source as long as the Cox-2 inhibitor is pharmaceutically acceptable. Likewise, Cox-2 inhibitors that are useful in the compositions and methods of present invention can by synthesized, for example, according to the description in Example 1. Several Cox-2 inhibitors that are suitable for use with the compositions and methods of the present invention may be synthesized by the methods described in, for example, U.S. Pat. No. 5,466,823 to Talley, et al. Cox-2 inhibitors can also be isolated and purified from natural sources. Cox-2 inhibitors should be of a quality and purity that is conventional in the trade for use in pharmaceutical products.

Preferred Cox-2 selective inhibitor compounds are those compounds selected from the group consisting of celecoxib, parecoxib, deracoxib, valdecoxib, etoricoxib, meloxicam, rofecoxib, lumiracoxib, RS 57067, T-614, BMS-347070 (Bristol Meyers Squibb, described in U.S. Pat. No. 6,180,651), JTE-522 (Japan Tabacco), S-2474 (Shionogi), SVT-2016, CT-3 (Atlantic Pharmaceutical), ABT-963 (Abbott), SC-58125 (GD Searle), nimesulide, flosulide, NS-398 (Taisho Pharmaceutical), L-745337 (Merck), RWJ-63556, L-784512 (Merck), darbufelone (Pfizer), CS-502 (Sankyo), LAS-34475 (Almirall Prodesfarma), LAS-34555 (Almirall Prodesfarma), S-33516 (Servier), SD-8381 (Pharmacia, described in U.S. Pat. No. 6,0340256), MK-966 (Merck), L-783003 (Merck), T-614 (Toyama), D-1376 (Chiroscience), L-748731 (Merck), CGP-28238 (Novartis), BF-389 (Biofor/Scherer), GR-253035 (Glaxo Wellcome), prodrugs of any of them, and mixtures thereof.

More preferred is that the Cox-2 selective inhibitor is selected from the group consisting of celecoxib, parecoxib, deracoxib, valdecoxib, lumiracoxib, etoricoxib, rofecoxib, prodrugs of any of them, and mixtures thereof.

Even more preferred still is that the Cox-2 selective inhibitor is celecoxib.

Various classes of Cox-2 inhibitors useful in the present invention can be prepared as follows. Pyrazoles can be prepared by methods described in WO 95/15316. Pyrazoles can further be prepared by methods described in WO 95/15315. Pyrazoles can also be prepared by methods described in WO 96/03385.

Thiophene analogs useful in the present invention can be prepared by methods described in WO 95/00501. Preparation of thiophene analogs is also described in WO 94/15932.

Oxazoles useful in the present invention can be prepared by the methods described in WO 95/00501. Preparation of oxazoles is also described in WO 94/27980.

Isoxazoles useful in the present invention can be prepared by the methods described in WO 96/25405.

Imidazoles useful in the present invention can be prepared by the methods described in WO 96/03388. Preparation of imidazoles is also described in WO 96/03387.

Cyclopentene Cox-2 inhibitors useful in the present invention can be prepared by the methods described in U.S. Pat. No. 5,344,991. Preparation of cyclopentene Cox-2 inhibitors is also described in WO 95/00501.

Terphenyl compounds useful in the present invention can be prepared by the methods described in WO 96/16934.

Thiazole compounds useful in the present invention can be prepared by the methods described in WO 96/03,392.

Pyridine compounds useful in the present invention can be prepared by the methods described in WO 96/03392. Preparation of pyridine compounds is also described in WO 96/24,585.

Benzopyranopyrazolyl compounds useful in the present invention can be prepared by the methods described in WO 96/09304.

Chromene compounds useful in the present invention can be prepared by the methods described in WO 98/47890. Preparation of chromene compounds is also described in WO 00/23433. Chromene compounds can further be prepared by the methods described in U.S. Pat. No. 6,077,850. Preparation of chromene compounds is further described in U.S. Pat. No. 6,034,256.

Arylpyridazinones useful in the present invention can be prepared by the methods described in WO 00/24719. Preparation of arylpyridazinones is also described in WO 99/10332. Arylpyridazinones can further be prepared by the methods described in WO 99/10331.

5-Alkyl-2-arylaminophenylacetic acids and derivatives useful in the present invention can be prepared by the methods described in WO 99/11605.

Diarylmethylidenefuran derivative Cox-2 selective inhibitors useful in the present invention can be prepared by the methods described in U.S. Pat. No. 6,180,651.

The celecoxib used in the compositions and methods of the present invention can be prepared in the manner set forth in U.S. Pat. No. 5,466,823.

The valdecoxib used in the compositions and methods of the present invention can be prepared in the manner set forth in U.S. Pat. No. 5,633,272.

The parecoxib used in the compositions and methods of the present invention can be prepared in the manner set forth in U.S. Pat. No. 5,932,598.

The rofecoxib used in the compositions and methods of the present invention can be prepared in the manner set forth in U.S. Pat. No. 5,474,995.

The deracoxib used in the compositions and methods of the present invention can be prepared in the manner set forth in U.S. Pat. No. 5,521,207.

The etoricoxib used in the compositions and methods of the present invention can be prepared in the manner set forth in WO 98/03484.

The meloxicam used in the compositions and methods of the present invention can be prepared in the manner set forth in U.S. Pat. No. 4,233,299.

The compound 4-(4-cyclohexyl-2-methyloxazol-5-yl)-2-fluorobenzenesulfonamide used in the compositions and methods of the present invention can be prepared in the manner set forth in U.S. Pat. No. 5,994,381.

The compound 2-(3,4-difluorophenyl)-4-(3-hydroxy-3-methylbutoxy)-5-[4-(methylsulfonyl)phenyl]-3(2H)-pyridazinone used in the compositions and methods of the present invention can be prepared in the manner set forth in WO 00/24719.

The compound 2-(3,5-difluorophenyl)-3-[4-(methylsulfonyl)phenyl]-2-cyclopenten-1-one used in the compositions and methods of the present invention can be prepared in the manner set forth in EP 863134.

The compound 2-[(2-chloro-6-fluorophenyl)amino]-5-methyl-benzeneacetic acid used in the compositions and methods of the present invention can be prepared in the manner set forth in WO 99/11605.

The compound N-[2-(cyclohexyloxy)-4-nitrophenyl]methanesulfonamide used in the compositions and methods of the present invention can be prepared in the manner set forth in U.S. Pat. No. 4,885,367.

The compound (3Z)-3-[(4-chlorophenyl)[4-(methylsulfonyl)phenyl]methylene]dihydro-2(3H)-furanone used in the compositions and methods of the present invention can be prepared in the manner set forth in U.S. Pat. No. 6,180,651.

Another element of the present invention is a 5-HT_{1A} receptor modulator. The expression “5-HT_{1A} receptor” refers to the 5-hydroxytryptamine_{1A} receptor, which is pharmacologically characterized by its high affinity for 5-hydroxytryptamine (5-HT, serotonin). The expression “5-HT_{1A} receptor” refers to proteins having amino acid sequences which are substantially similar to native mammalian 5-hydroxytryptamine_{1A} receptors or 5-hydroxytryptamine_{1A} amino acid sequences, and which are capable of binding 5-hydroxytryptamine molecules and inhibiting 5-hydroxytyryptamine from binding to the 5-hydroxytryptamine_{1A} receptor. The human 5-HT_{1A} receptor is located on chromosome 5q11.2-q13 and has 422 amino acids.

The expression “5-HT_{1A} receptor modulator” refers to a compound that binds to the 5-HT_{1A} receptor and modulates its activity, with, for example, agonist, reverse agonist, antagonist or mixed effects.

The structures of some examples of preferred 5-HT_{1A} receptor modulators are listed in Table No. 3 below.

TABLE 3
Examples of 5-HT_{1A} Receptor Modulators

| Compound | |
|---|---|
| Number | Structure |
| H1 | 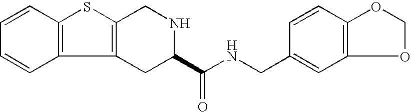 |
| H2 | 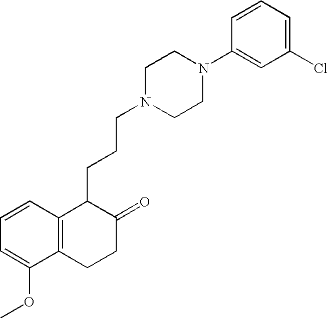 |
| H3 | 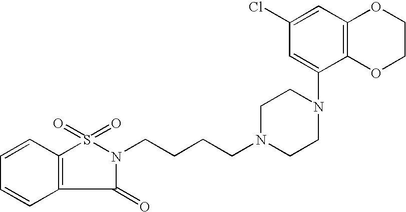 |
| H4 | 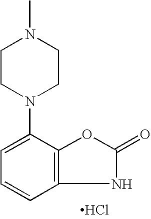 |
| H5 | 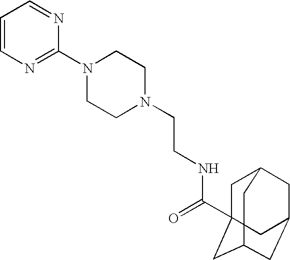 |
| H6 | 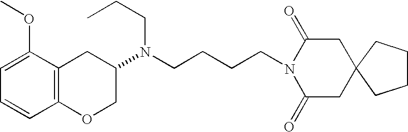 |
| H7 | 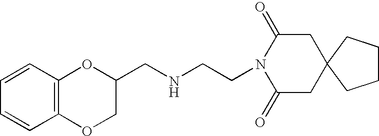 |
| H8 | 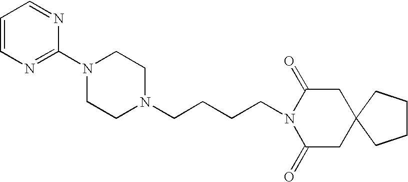 |
| H10 | 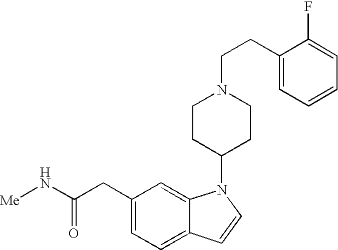 |
| H11 | 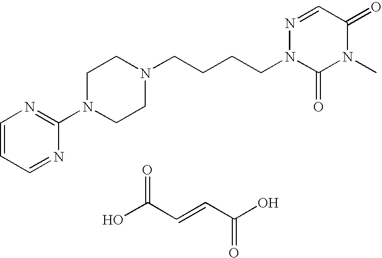 |
| H12 | 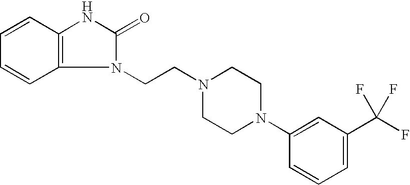 |
| H13 | 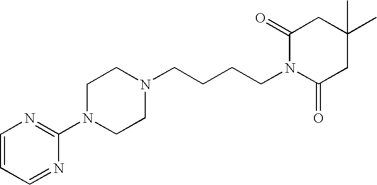 |
| H14 | 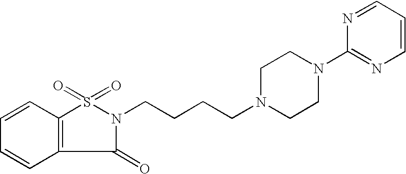 |
| H15 | 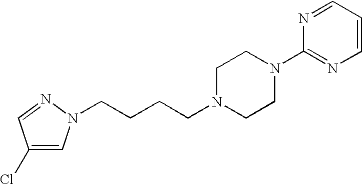 |
| H16 | 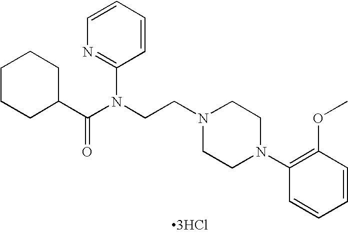 |
| H17 | 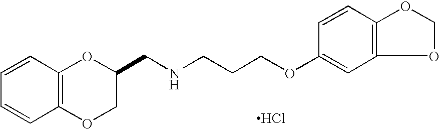 |
| H18 | 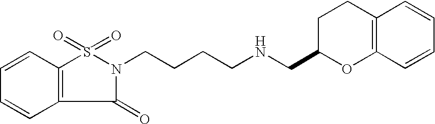 |
| H19 | 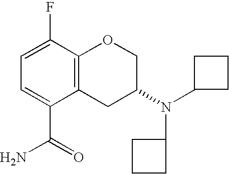 |
| H20 | 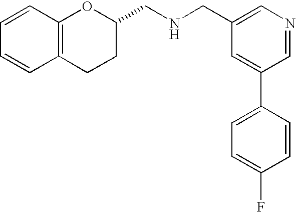 |
| H22 | 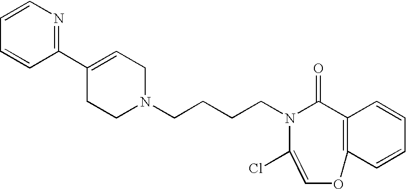 |
| H23 | 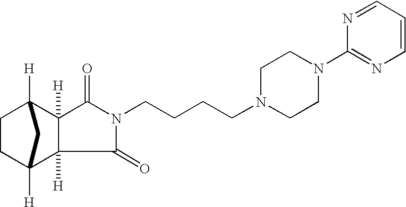 |
| H24 | 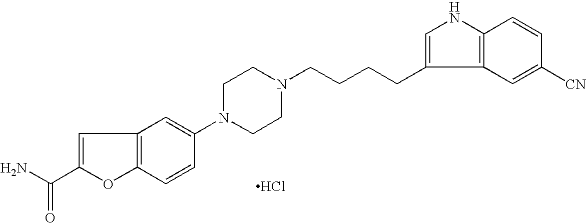 |
| H26 | 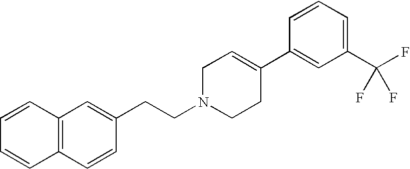 |
| H27 | 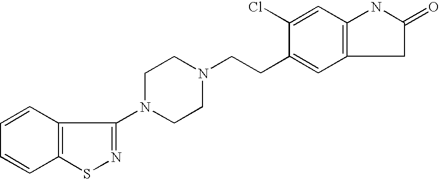 |

The names, CAS registry numbers and references for examples of preferred 5-HT_{1A} receptor modulators are listed in Table 4, below.

TABLE 4
Examples of preferred 5-HT_{1A} Receptor Modulator Names, CAS
Registry Numbers and References

| Compound | | CAS Registry | |
|---|---|---|---|
| Number | Name(s) | Number | Reference |
| H1 | (R)-N-(1,3-benzodioxol-5- | 151227-58-6 | JP07109281 |
| | ylmethyl)-1,2,3,4-tetrahydro- | | |
| | [1]benzothieno[2,3- | | |
| | c]pyridine-3-carboxamide, | | |
| | (AP-521) | | |
| H2 | 1-[3-[4-(3-chlorophenyl)-1- | 145969-30-8 | EP512525 |
| | piperazinyl]propyl]-3,4- | | |
| | dihydro-5-methoxy-2(1H)- | | |
| | quinolinone (OPC-14523) | | |
| H3 | 2-[4-[4-(7-chloro-2,3-dihydro- | 161611-99-0 | |
| | 1,4-benzodioxin-5-yl)-1- | | |
| | piperazinyl]butyl]-1,2- | | |
| | benzisothiazol-3(2H)-one, | | |
| | 1,1-dioxide (DU-125530) | | |
| H4 | 7-(4-methyl-1-piperazinyl)- | 269718-83-4 | WO0029397 |
| | 2(3H)benzoxazolone, | | |
| | monohydrochloride (SLV- | | |
| | 308) | | |
| H5 | adatanserin | 127266-56-2 | US5010078 |
| H6 | alnespirone | 138298-79-0 | EP452204 |
| H7 | binospirone | 102908-59-8 | EP170213 |
| H8 | buspirone | 36505-84-7 | US3717634 |
| H9 | DU-127090 | 362524-71-8 | |
| H10 | 2-[1-[1-[2-(2- | | WO9843956 |
| | fluorophenyl)ethyl]piperidino- | | |
| | 4-yl]-1H-indol-6-yl]-N- | | |
| | methylacetamide (E-2101) | | |
| H11 | eptapirone | 179756-85-5 | WO09616949 |
| H12 | flibanserin | 167933-07-5 | EP526434 |
| H13 | gepirone | 83928-76-1 | US4423049 |
| H14 | ipsapirone | 95847-70-4 | DE3321969 |
| H15 | lesopitron | 132449-46-8 | EP382637 |
| H16 | N-[2-[4-(2-methoxyphenyl)-1- | 146714-97-8 | EP512755 |
| | piperazinyl]ethyl]-N-2- | | |
| | pyridinyl- | | |
| | cyclohexanecarboxamide, | | |
| | trihydrochloride (WAY- | | |
| | 100635) | | |
| H17 | N-[3-(1,3-benzodioxol-5- | 137275-80-0 | EP452204 |
| | yloxy)propyl]-2,3-dihydro- | | |
| | (2S)-1,4-benzodioxin-2- | | |
| | methanamine, hydrochloride | | |
| | (MKC-242) | | |
| H18 | repinotan (BAY X3702) | 144980-29-0 | EP749970 |
| H19 | robalzotan | 169758-66-1 | WO09511891 |
| H20 | sarizotan | 177975-08-5 | EP707007 |
| H21 | SLV-319 | | |
| H22 | SUN-N4057 | 182415-09-4 | WO9624594 |
| H23 | tandospirone | 87760-53-0 | EP82402 |
| H24 | vilazodone | 163521-08-2 | EP648767 |
| H25 | VML-670 | | |
| H26 | xaliproden | 135354-02-8 | EP101381 |
| H27 | ziprasidone | 146939-27-7 | US4883795 |
| H28 | 6-hydroxy-buspirone | | US6150365 |
| H29 | pyrazolidine derivative | | EP736526 |
| H30 | Heteroaryloxyethylamines | | US6063784 |
| H31 | 5-hydoxytryptamine, 5- | | WO9210200 |
| | methoxytryptamine, | | |
| | buspirone, 8-hydroxy-2-(di-n- | | |
| | propylamino) tetralin (8-OH- | | |
| | DPAT), ipsaspirone, | | |
| | gepirone, SM23997, lysergic | | |
| | acid, diethylamide, and | | |
| | agonistic antibodies | | |
| H32 | piperazine derivatives | | WO9311122 |
| H33 | 8-(2-aminoalkoxy) | | WO9429293 |
| | fluorochroman derivative | | |
| H34 | abeo-ergoline derivatives | | WO9528403 |
| H35 | A-74283 | 131818-91-2 | |
| H36 | AP-159 | 129592-83-2 | |
| H37 | AZ 16596 | 257864-15-6 | |
| H38 | 2-[[4-(2- | | |
| | Methoxyphenyl)piperazin-1- | | |
| | yl] methyl] octahydroimidazo | | |
| | [1,5-a] pyridine-1,3-dione | | |
| | (B 20991) | | |
| H39 | BMS 181100 (BMY 14802) | 105565-56-8 | |
| H40 | BMS 181101 (BMY 42569) | 146479-45-0 | DE03507983 |
| H41 | BMS 181970 | | |
| H42 | 1-methyl-4-[7-(4- | | |
| | chlorophenyl)methylaminocarbonyl] | | |
| | napththyl-piperazine | | |
| | (CP291952) | | |
| H43 | (omega- | | JP57080379 |
| | piperazinylalkoxy)alkylenedioxybenzene | | |
| | (BP 554) | | |
| H44 | E 5165 | | |
| H45 | E 6265 | | |
| H46 | Ebalzotan | 149494-37-1 | |
| H47 | Eltoprazine | 98224-03-4 | |
| H48 | F 11440 | 179756-58-2 | |
| H49 | F 13714 | | |
| H50 | Flesinoxan | | EP00138280 |
| H51 | 2-[4-(3-Phenylpyrrolidin-1- | | |
| | yl)butyl]- | | |
| | 1,2-benzisothiazol-3(2H)- | | |
| | one 1,1-dioxide (LB 50016) | | |
| H52 | LY 41 | 140221-50-7 | |
| H53 | (+/−)-4-Substituted-amino-6- | 115994-79-0 | EP00153083 |
| | substituted-1,3,4,5- | | |
| | tetrahydrobenz[c,d]inoles | | |
| | (LY 228729) | | |
| H54 | LY 228730 | | |
| H55 | LY 274600 | 132873-35-9 | |
| H56 | LY 274601 | 132873-34-8 | |
| H57 | LY 293284 | 141318-62-9 | |
| H58 | 6-Heterocyclyl-4-amino- | 156896-33-2 | EP00590971 |
| | 1,3,4,5-tetrahydrobenz CD | | |
| | indoles (LY 297996) | | |
| H59 | Isoxazole derivatives | | US5434174 |
| | (LY 315535) | | |
| H60 | hetero-oxy alkanamines | | EP00722941 |
| | (LY 333068) | | |
| H61 | LY 426965 | 326821-27-6 | |
| H62 | LY 433221 | | |
| H63 | MDL 72832 | 113777-33-6 | |
| H64 | MDL 73975 | 159650-30-3 | |
| H65 | NDL 249 | 169758-71-8 | |
| | | 184675-01-2 | |
| H66 | Nerisopam | 102771-12-0 | |
| H67 | Org 1301 | 142494-12-0 | |
| H68 | 2-(2-oxo- | | |
| | hexahydropyrimidin-1- | | |
| | yl)propylaminomethyl- | | |
| | benzopyran (R 137696) | | |
| H69 | RU 24969 | 66611-26-5 | |
| | | 107008-28-6 | |
| H70 | 1-[[5-[[4-substituted-1- | | US4992441 |
| | piperazinyl]methyl]-pyrrol-2- | | |
| | yl or furan-2-yl]methyl-2- | | |
| | piperidinones (RWJ 25730) | | |
| H71 | S 14489 | 153607-44-4 | |
| H72 | 1-Naphthyl-piperazine | 135721-98-1 | EP00434561 |
| | derivatives (S 14506) | | |
| H73 | 1-Naphthyl-piperazine | 135722-27-9 | EP00434561 |
| | derivatives (S 14671) | | |
| H74 | S 15535 | 146998-34-7 | |
| H75 | S 15931 | 153607-45-5 | |
| H76 | 8-[4-[N-(5-Acetyl-3,4- | | |
| | dihydro-2H-1-benzopyran-3- | | |
| | yl)-Npropylamino] | | |
| | butyl]-8-azaspiro [4.5] | | |
| | decane-7,9-dione (S 23751) | | |
| H77 | SDZ 216-525 | 141533-35-9 | |
| H78 | SEP 109235 | | |
| H79 | SR 59026 | | |
| H80 | Sunepitron | 131744-27-9 | |
| H81 | UH 301 | 127126-21-0 | |
| | | 135308-68-8 | |
| | | 187593-75-5 | |
| H82 | WAY 100135 | 133025-23-7 | |
| H83 | WAY 100802 | | |
| H84 | Zalospirone | | GB02181731 |

Also useful as a 5-HT_{1A} receptor modulator in the present invention is [(3-chloro-4-fluoro-phenyl)-[4-fluoro-4-{[(5-methyl-pyridin-2-ylmethyl)amino]-methyl)piperidin-1-yl]-methadone] (F 13640), as described in Colpaert, F. C. et al, Neuropharmacology, 43:945-958 (2002).

Especially preferred 5-HT_{1A} receptor modulators for the present invention include buspirone, gepirone, repinotan, tandospirone, xaliproden and ziprasidone.

The compounds useful in the present invention optiontionally can have no asymmetric carbon atoms, or, alternatively, the useful compounds can have one or more asymmetric carbon atoms. When the useful compounds have one or more asymmetric carbon atoms, they therefore include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention.

Also included in the methods, combinations and compositions of the present invention are the isomeric forms and tautomers of the described compounds and the pharmaceutically-acceptable salts thereof. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

Suitable pharmaceutically-acceptable base addition salts of compounds of the present invention include metallic ion salts and organic ion salts. More preferred metallic ion salts include, but are not limited to appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

Also included in the methods, combinations and compositions of the present invention are the prodrugs of the described compounds and the pharmaceutically-acceptable salts thereof. The term “prodrug” refers to drug precursor compounds which, following administration to a subject and subsequent absorption, are converted to an active species in vivo via some process, such as a metabolic process. Other products from the conversion process are easily disposed of by the body. More preferred prodrugs produce products from the conversion process that are generally accepted as safe. A nonlimiting example of a “prodrug” that will be useful in the methods, combinations and compositions of the present invention is parecoxib (N-[[4-(5-methyl-3-phenyl-4-isoxazolyl)phenyl]sulfonyl]propanamide), which is a prodrug for valdecoxib.

The methods and combinations of the present invention are useful for the treatment or prevention of pain, inflammation, or inflammation-related disorder. In a preferred embodiment, the subject is one that is in need of treatment or prevention of pain, inflammation, or an inflammation-related disorder.

The methods and combinations of the present invention are also useful for the treatment or prevention of neurologic disease involving neurodegeneration.

The phrase “combination therapy” (or “co-therapy”) embraces the administration of a Cox-2 inhibitor and a 5-HT_{1A} receptor modulator as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form, such as a capsule, for example, having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies.

The phrase “therapeutically effective” is intended to qualify the amount of compounds utilized in the therapy. This amount will achieve the goal of treating or preventing pain, inflammation or an inflammation-related disorder.

“Therapeutic compound” means a compound useful in the treatment or prevention of pain, inflammation or an inflammation-related disorder, or of a neurologic disorder involving neurodegeneration.

The term “pharmaceutically acceptable” is used adjectivally herein to mean that the modified noun is appropriate for use in a pharmaceutical product. Pharmaceutically acceptable cations include metallic ions and organic ions. More preferred metallic ions include, but are not limited to appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

The term “comprising” means “including the following elements but not excluding others.”

Combinations and Methods:

Among its several embodiments, the present invention provides a composition comprising Cox-2 inhibitor and a 5-HT_{1A} receptor modulator. It is preferred that the composition provides an amount of the Cox-2 inhibitor and an amount of the 5-HT_{1A} receptor modulator wherein the amount of the Cox-2 inhibitor and the amount of the 5-HT_{1A} receptor modulator together comprise a therapeutically effective amount for the treatment or prevention of pain, inflammation, or an inflammation-related disorder, and for the treatment or prevention of a neurologic disorder involving neurodegeneration.

In one embodiment, the Cox-2 inhibitor compound is a non-steroidal anti-inflammatory drug.

In another embodiment, the Cox-2 inhibitor is a Cox-2 selective inhibitor.

In still another embodiment, the the Cox-2 inhibitor compound is a prodrug of a Cox-2 inhibitor compound, illustrated herein with parecoxib.

In yet another embodiment, the present invention further provides a combination therapy method for the treatment or prevention of pain, inflammation, or an inflammation-related disorder in a mammal in need thereof, comprising administering to the mammal an amount of a Cox-2 inhibitor and an amount of a 5-HT_{1A} receptor modulator wherein the amount of the Cox-2 inhibitor and the amount of the 5-HT_{1A} receptor modulator together comprise a therapeutically effective amount for the treatment or prevention of pain, inflammation, or an inflammation-related disorder.

In a further embodiment, the present invention provides a pharmaceutical composition for the treatment or prevention of pain, inflammation, or an inflammation-related disorder comprising an amount of a Cox-2 inhibitor and an amount of a 5-HT_{1A} receptor modulator and a pharmaceutically-acceptable excipient.

In still further embodiment, the present invention provides a kit that is suitable for use in the treatment, prevention or inhibition of pain, inflammation, or an inflammation-related disorder wherein the kit comprises a first dosage form comprising a Cox-2 inhibitor and a second dosage form comprising a 5-HT_{1A} receptor modulator, in quantities which comprise a therapeutically effective amount of the compounds for the treatment, prevention or inhibition of pain, inflammation, or an inflammation-related disorder.

The methods and compositions of the present invention provide one or more benefits. Combinations of Cox-2 inhibitors and 5-HT_{1A} receptor modulators are useful in treating and preventing pain, inflammation, or an inflammation-related disorder. Preferably, the Cox-2 inhibitors and the 5-HT_{1A} receptor modulators of the present invention are administered in combination at a low dose, that is, at a dose lower than has been conventionally used in clinical situations.

The combinations of the present invention will have a number of uses. For example, through dosage adjustment and medical monitoring, the individual dosages of the therapeutic compounds used in the combinations of the present invention will be lower than are typical for dosages of the therapeutic compounds when used in monotherapy. The dosage lowering will provide advantages including reduction of side effects of the individual therapeutic compounds when compared to the monotherapy. In addition, fewer side effects of the combination therapy compared with the monotherapies will lead to greater subject compliance with therapy regimens.

Alternatively, the methods and combination of the present invention can also maximize the therapeutic effect at higher doses.

When administered as a combination, the therapeutic agents can be formulated as separate compositions that are given at the same time or different times, or the therapeutic agents can be given as a single composition.

Inhibitors of the cyclooxygenase pathway in the metabolism of arachidonic acid that are used in the treatment, prevention or reduction of pain, inflammation, or an inflammation-related disorder may inhibit enzyme activity through a variety of mechanisms. By way of example, the cyclooxygenase-2 inhibitors used in the methods described herein may block the enzyme activity directly by binding at the substrate site of the enzyme. The use of a Cox-2 selective inhibiting agent is highly advantageous in that it minimizes the gastric side effects that can occur with non-selective non-steroidal antiinflammatory drugs (NSAIDs), especially where prolonged treatment is expected.

Besides being useful for human treatment, these methods are also useful for veterinary treatment of companion animals, exotic animals and farm animals, including mammals, rodents, avians, and the like. More preferred animals include horses, dogs, and cats.

As used herein, the terms “therapeutically effective amount” are intended to qualify the amount of a Cox-2 inhibiting agent and a 5-HT_{1A} receptor modulator that are required to treat or prevent pain, inflammation, or an inflammation-related disorder, or to treat or prevent neurologic disease involving neurodegeneration.

The combinations and methods of the present invention would be useful for, but not limited to, the treatment of inflammation in a subject, and for treatment of other inflammation-related disorders, such as, as an analgesic in the treatment of pain and headaches, or as an antipyretic for the treatment of fever. For example, compounds of the invention would be useful to treat arthritis, including but not limited to rheumatoid arthritis, spondyloarthropathies, gouty arthritis, osteoarthritis, systemic lupus erythematosus and juvenile arthritis.

Such combinations and methods of the invention would be useful in the treatment of asthma, bronchitis, menstrual cramps, overactive bladder (OAB), preterm labor, tendinitis, bursitis, allergic neuritis, cytomegalovirus infectivity, apoptosis including HIV induced apoptosis, lumbago, liver disease including hepatitis, skin-related conditions such as psoriasis, eczema, acne, UV damage, burns and dermatitis, and postoperative inflammation including ophthalmic surgery such as cataract surgery and refractive surgery.

Combinations and methods of the invention also would be useful to treat gastrointestinal conditions such as inflammatory bowel disease, Crohn's disease, gastritis, irritable bowel syndrome and ulcerative colitis.

Combinations and methods of the invention would be useful in treating inflammation in such diseases as migraine headaches, polyarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

The combinations and methods of the invention would also be useful in the treatment of ophthalmic diseases, such as retinitis, conjunctivitis, retinopathies, uveitis, ocular photophobia, glaucoma and acute injury to the eye tissue.

The combinations and methods would also be useful in the treatment of pulmonary inflammation, such as that associated with viral infections and cystic fibrosis, and in bone resorption such as associated with osteoporosis.

The combinations and methods of the invention are useful as anti-inflammatory agents, such as for the treatment of arthritis, with the additional benefit of having significantly less harmful side effects. These combinations and methods would also be useful in the treatment of allergic rhinitis, respiratory distress syndrome, endotoxin shock syndrome, and liver disease.

The combinations and methods would also be useful in the treatment of pain, but not limited to postoperative pain, dental pain, muscular pain, dysmennorrhea, neuropathic pain and pain resulting from cancer.

The combinations and methods above would be useful for, but not limited to, treating and preventing inflammation-related cardiovascular disorders in a subject. The combinations and methods would be useful for treatment and prevention of vascular diseases, coronary artery disease, aneurysm, vascular rejection, arteriosclerosis, atherosclerosis including cardiac transplant atherosclerosis, myocardial infarction, embolism, stroke (hemorrhagic or ischemic), thrombosis, including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

The combinations and methods would be useful for, but not limited to, the treatment and prevention of angiogenesis-related disorders in a subject. According to the present invention, the compounds can be administered to a subject in need of angiogenesis inhibition. The method would be useful for treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and glaucoma; ulcerative diseases such as gastric ulcer; pathological, but non-malignant, conditions such as hemangiomas, including infantile hemaginomas, angiofibroma of the nasopharynx and avascular necrosis of bone; and disorders of the female reproductive system such as endometriosis.

Compounds of the invention would be useful for the prevention or treatment of benign and malignant tumors/neoplasia including cancer, such as colorectal cancer, brain cancer, bone cancer, epithelial cell derived neoplasia (epithelial carcinoma) such as basal cell carcinoma, adenocarcinoma, gastrointestinal cancer such as lip cancer, mouth cancer, esophogeal cancer, small bowel cancer and stomach cancer, colon cancer, liver cancer, bladder cancer, pancreas cancer, ovarian cancer, cervical cancer, lung cancer, breast cancer and skin cancer, such as squamous cell and basal cell cancers, prostate cancer, renal cell carcinoma, and other known cancers that effect epithelial cells throughout the body. Preferably, neoplasia is selected from gastrointestinal cancer, Barrett's esophagus, liver cancer, bladder cancer, pancreas cancer, ovarian cancer, prostate cancer, cervical cancer, lung cancer, breast cancer and skin cancer, such as squamus cell and basal cell cancers. The compounds can also be used to treat the fibrosis which occurs with radiation therapy. The method can be used to treat subjects having adenomatous polyps, including those with sporadic adenomatous polyposis (SAP) or familial adenomatous polyposis (FAP). Additionally, the method can be used to prevent polyps from forming in subjects at risk of FAP.

The combinations and methods of the present invention are useful for the prevention and treatment of pain, inflammation and central nervous system (CNS) disorders, which include, for example, adjustment disorders, such as anxiety (mixed anxiety), mood (depressed), conduct disturbance, mixed anxiety and mood (conduct); addictive disorders, such as alcohol abuse, intoxication disorders, nicotine abuse, psychoactive substances abuse and substance disorder; withdrawal syndromes; acute trauma; age associated mental disorders, such as learning and Alzheimer's disease; agitation disorders, such as agitation in Alzheimer's disease and agitation in the elderly; aggressive behavior, such as in Alzheimers disease; amyloidosis, such as aging/senile, hereditary, immunocyte derived, lichen, primary, reactive systemic, secondary, senile (Alzheimer's disease), amyotrophy & amyotropic lateral sclerosis (ALS), and anorexia nervosa; anxiety disorders, such as generalized anxiety disorder (GAD), panic disorder, bipolar disorder, social phobias and stress related diseases; apathy; attention deficit disorder (ADD); attention deficit hyperactivity disorder (ADHD); autism; auto immune disorders, such as lupus erythematosis and multiple sclerosis; behavioral disturbances, such as agitation plus diminished cognition, bipolar I disorder and bipolar II disorder; bulimia nervosa; cardiovascular; blood pressure modification, such as for hypertension, hypotension and heart rate modification; chemotherapy-induced vomiting; chronic fatigue immune disorders (CFIDS); chronic fatigue syndrome (CFS); cognitive dysfunction, such as cortical dementias including mild cognitive impairment (MCI), Lewy Body dementia, vascular dementia, neurodegeneration, and cognitive dysfunction resulting from stroke, ischemia, trauma, or surgical procedures, including coronary artery bypass surgery; cognition enhancement; conduct disorder; cyclothymia; delusional disorder; depression, such as adolescent, in Alzheimer's disease, general, minor, in Parkinson's disease and diabetic neuropathy; dissociative disorders; developmental disorders, such as learning disabilities, language disorders and mental retardation; dementia, such as associated with aging, illness, neurodegeneration and dyskensia; dysthymia; dystonia; eating disorders, such as anorexia nervosa, bulimia nervosa, obesity, epilepsy and fibromyalgia syndrome (FMS); gastrointestinal disorders, such as irritable bowel syndrome, psychogenic effects and stress-related; growth retardation effects, such as endocrine, psychosocial and stress-related; heart rate modification; Huntington's Chorea; hypertension; immune system disorders, such as immune system depression; impulse control (related to conduct disorder); incontinence, such as mixed states, stress incontinence and urge incontinence; infectious neuropathy, such as AIDS; carpal tunnel syndrome; dementia; irritable bowel syndrome (IBS), such as constipative and diarrhea-predominant; inflammatory bowel disease (IBD), such as constipation-predominant, diarrhea-predominant and mixed states; inhalation disorder; lactation inhibition, metabolic & chromosomal disorders, such as galactosemia phenylketonuria, fatty acid disorder, infantile nephropathic cystinosis, orthithrotranscarbamylase porphyria and migrane; mood disorders, such as a typical depression, bipolar disorder (including pychotic features), major depressive disorder, mania, and seasonal affective disorder; movement disorders, such as athetosis, chorea, dyskinesia, dystonia, restless leg syndrome (RLS), tremor plus periodic limb movement (PLM), periodic limb movements of sleep (PLMS), Huntington's chorea, Parkinson's disease, PLM, PLMS, progressive supranuclear palsy, stereotypy (various), torticollis, tic disorders and tremor; multisystemic atrophy (MSA), such as multiple sclerosis; neuroendocrine system disorders; neurologic diseases involving neurodegeneration, such as amyotrophy, amyotrophy diabetics, amyotrophic lateral sclerosis (ALS), Alzheimer's disease, Huntington's chorea and Parkinson's disease; neurological disorders; neuropathy, such as diabetic and peripheral; neuroprotective effects, such as for ischemic brain injury, myocardial infarction, spinal cord injury, traumatic brain injury and obesity; obsessive compulsive disorder (OCD); oncology related disorders, such as with behavior abnormalities resulting from tumors or treatments, such as chemotherapy and induced vomiting; oppositional defiant disorder; pain disorders, such as acute, chronic, cluster headache, dysmenorrhea, labor, migraine, neuropathic, AIDs-related, cancer-related, chemotherapeutic-induced, diabetic, post-herpetic neuralgia, radiation-induced, osteoarthritis flare, phantom limb, surgical, post-surgical and incisional, psychic, regional pain (such as abdominal region, chronic back pain, complex-regional pain disorder, dental, face and mouth, head, lower back and peripheral), rheumatoid arthritis, starting pain and systematic pain (such as in connective tissue, such as musculoskeletal, nervous system, urogenital, uterine contractions); panic disorder, such as with agoraphobia and without agoraphobia; Parkinson's disease; peripheral neuropathy; personality disorders; phobias (simple), such as phobias of animals, closed spaces (claustrophobia), heights (acrophobia), public places (agoraphobia); phobias (Social), such as, public eating, public embarrassment, public performance/speaking and using public lavatories; SSRI poop out syndrome; post-traumatic stress disorder; progressive supranuclear palsy (PSP); prolactin plasma level disorders; psychotic disorders, such as brief and long duration, due to medical condition, not otherwise specified (NOS) and restless leg syndrome (RLS); schizophrenias, such as delusional (paranoid) disorder, schizoaffective disorders, schizophreniform disorders and seasonal affective disorder; seizure disorders, such as epilepsy (partial) and epilepsy (generalized); sexual dysfunction, such as for female and for male; sleep disorders, such as apnea, parasomnias, insomnia, narcolepsy, obstructive, and disorders of circadian rhythm, enuresis, initiation and maintenance; social phobias, such as social anxiety disorder; somatoform disorders, such as conversion, body, dysmorphic, fibromyalgia syndrome (FMS), hypochondriasis, NOS, somatization and undifferentiated; specific developmental disorders; stress disorders, such as acute, chronic and incontinence; spectrum disorders; stroke; suicidal behavior, and in particular, prevention of and amelioration of; thyroid stimulating hormone disorders (TSH); Tourette's syndrome; tooth-germ morphogenesis disorders; thermoregulation disorders; TSH modulating agent disorders; tic disorders; trauma, such as acute head, and related effects, such as blood pressure regulation, cerebral blood flow, CSF production, inflammation, and ischemia; vasospasms; vasoreactive headaches and violent behavior.

As used herein, the term “treatment” includes partial or total inhibition of the dementia or cognitive dysfunction, including Alzheimer's disease, vascular dementia, multi-infarct dementia, pre-senile dementia, alcoholic dementia, and senile dementia.

The combinations and methods of the present invention are particularly useful for the treatment, prevention or inhibition of a central nervous system disorder associated with stroke (ischemic or hemmorhagic) or other ischemic brain injury.

The phrases “low dose” or “low dose amount”, in characterizing a therapeutically effective amount of the Cox-2 selective inhibitor and the 5-HT_{1A} receptor modulator or therapy in the combination therapy, defines a quantity of such agent, or a range of quantity of such agent, that is capable of reducing the discomfort of pain, inflammation, or an inflammation-related disorder while optionally reducing or avoiding one or more side effects of monotherapy with a 5-HT_{1A} receptor modulator or other pain-relieving agent. Side effects of 5-HT_{1A} receptor modulators that the selected combinations of the present invention may reduce or avoid are nausea, dizziness, insomnia, headache, fatigue, paresthesias, uneasiness, nervousness, lightheadedness, excitement, tachycardia, malaise, dysphoria, dry mouth, headache, somnolence, constipation, abnormal movements, respiratory disorders, dyspepsia, skin rash, elevations in liver enzymes and gastrointestinal disturbances.

The phrase “adjunctive therapy” encompasses treatment of a subject with agents that reduce or avoid side effects associated with the combination therapy of the present invention.

Dosages, Formulations and Routes of Administration:

Dosage levels of the Cox-2 inhibiting agent (e.g., a Cox-2 selective inhibiting agent or a prodrug of a Cox-2 selective inhibiting agent) on the order of about 0.1 mg to about 10,000 mg of the active ingredient compound are useful in the treatment of the above conditions, with preferred levels of about 1.0 mg to about 1,000 mg. While the dosage of active compound administered to a warm-blooded animal (a mammal), is dependent on the species of that mammal, the body weight, age, and individual condition, and on the route of administration, the unit dosage for oral administration to a mammal of about 50 to 70 kg may contain between about 5 and 500 mg of the active ingredient (for example, Cox-189). The amount of active ingredient that may be combined with a 5-HT_{1A} receptor modulator to produce a single dosage form will vary depending upon the host treated and the particular mode of administration.

A total daily dose of a 5-HT_{1A} receptor modulator can generally be in the range of from about 0.001 to about 10,000 mg/day in single or divided doses, with preferred levels of about 1.0 mg to about 1,000 mg. It is understood, however, that specific dose levels of the therapeutic agents or therapeutic approaches of the present invention for any particular subject depends upon a variety of factors including the activity of the specific compound employed, the age, body weight, general health, sex, and diet of the subject, the time of administration, the rate of excretion, the drug combination, and the severity of the particular disease being treated and form of administration.

Treatment dosages generally may be titrated to optimize safety and efficacy. Typically, dosage-effect relationships from in vitro initially can provide useful guidance on the proper doses for subject administration. Studies in animal models also generally may be used for guidance regarding effective dosages for treatment of pain, inflammation, or an inflammation-related disorder in accordance with the present invention. In terms of treatment protocols, it should be appreciated that the dosage to be administered will depend on several factors, including the particular agent that is administered, the route administered, the condition of the particular subject, etc. Generally speaking, one will desire to administer an amount of the compound that is effective to achieve a serum level commensurate with the concentrations found to be effective in vitro. Thus, where a compound is found to demonstrate in vitro activity at, e.g., 10 μM, one will desire to administer an amount of the drug that is effective to provide about a 10 μM concentration in vivo. Determination of these parameters is well within the skill of the art.

Effective formulations and administration procedures are well known in the art and are described in standard textbooks.

The Cox-2 inhibiting agents or the 5-HT_{1A} receptor modulators can be formulated as a single pharmaceutical composition or as independent multiple pharmaceutical compositions. Pharmaceutical compositions according to the present invention include those suitable for oral, inhalation spray, rectal, topical, buccal (e.g., sublingual), or parenteral (e.g., subcutaneous, intramuscular, intravenous, intrathecal, intramedullary and intradermal injections, or infusion techniques) administration, although the most suitable route in any given case will depend on the nature and severity of the condition being treated and on the nature of the particular compound which is being used. In most cases, the preferred route of administration is oral or parenteral.

Compounds and composition of the present invention can then be administered orally, by inhalation spray, rectally, topically, buccally or parenterally in dosage unit formulations containing conventional nontoxic pharmaceutically acceptable carriers, adjuvants, and vehicles as desired. The compounds of the present invention can be administered by any conventional means available for use in conjunction with pharmaceuticals, either as individual therapeutic compounds or as a combination of therapeutic compounds.

Pharmaceutically acceptable salts are particularly suitable for medical applications because of their greater aqueous solubility relative to the parent compound. Such salts must clearly have a pharmaceutically acceptable anion or cation.

The compounds useful in the methods, combinations and compositions of the present invention can be presented with an acceptable carrier in the form of a pharmaceutical composition. The carrier must, of course, be acceptable in the sense of being compatible with the other ingredients of the composition and must not be deleterious to the recipient. The carrier can be a solid or a liquid, or both, and is preferably formulated with the compound as a unit-dose composition, for example, a tablet, which can contain from 0.05% to 95% by weight of the active compound. Other pharmacologically active substances can also be present, including other compounds of the present invention. The pharmaceutical compositions of the invention can be prepared by any of the well-known techniques of pharmacy, consisting essentially of admixing the components.

The amount of compound in combination that is required to achieve the desired biological effect will, of course, depend on a number of factors such as the specific compound chosen, the use for which it is intended, the mode of administration, and the clinical condition of the recipient.

The compounds of the present invention can be delivered orally either in a solid, in a semi-solid, or in a liquid form. Dosing for oral administration may be with a regimen calling for single daily dose, or for a single dose every other day, or for multiple, spaced doses throughout the day. For oral administration, the pharmaceutical composition may be in the form of, for example, a tablet, capsule, suspension, or liquid. Capsules, tablets, etc., can be prepared by conventional methods well known in the art. The pharmaceutical composition is preferably made in the form of a dosage unit containing a particular amount of the active ingredient or ingredients. Examples of dosage units are tablets or capsules, and may contain one or more therapeutic compounds in an amount described herein. For example, in the case of a 5-HT_{1A} receptor modulator, the dose range may be from about 0.01 mg to about 5,000 mg or any other dose, dependent upon the specific inhibitor, as is known in the art. When in a liquid or in a semi-solid form, the combinations of the present invention can, for example, be in the form of a liquid, syrup, or contained in a gel capsule (e.g., a gel cap). In one embodiment, when a 5-HT_{1A} receptor modulator is used in a combination of the present invention, the 5-HT_{1A} receptor modulator can be provided in the form of a liquid, syrup, or contained in a gel capsule. In another embodiment, when a Cox-2 inhibiting agent is used in a combination of the present invention, the Cox-2 inhibiting agent can be provided in the form of a liquid, syrup, or contained in a gel capsule.

Oral delivery of the combinations of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. For some of the therapeutic compounds useful in the methods, combinations and compositions of the present invention the intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Pharmaceutical compositions suitable for oral administration can be presented in discrete units, such as capsules, cachets, lozenges, or tablets, each containing a predetermined amount of at least one therapeutic compound useful in the present invention; as a powder or granules; as a solution or a suspension in an aqueous or non-aqueous liquid; or as an oil-in-water or water-in-oil emulsion. As indicated, such compositions can be prepared by any suitable method of pharmacy which includes the step of bringing into association the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more assessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Pharmaceutical compositions suitable for buccal (sub-lingual) administration include lozenges comprising a compound of the present invention in a flavored base, usually sucrose, and acacia or tragacanth, and pastilles comprising the compound in an inert base such as gelatin and glycerin or sucrose and acacia.

Pharmaceutical compositions suitable for parenteral administration conveniently comprise sterile aqueous preparations of a compound of the present invention. These preparations are preferably administered intravenously, although administration can also be effected by means of subcutaneous, intramuscular, or intradermal injection or by infusion. Such preparations can conveniently be prepared by admixing the compound with water and rendering the resulting solution sterile and isotonic with the blood. Injectable compositions according to the invention will generally contain from 0.1 to 10% w/w of a compound disclosed herein.

Injectable preparations, for example, sterile injectable aqueous or oleaginous suspensions may be formulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The active ingredients may also be administered by injection as a composition wherein, for example, saline, dextrose, or water may be used as a suitable carrier. A suitable daily dose of each active therapeutic compound is one that achieves the same blood serum level as produced by oral administration as described above.

The dose of any of these therapeutic compounds can be conveniently administered as an infusion of from about 10 ng/kg body weight to about 10,000 ng/kg body weight per minute. Infusion fluids suitable for this purpose can contain, for example, from about 0.1 ng to about 10 mg, preferably from about 1 ng to about 10 mg per milliliter. Unit doses can contain, for example, from about 1 mg to about 10 g of the compound of the present invention. Thus, ampules for injection can contain, for example, from about 1 mg to about 100 mg.

Pharmaceutical compositions suitable for rectal administration are preferably presented as unit-dose suppositories. These can be prepared by admixing a compound or compounds of the present invention with one or more conventional solid carriers, for example, cocoa butter, synthetic mono- di- or triglycerides, fatty acids and polyethylene glycols that are solid at ordinary temperatures but liquid at the rectal temperature and will therefore melt in the rectum and release the drug; and then shaping the resulting mixture.

Pharmaceutical compositions suitable for topical application to the skin preferably take the form of an ointment, cream, lotion, paste, gel, spray, aerosol, or oil. Carriers which can be used include petroleum jelly (e.g., Vaseline), lanolin, polyethylene glycols, alcohols, and combinations of two or more thereof. The active compound or compounds are generally present at a concentration of from 0.1 to 50% w/w of the composition, for example, from 0.5 to 2%.

Transdermal administration is also possible. Pharmaceutical compositions suitable for transdermal administration can be presented as discrete patches adapted to remain in intimate contact with the epidermis of the recipient for a prolonged period of time. Such patches suitably contain a compound or compounds of the present invention in an optionally buffered, aqueous solution, dissolved and/or dispersed in an adhesive, or dispersed in a polymer. A suitable concentration of the active compound or compounds is about 1% to 35%, preferably about 3% to 15%. As one particular possibility, the compound or compounds can be delivered from the patch by electrotransport or iontophoresis, for example, as described in Pharmaceutical Research, 3(6), 318 (1986).

In any case, the amount of active ingredients that can be combined with carrier materials to produce a single dosage form to be administered will vary depending upon the host treated and the particular mode of administration.

In combination therapy, administration of two or more of the therapeutic agents useful in the methods, combinations and compositions of the present invention may take place sequentially in separate formulations, or may be accomplished by simultaneous administration in a single formulation or in a separate formulation. Independent administration of each therapeutic agent may be accomplished by, for example, oral, inhalation spray, rectal, topical, buccal (e.g., sublingual), or parenteral (e.g., subcutaneous, intramuscular, intravenous, intrathecal, intramedullary and intradermal injections, or infusion techniques) administration. The formulation may be in the form of a bolus, or in the form of aqueous or non-aqueous isotonic sterile injection solutions or suspensions. Solutions and suspensions may be prepared from sterile powders or granules having one or more pharmaceutically-acceptable carriers or diluents, or a binder such as gelatin or hydroxypropylmethyl cellulose, together with one or more of a lubricant, preservative, surface active or dispersing agent. The therapeutic compounds may further be administered by any combination of, for example, oral/oral, oral/parenteral, or parenteral/parenteral route.

The therapeutic compounds which make up the combination therapy may be a combined dosage form or in separate dosage forms intended for substantially simultaneous oral administration. The therapeutic compounds which make up the combination therapy may also be administered sequentially, with either therapeutic compound being administered by a regimen calling for two step ingestion. Thus, a regimen may call for sequential administration of the therapeutic compounds with spaced-apart ingestion of the separate, active agents. The time period between the multiple ingestion steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by intravenous route. Whether the therapeutic compounds of the combined therapy are administered orally, by inhalation spray, rectally, topically, buccally (e.g., sublingual), or parenterally (e.g., subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components. Examples of suitable pharmaceutically-acceptable formulations containing the therapeutic compounds are given above. Additionally, drug formulations are discussed in, for example, Hoover, John E., Remington's Pharmaceutical Sciences, Mack Publishing Co., Easton, Pa. 1975. Another discussion of drug formulations can be found in Liberman, H. A. and Lachman, L., Eds., Pharmaceutical Dosage Forms, Marcel Decker, New York, N.Y., 1980.

The following examples describe preferred embodiments of the invention. Other embodiments within the scope of the claims herein will be apparent to one skilled in the art from consideration of the specification or practice of the invention as disclosed herein. It is intended that the specification, taken together with the examples, be considered to be exemplary only, with the scope and spirit of the invention being indicated by the claims which follow the examples. In the examples, all percentages are given on a weight basis, unless otherwise indicated.

### EXAMPLE 1

This example illustrates combinations of the present invention.

Table 5 describes a number of combinations comprising a Cox-2 selective inhibitor and a 5-HT_{1A} receptor modulator. Designations of “H” corresponds to compounds described above in the specification.

TABLE 5
Combinations of Cox-2 selective inhibiting agents
and 5-HT_{1A} receptor modulators.

| Example | | 5-HT_{1A} Receptor |
|---|---|---|
| Number | Cox-2 Inhibitor | Modulator |
| 1 | Celecoxib | H1 |
| 2 | Celecoxib | H2 |
| 3 | Celecoxib | H3 |
| 4 | Celecoxib | H4 |
| 5 | Celecoxib | H5 |
| 6 | Celecoxib | H6 |
| 7 | Celecoxib | H7 |
| 8 | Celecoxib | H8 |
| 9 | Celecoxib | H9 |
| 10 | Celecoxib | H10 |
| 11 | Celecoxib | H11 |
| 12 | Celecoxib | H12 |
| 13 | Celecoxib | H13 |
| 14 | Celecoxib | H14 |
| 15 | Celecoxib | H15 |
| 16 | Celecoxib | H16 |
| 17 | Celecoxib | H17 |
| 18 | Celecoxib | H18 |
| 19 | Celecoxib | H19 |
| 20 | Celecoxib | H20 |
| 21 | Celecoxib | H21 |
| 22 | Celecoxib | H22 |
| 23 | Celecoxib | H23 |
| 24 | Celecoxib | H24 |
| 25 | Celecoxib | H25 |
| 26 | Celecoxib | H26 |
| 27 | Celecoxib | H27 |
| 28 | Celecoxib | H28 |
| 29 | Celecoxib | H29 |
| 30 | Celecoxib | H30 |
| 31 | Celecoxib | H31 |
| 32 | Celecoxib | H32 |
| 33 | Celecoxib | H33 |
| 34 | Celecoxib | H34 |
| 35 | Celecoxib | H35 |
| 36 | Celecoxib | H36 |
| 37 | Celecoxib | H37 |
| 38 | Celecoxib | H38 |
| 39 | Celecoxib | H39 |
| 40 | Celecoxib | H40 |
| 41 | Celecoxib | H41 |
| 42 | Celecoxib | H42 |
| 43 | Celecoxib | H43 |
| 44 | Celecoxib | H44 |
| 45 | Celecoxib | H45 |
| 46 | Celecoxib | H46 |
| 47 | Celecoxib | H47 |
| 48 | Celecoxib | H48 |
| 49 | Celecoxib | H49 |
| 50 | Celecoxib | H50 |
| 51 | Celecoxib | H51 |
| 52 | Celecoxib | H52 |
| 53 | Celecoxib | H53 |
| 54 | Celecoxib | H54 |
| 55 | Celecoxib | H55 |
| 56 | Celecoxib | H56 |
| 57 | Celecoxib | H57 |
| 58 | Celecoxib | H58 |
| 59 | Celecoxib | H59 |
| 60 | Celecoxib | H60 |
| 61 | Celecoxib | H61 |
| 62 | Celecoxib | H62 |
| 63 | Celecoxib | H63 |
| 64 | Celecoxib | H64 |
| 65 | Celecoxib | H65 |
| 66 | Celecoxib | H66 |
| 67 | Celecoxib | H67 |
| 68 | Celecoxib | H68 |
| 69 | Celecoxib | H69 |
| 70 | Celecoxib | H70 |
| 71 | Celecoxib | H71 |
| 72 | Celecoxib | H72 |
| 73 | Celecoxib | H73 |
| 74 | Celecoxib | H74 |
| 75 | Celecoxib | H75 |
| 76 | Celecoxib | H76 |
| 77 | Celecoxib | H77 |
| 78 | Celecoxib | H78 |
| 79 | Celecoxib | H79 |
| 80 | Celecoxib | H80 |
| 81 | Celecoxib | H81 |
| 82 | Celecoxib | H82 |
| 83 | Celecoxib | H83 |
| 84 | Celecoxib | H84 |
| 85 | Valdecoxib | H1 |
| 86 | Valdecoxib | H2 |
| 87 | Valdecoxib | H3 |
| 88 | Valdecoxib | H4 |
| 89 | Valdecoxib | H5 |
| 90 | Valdecoxib | H6 |
| 91 | Valdecoxib | H7 |
| 92 | Valdecoxib | H8 |
| 93 | Valdecoxib | H9 |
| 94 | Valdecoxib | H10 |
| 95 | Valdecoxib | H11 |
| 96 | Valdecoxib | H12 |
| 97 | Valdecoxib | H13 |
| 98 | Valdecoxib | H14 |
| 99 | Valdecoxib | H15 |
| 100 | Valdecoxib | H16 |
| 101 | Valdecoxib | H17 |
| 102 | Valdecoxib | H18 |
| 103 | Valdecoxib | H19 |
| 104 | Valdecoxib | H20 |
| 105 | Valdecoxib | H21 |
| 106 | Valdecoxib | H22 |
| 107 | Valdecoxib | H23 |
| 108 | Valdecoxib | H24 |
| 109 | Valdecoxib | H25 |
| 110 | Valdecoxib | H26 |
| 111 | Valdecoxib | H27 |
| 112 | Valdecoxib | H28 |
| 113 | Valdecoxib | H29 |
| 114 | Valdecoxib | H30 |
| 115 | Valdecoxib | H31 |
| 116 | Valdecoxib | H32 |
| 117 | Valdecoxib | H33 |
| 118 | Valdecoxib | H34 |
| 119 | Valdecoxib | H35 |
| 120 | Valdecoxib | H36 |
| 121 | Valdecoxib | H37 |
| 122 | Valdecoxib | H38 |
| 123 | Valdecoxib | H39 |
| 124 | Valdecoxib | H40 |
| 125 | Valdecoxib | H41 |
| 126 | Valdecoxib | H42 |
| 127 | Valdecoxib | H43 |
| 128 | Valdecoxib | H44 |
| 129 | Valdecoxib | H45 |
| 130 | Valdecoxib | H46 |
| 131 | Valdecoxib | H47 |
| 132 | Valdecoxib | H48 |
| 133 | Valdecoxib | H49 |
| 134 | Valdecoxib | H50 |
| 135 | Valdecoxib | H51 |
| 136 | Valdecoxib | H52 |
| 137 | Valdecoxib | H53 |
| 138 | Valdecoxib | H54 |
| 139 | Valdecoxib | H55 |
| 140 | Valdecoxib | H56 |
| 141 | Valdecoxib | H57 |
| 142 | Valdecoxib | H58 |
| 143 | Valdecoxib | H59 |
| 144 | Valdecoxib | H60 |
| 145 | Valdecoxib | H61 |
| 146 | Valdecoxib | H62 |
| 147 | Valdecoxib | H63 |
| 148 | Valdecoxib | H64 |
| 149 | Valdecoxib | H65 |
| 150 | Valdecoxib | H66 |
| 151 | Valdecoxib | H67 |
| 152 | Valdecoxib | H68 |
| 153 | Valdecoxib | H69 |
| 154 | Valdecoxib | H70 |
| 155 | Valdecoxib | H71 |
| 156 | Valdecoxib | H72 |
| 157 | Valdecoxib | H73 |
| 158 | Valdecoxib | H74 |
| 159 | Valdecoxib | H75 |
| 160 | Valdecoxib | H76 |
| 161 | Valdecoxib | H77 |
| 162 | Valdecoxib | H78 |
| 163 | Valdecoxib | H79 |
| 164 | Valdecoxib | H80 |
| 165 | Valdecoxib | H81 |
| 166 | Valdecoxib | H82 |
| 167 | Valdecoxib | H83 |
| 168 | Valdecoxib | H84 |
| 169 | Parecoxib | H1 |
| 170 | Parecoxib | H2 |
| 171 | Parecoxib | H3 |
| 172 | Parecoxib | H4 |
| 173 | Parecoxib | H5 |
| 174 | Parecoxib | H6 |
| 175 | Parecoxib | H7 |
| 176 | Parecoxib | H8 |
| 177 | Parecoxib | H9 |
| 178 | Parecoxib | H10 |
| 179 | Parecoxib | H11 |
| 180 | Parecoxib | H12 |
| 181 | Parecoxib | H13 |
| 182 | Parecoxib | H14 |
| 183 | Parecoxib | H15 |
| 184 | Parecoxib | H16 |
| 185 | Parecoxib | H17 |
| 186 | Parecoxib | H18 |
| 187 | Parecoxib | H19 |
| 188 | Parecoxib | H20 |
| 189 | Parecoxib | H21 |
| 190 | Parecoxib | H22 |
| 191 | Parecoxib | H23 |
| 192 | Parecoxib | H24 |
| 193 | Parecoxib | H25 |
| 194 | Parecoxib | H26 |
| 195 | Parecoxib | H27 |
| 196 | Parecoxib | H28 |
| 197 | Parecoxib | H29 |
| 198 | Parecoxib | H30 |
| 199 | Parecoxib | H31 |
| 200 | Parecoxib | H32 |
| 201 | Parecoxib | H33 |
| 202 | Parecoxib | H34 |
| 203 | Parecoxib | H35 |
| 204 | Parecoxib | H36 |
| 205 | Parecoxib | H37 |
| 206 | Parecoxib | H38 |
| 207 | Parecoxib | H39 |
| 208 | Parecoxib | H40 |
| 209 | Parecoxib | H41 |
| 210 | Parecoxib | H42 |
| 211 | Parecoxib | H43 |
| 212 | Parecoxib | H44 |
| 213 | Parecoxib | H45 |
| 214 | Parecoxib | H46 |
| 215 | Parecoxib | H47 |
| 216 | Parecoxib | H48 |
| 217 | Parecoxib | H49 |
| 218 | Parecoxib | H50 |
| 219 | Parecoxib | H51 |
| 220 | Parecoxib | H52 |
| 221 | Parecoxib | H53 |
| 222 | Parecoxib | H54 |
| 223 | Parecoxib | H55 |
| 224 | Parecoxib | H56 |
| 225 | Parecoxib | H57 |
| 226 | Parecoxib | H58 |
| 227 | Parecoxib | H59 |
| 228 | Parecoxib | H60 |
| 229 | Parecoxib | H61 |
| 230 | Parecoxib | H62 |
| 231 | Parecoxib | H63 |
| 232 | Parecoxib | H64 |
| 233 | Parecoxib | H65 |
| 234 | Parecoxib | H66 |
| 235 | Parecoxib | H67 |
| 236 | Parecoxib | H68 |
| 237 | Parecoxib | H69 |
| 238 | Parecoxib | H70 |
| 239 | Parecoxib | H71 |
| 240 | Parecoxib | H72 |
| 241 | Parecoxib | H73 |
| 242 | Parecoxib | H74 |
| 243 | Parecoxib | H75 |
| 244 | Parecoxib | H76 |
| 245 | Parecoxib | H77 |
| 246 | Parecoxib | H78 |
| 247 | Parecoxib | H79 |
| 248 | Parecoxib | H80 |
| 249 | Parecoxib | H81 |
| 250 | Parecoxib | H82 |
| 251 | Parecoxib | H83 |
| 252 | Parecoxib | H84 |
| 253 | Deracoxib | H1 |
| 254 | Deracoxib | H2 |
| 255 | Deracoxib | H3 |
| 256 | Deracoxib | H4 |
| 257 | Deracoxib | H5 |
| 258 | Deracoxib | H6 |
| 259 | Deracoxib | H7 |
| 260 | Deracoxib | H8 |
| 261 | Deracoxib | H9 |
| 262 | Deracoxib | H10 |
| 263 | Deracoxib | H11 |
| 264 | Deracoxib | H12 |
| 265 | Deracoxib | H13 |
| 266 | Deracoxib | H14 |
| 267 | Deracoxib | H15 |
| 268 | Deracoxib | H16 |
| 269 | Deracoxib | H17 |
| 270 | Deracoxib | H18 |
| 271 | Deracoxib | H19 |
| 272 | Deracoxib | H20 |
| 273 | Deracoxib | H21 |
| 274 | Deracoxib | H22 |
| 275 | Deracoxib | H23 |
| 276 | Deracoxib | H24 |
| 277 | Deracoxib | H25 |
| 278 | Deracoxib | H26 |
| 279 | Deracoxib | H27 |
| 280 | Deracoxib | H28 |
| 281 | Deracoxib | H29 |
| 282 | Deracoxib | H30 |
| 283 | Deracoxib | H31 |
| 284 | Deracoxib | H32 |
| 285 | Deracoxib | H33 |
| 286 | Deracoxib | H34 |
| 287 | Deracoxib | H35 |
| 288 | Deracoxib | H36 |
| 289 | Deracoxib | H37 |
| 290 | Deracoxib | H38 |
| 291 | Deracoxib | H39 |
| 292 | Deracoxib | H40 |
| 293 | Deracoxib | H41 |
| 294 | Deracoxib | H42 |
| 295 | Deracoxib | H43 |
| 296 | Deracoxib | H44 |
| 297 | Deracoxib | H45 |
| 298 | Deracoxib | H46 |
| 299 | Deracoxib | H47 |
| 300 | Deracoxib | H48 |
| 301 | Deracoxib | H49 |
| 302 | Deracoxib | H50 |
| 303 | Deracoxib | H51 |
| 304 | Deracoxib | H52 |
| 305 | Deracoxib | H53 |
| 306 | Deracoxib | H54 |
| 307 | Deracoxib | H55 |
| 308 | Deracoxib | H56 |
| 309 | Deracoxib | H57 |
| 310 | Deracoxib | H58 |
| 311 | Deracoxib | H59 |
| 312 | Deracoxib | H60 |
| 313 | Deracoxib | H61 |
| 314 | Deracoxib | H62 |
| 315 | Deracoxib | H63 |
| 316 | Deracoxib | H64 |
| 317 | Deracoxib | H65 |
| 318 | Deracoxib | H66 |
| 319 | Deracoxib | H67 |
| 320 | Deracoxib | H68 |
| 321 | Deracoxib | H69 |
| 322 | Deracoxib | H70 |
| 323 | Deracoxib | H71 |
| 324 | Deracoxib | H72 |
| 325 | Deracoxib | H73 |
| 326 | Deracoxib | H74 |
| 327 | Deracoxib | H75 |
| 328 | Deracoxib | H76 |
| 329 | Deracoxib | H77 |
| 330 | Deracoxib | H78 |
| 331 | Deracoxib | H79 |
| 332 | Deracoxib | H80 |
| 333 | Deracoxib | H81 |
| 334 | Deracoxib | H82 |
| 335 | Deracoxib | H83 |
| 336 | Deracoxib | H84 |
| 337 | Etorixocib | H1 |
| 338 | Etorixocib | H2 |
| 339 | Etorixocib | H3 |
| 340 | Etorixocib | H4 |
| 341 | Etorixocib | H5 |
| 342 | Etorixocib | H6 |
| 343 | Etorixocib | H7 |
| 344 | Etorixocib | H8 |
| 345 | Etorixocib | H9 |
| 346 | Etorixocib | H10 |
| 347 | Etorixocib | H11 |
| 348 | Etorixocib | H12 |
| 349 | Etorixocib | H13 |
| 350 | Etorixocib | H14 |
| 351 | Etorixocib | H15 |
| 352 | Etorixocib | H16 |
| 353 | Etorixocib | H17 |
| 354 | Etorixocib | H18 |
| 355 | Etorixocib | H19 |
| 356 | Etorixocib | H20 |
| 357 | Etorixocib | H21 |
| 358 | Etorixocib | H22 |
| 359 | Etorixocib | H23 |
| 360 | Etorixocib | H24 |
| 361 | Etorixocib | H25 |
| 362 | Etorixocib | H26 |
| 363 | Etorixocib | H27 |
| 364 | Etorixocib | H28 |
| 365 | Etorixocib | H29 |
| 366 | Etorixocib | H30 |
| 367 | Etorixocib | H31 |
| 368 | Etorixocib | H32 |
| 369 | Etorixocib | H33 |
| 370 | Etorixocib | H34 |
| 371 | Etorixocib | H35 |
| 372 | Etorixocib | H36 |
| 373 | Etorixocib | H37 |
| 374 | Etorixocib | H38 |
| 375 | Etorixocib | H39 |
| 376 | Etorixocib | H40 |
| 377 | Etorixocib | H41 |
| 378 | Etorixocib | H42 |
| 379 | Etorixocib | H43 |
| 380 | Etorixocib | H44 |
| 381 | Etorixocib | H45 |
| 382 | Etorixocib | H46 |
| 383 | Etorixocib | H47 |
| 384 | Etorixocib | H48 |
| 385 | Etorixocib | H49 |
| 386 | Etorixocib | H50 |
| 387 | Etorixocib | H51 |
| 388 | Etorixocib | H52 |
| 389 | Etorixocib | H53 |
| 390 | Etorixocib | H54 |
| 391 | Etorixocib | H55 |
| 392 | Etorixocib | H56 |
| 393 | Etorixocib | H57 |
| 394 | Etorixocib | H58 |
| 395 | Etorixocib | H59 |
| 396 | Etorixocib | H60 |
| 397 | Etorixocib | H61 |
| 398 | Etorixocib | H62 |
| 399 | Etorixocib | H63 |
| 400 | Etorixocib | H64 |
| 401 | Etorixocib | H65 |
| 402 | Etorixocib | H66 |
| 403 | Etorixocib | H67 |
| 404 | Etorixocib | H68 |
| 405 | Etorixocib | H69 |
| 406 | Etorixocib | H70 |
| 407 | Etorixocib | H71 |
| 408 | Etorixocib | H72 |
| 409 | Etorixocib | H73 |
| 410 | Etorixocib | H74 |
| 411 | Etorixocib | H75 |
| 412 | Etorixocib | H76 |
| 413 | Etorixocib | H77 |
| 414 | Etorixocib | H78 |
| 415 | Etorixocib | H79 |
| 416 | Etorixocib | H80 |
| 417 | Etorixocib | H81 |
| 418 | Etorixocib | H82 |
| 419 | Etorixocib | H83 |
| 420 | Etorixocib | H84 |
| 421 | Lumiracoxib | H1 |
| 422 | Lumiracoxib | H2 |
| 423 | Lumiracoxib | H3 |
| 424 | Lumiracoxib | H4 |
| 425 | Lumiracoxib | H5 |
| 426 | Lumiracoxib | H6 |
| 427 | Lumiracoxib | H7 |
| 428 | Lumiracoxib | H8 |
| 429 | Lumiracoxib | H9 |
| 430 | Lumiracoxib | H10 |
| 431 | Lumiracoxib | H11 |
| 432 | Lumiracoxib | H12 |
| 433 | Lumiracoxib | H13 |
| 434 | Lumiracoxib | H14 |
| 435 | Lumiracoxib | H15 |
| 436 | Lumiracoxib | H16 |
| 437 | Lumiracoxib | H17 |
| 438 | Lumiracoxib | H18 |
| 439 | Lumiracoxib | H19 |
| 440 | Lumiracoxib | H20 |
| 441 | Lumiracoxib | H21 |
| 442 | Lumiracoxib | H22 |
| 443 | Lumiracoxib | H23 |
| 444 | Lumiracoxib | H24 |
| 445 | Lumiracoxib | H25 |
| 446 | Lumiracoxib | H26 |
| 447 | Lumiracoxib | H27 |
| 448 | Lumiracoxib | H28 |
| 449 | Lumiracoxib | H29 |
| 450 | Lumiracoxib | H30 |
| 451 | Lumiracoxib | H31 |
| 452 | Lumiracoxib | H32 |
| 453 | Lumiracoxib | H33 |
| 454 | Lumiracoxib | H34 |
| 455 | Lumiracoxib | H35 |
| 456 | Lumiracoxib | H36 |
| 457 | Lumiracoxib | H37 |
| 458 | Lumiracoxib | H38 |
| 459 | Lumiracoxib | H39 |
| 460 | Lumiracoxib | H40 |
| 461 | Lumiracoxib | H41 |
| 462 | Lumiracoxib | H42 |
| 463 | Lumiracoxib | H43 |
| 464 | Lumiracoxib | H44 |
| 465 | Lumiracoxib | H45 |
| 466 | Lumiracoxib | H46 |
| 467 | Lumiracoxib | H47 |
| 468 | Lumiracoxib | H48 |
| 469 | Lumiracoxib | H49 |
| 470 | Lumiracoxib | H50 |
| 471 | Lumiracoxib | H51 |
| 472 | Lumiracoxib | H52 |
| 473 | Lumiracoxib | H53 |
| 474 | Lumiracoxib | H54 |
| 475 | Lumiracoxib | H55 |
| 476 | Lumiracoxib | H56 |
| 477 | Lumiracoxib | H57 |
| 478 | Lumiracoxib | H58 |
| 479 | Lumiracoxib | H59 |
| 480 | Lumiracoxib | H60 |
| 481 | Lumiracoxib | H61 |
| 482 | Lumiracoxib | H62 |
| 483 | Lumiracoxib | H63 |
| 484 | Lumiracoxib | H64 |
| 485 | Lumiracoxib | H65 |
| 486 | Lumiracoxib | H66 |
| 487 | Lumiracoxib | H67 |
| 488 | Lumiracoxib | H68 |
| 489 | Lumiracoxib | H69 |
| 490 | Lumiracoxib | H70 |
| 491 | Lumiracoxib | H71 |
| 492 | Lumiracoxib | H72 |
| 493 | Lumiracoxib | H73 |
| 494 | Lumiracoxib | H74 |
| 495 | Lumiracoxib | H75 |
| 496 | Lumiracoxib | H76 |
| 497 | Lumiracoxib | H77 |
| 498 | Lumiracoxib | H78 |
| 499 | Lumiracoxib | H79 |
| 500 | Lumiracoxib | H80 |
| 501 | Lumiracoxib | H81 |
| 502 | Lumiracoxib | H82 |
| 503 | Lumiracoxib | H83 |
| 504 | Lumiracoxib | H84 |
| 505 | Deracoxib | H1 |
| 506 | Deracoxib | H2 |
| 507 | Deracoxib | H3 |
| 508 | Deracoxib | H4 |
| 509 | Deracoxib | H5 |
| 510 | Deracoxib | H6 |
| 511 | Deracoxib | H7 |
| 512 | Deracoxib | H8 |
| 513 | Deracoxib | H9 |
| 514 | Deracoxib | H10 |
| 515 | Deracoxib | H11 |
| 516 | Deracoxib | H12 |
| 517 | Deracoxib | H13 |
| 518 | Deracoxib | H14 |
| 519 | Deracoxib | H15 |
| 520 | Deracoxib | H16 |
| 521 | Deracoxib | H17 |
| 522 | Deracoxib | H18 |
| 523 | Deracoxib | H19 |
| 524 | Deracoxib | H20 |
| 525 | Deracoxib | H21 |
| 526 | Deracoxib | H22 |
| 527 | Deracoxib | H23 |
| 528 | Deracoxib | H24 |
| 529 | Deracoxib | H25 |
| 530 | Deracoxib | H26 |
| 531 | Deracoxib | H27 |
| 532 | Deracoxib | H28 |
| 533 | Deracoxib | H29 |
| 534 | Deracoxib | H30 |
| 535 | Deracoxib | H31 |
| 536 | Deracoxib | H32 |
| 537 | Deracoxib | H33 |
| 538 | Deracoxib | H34 |
| 539 | Deracoxib | H35 |
| 540 | Deracoxib | H36 |
| 541 | Deracoxib | H37 |
| 542 | Deracoxib | H38 |
| 543 | Deracoxib | H39 |
| 544 | Deracoxib | H40 |
| 545 | Deracoxib | H41 |
| 546 | Deracoxib | H42 |
| 547 | Deracoxib | H43 |
| 548 | Deracoxib | H44 |
| 549 | Deracoxib | H45 |
| 550 | Deracoxib | H46 |
| 551 | Deracoxib | H47 |
| 552 | Deracoxib | H48 |
| 553 | Deracoxib | H49 |
| 554 | Deracoxib | H50 |
| 555 | Deracoxib | H51 |
| 556 | Deracoxib | H52 |
| 557 | Deracoxib | H53 |
| 558 | Deracoxib | H54 |
| 559 | Deracoxib | H55 |
| 560 | Deracoxib | H56 |
| 561 | Deracoxib | H57 |
| 562 | Deracoxib | H58 |
| 563 | Deracoxib | H59 |
| 564 | Deracoxib | H60 |
| 565 | Deracoxib | H61 |
| 566 | Deracoxib | H62 |
| 567 | Deracoxib | H63 |
| 568 | Deracoxib | H64 |
| 569 | Deracoxib | H65 |
| 570 | Deracoxib | H66 |
| 571 | Deracoxib | H67 |
| 572 | Deracoxib | H68 |
| 573 | Deracoxib | H69 |
| 574 | Deracoxib | H70 |
| 575 | Deracoxib | H71 |
| 576 | Deracoxib | H72 |
| 577 | Deracoxib | H73 |
| 578 | Deracoxib | H74 |
| 579 | Deracoxib | H75 |
| 580 | Deracoxib | H76 |
| 581 | Deracoxib | H77 |
| 582 | Deracoxib | H78 |
| 583 | Deracoxib | H79 |
| 584 | Deracoxib | H80 |
| 585 | Deracoxib | H81 |
| 586 | Deracoxib | H82 |
| 587 | Deracoxib | H83 |
| 588 | Deracoxib | H84 |
| 589 | Rofecoxib | H1 |
| 590 | Rofecoxib | H2 |
| 591 | Rofecoxib | H3 |
| 592 | Rofecoxib | H4 |
| 593 | Rofecoxib | H5 |
| 594 | Rofecoxib | H6 |
| 595 | Rofecoxib | H7 |
| 596 | Rofecoxib | H8 |
| 597 | Rofecoxib | H9 |
| 598 | Rofecoxib | H10 |
| 599 | Rofecoxib | H11 |
| 600 | Rofecoxib | H12 |
| 601 | Rofecoxib | H13 |
| 602 | Rofecoxib | H14 |
| 603 | Rofecoxib | H15 |
| 604 | Rofecoxib | H16 |
| 605 | Rofecoxib | H17 |
| 606 | Rofecoxib | H18 |
| 607 | Rofecoxib | H19 |
| 608 | Rofecoxib | H20 |
| 609 | Rofecoxib | H21 |
| 610 | Rofecoxib | H22 |
| 611 | Rofecoxib | H23 |
| 612 | Rofecoxib | H24 |
| 613 | Rofecoxib | H25 |
| 614 | Rofecoxib | H26 |
| 615 | Rofecoxib | H27 |
| 616 | Rofecoxib | H28 |
| 617 | Rofecoxib | H29 |
| 618 | Rofecoxib | H30 |
| 619 | Rofecoxib | H31 |
| 620 | Rofecoxib | H32 |
| 621 | Rofecoxib | H33 |
| 622 | Rofecoxib | H34 |
| 623 | Rofecoxib | H35 |
| 624 | Rofecoxib | H36 |
| 625 | Rofecoxib | H37 |
| 626 | Rofecoxib | H38 |
| 627 | Rofecoxib | H39 |
| 628 | Rofecoxib | H40 |
| 629 | Rofecoxib | H41 |
| 630 | Rofecoxib | H42 |
| 631 | Rofecoxib | H43 |
| 632 | Rofecoxib | H44 |
| 633 | Rofecoxib | H45 |
| 634 | Rofecoxib | H46 |
| 635 | Rofecoxib | H47 |
| 636 | Rofecoxib | H48 |
| 637 | Rofecoxib | H49 |
| 638 | Rofecoxib | H50 |
| 639 | Rofecoxib | H51 |
| 640 | Rofecoxib | H52 |
| 641 | Rofecoxib | H53 |
| 642 | Rofecoxib | H54 |
| 643 | Rofecoxib | H55 |
| 644 | Rofecoxib | H56 |
| 645 | Rofecoxib | H57 |
| 646 | Rofecoxib | H58 |
| 647 | Rofecoxib | H59 |
| 648 | Rofecoxib | H60 |
| 649 | Rofecoxib | H61 |
| 650 | Rofecoxib | H62 |
| 651 | Rofecoxib | H63 |
| 652 | Rofecoxib | H64 |
| 653 | Rofecoxib | H65 |
| 654 | Rofecoxib | H66 |
| 655 | Rofecoxib | H67 |
| 656 | Rofecoxib | H68 |
| 657 | Rofecoxib | H69 |
| 658 | Rofecoxib | H70 |
| 659 | Rofecoxib | H71 |
| 660 | Rofecoxib | H72 |
| 661 | Rofecoxib | H73 |
| 662 | Rofecoxib | H74 |
| 663 | Rofecoxib | H75 |
| 664 | Rofecoxib | H76 |
| 665 | Rofecoxib | H77 |
| 666 | Rofecoxib | H78 |
| 667 | Rofecoxib | H79 |
| 668 | Rofecoxib | H80 |
| 669 | Rofecoxib | H81 |
| 670 | Rofecoxib | H82 |
| 671 | Rofecoxib | H83 |
| 672 | Rofecoxib | H84 |
| 673 | Meloxicam | H1 |
| 674 | Meloxicam | H2 |
| 675 | Meloxicam | H3 |
| 676 | Meloxicam | H4 |
| 677 | Meloxicam | H5 |
| 678 | Meloxicam | H6 |
| 679 | Meloxicam | H7 |
| 680 | Meloxicam | H8 |
| 681 | Meloxicam | H9 |
| 682 | Meloxicam | H10 |
| 683 | Meloxicam | H11 |
| 684 | Meloxicam | H12 |
| 685 | Meloxicam | H13 |
| 686 | Meloxicam | H14 |
| 687 | Meloxicam | H15 |
| 688 | Meloxicam | H16 |
| 689 | Meloxicam | H17 |
| 690 | Meloxicam | H18 |
| 691 | Meloxicam | H19 |
| 692 | Meloxicam | H20 |
| 693 | Meloxicam | H21 |
| 694 | Meloxicam | H22 |
| 695 | Meloxicam | H23 |
| 696 | Meloxicam | H24 |
| 697 | Meloxicam | H25 |
| 698 | Meloxicam | H26 |
| 699 | Meloxicam | H27 |
| 700 | Meloxicam | H28 |
| 701 | Meloxicam | H29 |
| 702 | Meloxicam | H30 |
| 703 | Meloxicam | H31 |
| 704 | Meloxicam | H32 |
| 705 | Meloxicam | H33 |
| 706 | Meloxicam | H34 |
| 707 | Meloxicam | H35 |
| 708 | Meloxicam | H36 |
| 709 | Meloxicam | H37 |
| 710 | Meloxicam | H38 |
| 711 | Meloxicam | H39 |
| 712 | Meloxicam | H40 |
| 713 | Meloxicam | H41 |
| 714 | Meloxicam | H42 |
| 715 | Meloxicam | H43 |
| 716 | Meloxicam | H44 |
| 717 | Meloxicam | H45 |
| 718 | Meloxicam | H46 |
| 719 | Meloxicam | H47 |
| 720 | Meloxicam | H48 |
| 721 | Meloxicam | H49 |
| 722 | Meloxicam | H50 |
| 723 | Meloxicam | H51 |
| 724 | Meloxicam | H52 |
| 725 | Meloxicam | H53 |
| 726 | Meloxicam | H54 |
| 727 | Meloxicam | H55 |
| 728 | Meloxicam | H56 |
| 729 | Meloxicam | H57 |
| 730 | Meloxicam | H58 |
| 731 | Meloxicam | H59 |
| 732 | Meloxicam | H60 |
| 733 | Meloxicam | H61 |
| 734 | Meloxicam | H62 |
| 735 | Meloxicam | H63 |
| 736 | Meloxicam | H64 |
| 737 | Meloxicam | H65 |
| 738 | Meloxicam | H66 |
| 739 | Meloxicam | H67 |
| 740 | Meloxicam | H68 |
| 741 | Meloxicam | H69 |
| 742 | Meloxicam | H70 |
| 743 | Meloxicam | H71 |
| 744 | Meloxicam | H72 |
| 745 | Meloxicam | H73 |
| 746 | Meloxicam | H74 |
| 747 | Meloxicam | H75 |
| 748 | Meloxicam | H76 |
| 749 | Meloxicam | H77 |
| 750 | Meloxicam | H78 |
| 751 | Meloxicam | H79 |
| 752 | Meloxicam | H80 |
| 753 | Meloxicam | H81 |
| 754 | Meloxicam | H82 |
| 755 | Meloxicam | H83 |
| 756 | Meloxicam | H84 |
| 757 | A chromene | H1 |
| | Cox-2 inhibitor | |
| 758 | A chromene | H2 |
| | Cox-2 inhibitor | |
| 759 | A chromene | H3 |
| | Cox-2 inhibitor | |
| 760 | A chromene | H4 |
| | Cox-2 inhibitor | |
| 761 | A chromene | H5 |
| | Cox-2 inhibitor | |
| 762 | A chromene | H6 |
| | Cox-2 inhibitor | |
| 763 | A chromene | H7 |
| | Cox-2 inhibitor | |
| 764 | A chromene | H8 |
| | Cox-2 inhibitor | |
| 765 | A chromene | H9 |
| | Cox-2 inhibitor | |
| 766 | A chromene | H10 |
| | Cox-2 inhibitor | |
| 767 | A chromene | H11 |
| | Cox-2 inhibitor | |
| 768 | A chromene | H12 |
| | Cox-2 inhibitor | |
| 769 | A chromene | H13 |
| | Cox-2 inhibitor | |
| 770 | A chromene | H14 |
| | Cox-2 inhibitor | |
| 771 | A chromene | H15 |
| | Cox-2 inhibitor | |
| 772 | A chromene | H16 |
| | Cox-2 inhibitor | |
| 773 | A chromene | H17 |
| | Cox-2 inhibitor | |
| 774 | A chromene | H18 |
| | Cox-2 inhibitor | |
| 775 | A chromene | H19 |
| | Cox-2 inhibitor | |
| 776 | A chromene | H20 |
| | Cox-2 inhibitor | |
| 777 | A chromene | H21 |
| | Cox-2 inhibitor | |
| 778 | A chromene | H22 |
| | Cox-2 inhibitor | |
| 779 | A chromene | H23 |
| | Cox-2 inhibitor | |
| 780 | A chromene | H24 |
| | Cox-2 inhibitor | |
| 781 | A chromene | H25 |
| | Cox-2 inhibitor | |
| 782 | A chromene | H26 |
| | Cox-2 inhibitor | |
| 783 | A chromene | H27 |
| | Cox-2 inhibitor | |
| 784 | A chromene | H28 |
| | Cox-2 inhibitor | |
| 785 | A chromene | H29 |
| | Cox-2 inhibitor | |
| 786 | A chromene | H30 |
| | Cox-2 inhibitor | |
| 787 | A chromene | H31 |
| | Cox-2 inhibitor | |
| 788 | A chromene | H32 |
| | Cox-2 inhibitor | |
| 789 | A chromene | H33 |
| | Cox-2 inhibitor | |
| 790 | A chromene | H34 |
| | Cox-2 inhibitor | |
| 791 | A chromene | H35 |
| | Cox-2 inhibitor | |
| 792 | A chromene | H36 |
| | Cox-2 inhibitor | |
| 793 | A chromene | H37 |
| | Cox-2 inhibitor | |
| 794 | A chromene | H38 |
| | Cox-2 inhibitor | |
| 795 | A chromene | H39 |
| | Cox-2 inhibitor | |
| 796 | A chromene | H40 |
| | Cox-2 inhibitor | |
| 797 | A chromene | H41 |
| | Cox-2 inhibitor | |
| 798 | A chromene | H42 |
| | Cox-2 inhibitor | |
| 799 | A chromene | H43 |
| | Cox-2 inhibitor | |
| 800 | A chromene | H44 |
| | Cox-2 inhibitor | |
| 801 | A chromene | H45 |
| | Cox-2 inhibitor | |
| 802 | A chromene | H46 |
| | Cox-2 inhibitor | |
| 803 | A chromene | H47 |
| | Cox-2 inhibitor | |
| 804 | A chromene | H48 |
| | Cox-2 inhibitor | |
| 805 | A chromene | H49 |
| | Cox-2 inhibitor | |
| 806 | A chromene | H50 |
| | Cox-2 inhibitor | |
| 807 | A chromene | H51 |
| | Cox-2 inhibitor | |
| 808 | A chromene | H52 |
| | Cox-2 inhibitor | |
| 809 | A chromene | H53 |
| | Cox-2 inhibitor | |
| 810 | A chromene | H54 |
| | Cox-2 inhibitor | |
| 811 | A chromene | H55 |
| | Cox-2 inhibitor | |
| 812 | A chromene | H56 |
| | Cox-2 inhibitor | |
| 813 | A chromene | H57 |
| | Cox-2 inhibitor | |
| 814 | A chromene | H58 |
| | Cox-2 inhibitor | |
| 815 | A chromene | H59 |
| | Cox-2 inhibitor | |
| 816 | A chromene | H60 |
| | Cox-2 inhibitor | |
| 817 | A chromene | H61 |
| | Cox-2 inhibitor | |
| 818 | A chromene | H62 |
| | Cox-2 inhibitor | |
| 819 | A chromene | H63 |
| | Cox-2 inhibitor | |
| 820 | A chromene | H64 |
| | Cox-2 inhibitor | |
| 821 | A chromene | H65 |
| | Cox-2 inhibitor | |
| 822 | A chromene | H66 |
| | Cox-2 inhibitor | |
| 823 | A chromene | H67 |
| | Cox-2 inhibitor | |
| 824 | A chromene | H68 |
| | Cox-2 inhibitor | |
| 825 | A chromene | H69 |
| | Cox-2 inhibitor | |
| 826 | A chromene | H70 |
| | Cox-2 inhibitor | |
| 827 | A chromene | H71 |
| | Cox-2 inhibitor | |
| 828 | A chromene | H72 |
| | Cox-2 inhibitor | |
| 829 | A chromene | H73 |
| | Cox-2 inhibitor | |
| 830 | A chromene | H74 |
| | Cox-2 inhibitor | |
| 831 | A chromene | H75 |
| | Cox-2 inhibitor | |
| 832 | A chromene | H76 |
| | Cox-2 inhibitor | |
| 833 | A chromene | H77 |
| | Cox-2 inhibitor | |
| 834 | A chromene | H78 |
| | Cox-2 inhibitor | |
| 835 | A chromene | H79 |
| | Cox-2 inhibitor | |
| 836 | A chromene | H80 |
| | Cox-2 inhibitor | |
| 837 | A chromene | H81 |
| | Cox-2 inhibitor | |
| 838 | A chromene | H82 |
| | Cox-2 inhibitor | |
| 839 | A chromene | H83 |
| | Cox-2 inhibitor | |
| 840 | A chromene | H84 |
| | Cox-2 inhibitor | |

Biological Assays

Evaluation of Cox-1 and Cox-2 Activity In Vitro

The Cox-2 inhibiting agents of this invention exhibit Cox-2 inhibition in vitro. The Cox-2 inhibition activity of the compounds illustrated in the example above are determined by the following methods. The Cox-2 inhibition activity of the other Cox-2 inhibitors of the present invention may also be determined by the following methods.

Preparation of Recombinant Cox Baculoviruses:

Recombinant Cox-1 and Cox-2 are prepared as described by Gierse et al., [J. Biochem., 305, 479-84 (1995)]. A 2.0 kb fragment containing the coding region of either human or murine Cox-1 or human or murine Cox-2 is cloned into a BamH1 site of the baculovirus transfer vector pVL1393 (Invitrogen) to generate the baculovirus transfer vectors for Cox-1 and Cox-2 in a manner similar to the method of D. R. O'Reilly et al. (Baculovirus Expression Vectors: A Laboratory Manual (1992)). Recombinant baculoviruses are isolated by transfecting 4 μg of baculovirus transfer vector DNA into SF9 insect cells (2×10⁸) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses are purified by three rounds of plaque purification and high titer (107-108 pfu/mL) stocks of virus are prepared. For large scale production, SF9 insect cells are infected in 10 liter fermentors (0.5×10⁶/mL) with the recombinant baculovirus stock such that the multiplicity of infection is 0.1. After 72 hours the cells are centrifuged and the cell pellet is homogenized in Tris/Sucrose (50 mM: 25%, pH 8.0) containing 1% 3-[(3-cholamidopropyl)-dimethylammonio]-1-propanesulfonate (CHAPS). The homogenate is centrifuged at 10,000×G for 30 minutes, and the resultant supernatant is stored at −80° C. before being assayed for Cox activity.

Assay for Cox-1 and Cox-2 Activity:

Cox activity is assayed as PGE2 formed/μg protein/time using an ELISA to detect the prostaglandin released. CHAPS-solubilized insect cell membranes containing the appropriate Cox enzyme are incubated in a potassium phosphate buffer (50 mM, pH 8.0) containing epinephrine, phenol, and heme with the addition of arachidonic acid (10 μM). Compounds are pre-incubated with the enzyme for 10-20 minutes prior to the addition of arachidonic acid. Any reaction between the arachidonic acid and the enzyme is stopped after ten minutes at 37° C./room temperature by transferring 40 μl of reaction mix into 160 μl ELISA buffer and 25 μM indomethacin. The PGE2 formed is measured by standard ELISA technology (Cayman Chemical).

Fast Assay for Cox-1 and Cox-2 Activity:

Cox activity is assayed as PGE2 formed/μg protein/time using an ELISA to detect the prostaglandin released. CHAPS-solubilized insect cell membranes containing the appropriate Cox enzyme are incubated in a potassium phosphate buffer (0.05 M Potassium phosphate, pH 7.5, 2 μM phenol, 1 μM heme, 300 μM epinephrine) with the addition of 20 μl of 100 μM arachidonic acid (10 μM). Compounds are pre-incubated with the enzyme for 10 minutes at 25° C. prior to the addition of arachidonic acid. Any reaction between the arachidonic acid and the enzyme is stopped after two minutes at 37° C./room temperature by transferring 40 μl of reaction mix into 160 μl ELISA buffer and 25 μM indomethacin. The PGE2 formed is measured by standard ELISA technology (Cayman Chemical).

Evaluation of 5-HT_{1A} Activity In Vitro

5-HT_{1A} Receptor Binding Profile:

Compounds are tested for binding to cloned human 5-HT_{1A} receptors stably transfected into CHO cells using [³H]8-OH-DPAT as the 5-HT_{1A} radioligand (according to general procedure described in J. Dunlop et al., J. Pharmacol. Tox. Methods, 40:47-55 (1998)).

5-HT_{1A} Functional Activity Assay:

A clonal cell line stably transfected with the human 5-HT_{1A} receptor is utilized to determine the intrinsic activity of compounds (according to the general procedure described in J. Dunlop et al., J. Pharmacol. Tox. Methods, 40:47-55 (1998)). Compounds of the present invention are tested for their efficacy in antagonizing the ability of 10 nM 8-OH-DPAT to inhibit forskolin stimulated cAMP production in a concentration-related fashion.

Biological Evaluation:

A combination therapy of a Cox-2 inhibiting agent and a 5-HT_{1A} receptor modulator for the treatment or prevention of pain, inflammation, or an inflammation-related disorder in a mammal can be evaluated as described in the following tests.

Rat Focal Stroke Model:

The efficacy of the compositions of the present invention for the prevention and treatment of focal stroke in rats can be determined according to the method described in Nogawa, S. et al., J. of Neuroscience, 17(8):2748-2755 (1997).

Induction and Assessment of Collagen Induced Arthritis in Mice:

Arthritis is induced in 8-12 week old male DBA/1 mice by injection of 50 mg of chick type II collagen (CII) in complete Freunds adjuvant (Sigma) on day 0 at the base of the tail as previously described [J. Stuart, Annual Rev. Immunol., 2:199 (1984)]. Compounds are prepared as a suspension in 0.5% methylcellulose (Sigma, St. Louis, Mo.), 0.025% Tween 20 (Sigma). The Cox-2 inhibitors and the 5-HT_{1A} receptor modulator are administered alone or a Cox-2 inhibitor and 5-HT_{1A} receptor modulator in combination. The compounds are administered in non-arthritic animals by gavage in a volume of 0.1 ml beginning on day 20 post collagen injection and continuing daily until final evaluation on day 55.

Animals are boosted on day 21 with 50 mg of collagen (CII) in incomplete Freunds adjuvant. The animals are subsequently evaluated several times each week for incidence and severity of arthritis until approximately day 56. Any animal with paw redness or swelling is counted as arthritic. Scoring of severity is carried out using a score of 0-3 for each paw (maximal score of 12/mouse) as previously described [P. Wooley, et al., Trans. Proc., 15:180 (1983)]. The animals are measured for incidence of arthritis and severity in the animals where arthritis is observed. The incidence of arthritis is determined at a gross level by observing the swelling or redness in the paw or digits. Severity is measured with the following guidelines. Briefly, animals displaying four normal paws, i.e., no redness or swelling are scored 0. Any redness or swelling of digits or the paw is scored as 1. Gross swelling of the whole paw or deformity is scored as 2. Ankylosis of joints is scored as 3.

Histological Examination of Paws:

In order to verify the gross determination of a non-arthritic animal, a histological examination is performed. Paws from animals sacrificed at the end of the experiment are removed, fixed and decalcified as previously described [R. Jonsson, J. Immunol. Methods, 88:109 (1986)]. Samples are paraffin embedded, sectioned, and stained with hernatoxylin and eosin by standard methods. Stained sections are examined for cellular infiltrates, synovial hyperplasia, and bone and cartilage erosion.

All references cited in this specification, including without limitation all papers, publications, presentations, texts, reports, manuscripts, brochures, books, internet postings, journal articles, periodicals, and the like, are hereby incorporated by reference. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the methods, combinations and compositions of the present invention as indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A composition comprising a Cox-2 inhibitor and a 5-HT_{1A} receptor modulator.

2. The composition according to claim 1, wherein the amount of the Cox-2 inhibitor and the amount of the 5-HT_{1A} receptor modulator together comprise a therapeutically effective amount for the treatment or prevention of pain, inflammation or an inflammation-related disorder.

3. The composition according to claim 1, wherein the Cox-2 inhibitor comprises a non-steroidal anti-inflammatory drug.

4. The composition according to claim 3, wherein the Cox-2 inhibitor is selected from the group consisting of ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam, and 2-fluoro-a-methyl[1,1′-biphenyl]-4-acetic acid, 4-(nitrooxy)butyl ester.

5. The composition according to claim 4, wherein the Cox-2 inhibitor comprises 2-fluoro-a-methyl[1,1′-biphenyl]-4-acetic acid, 4-(nitrooxy)butyl ester.

6. The composition according to claim 1, wherein the Cox-2 inhibitor comprises a Cox-2 selective inhibitor.

7. The composition according to claim 6, wherein the Cox-2 selective inhibitor comprises at least one compound selected from the group consisting of celecoxib, deracoxib, parecoxib, valdecoxib, rofecoxib, lumiracoxib, etoricoxib, meloxicam, and mixtures and prodrugs thereof.

8. The composition according to claim 7, wherein the Cox-2 selective inhibitor comprises at least one compound selected from the group consisting of celecoxib, valdecoxib, rofecoxib, and mixtures thereof.

9. The composition according to claim 1, wherein the Cox-2 selective inhibitor comprises a chromene Cox-2 selective inhibitor.

10. The composition according to claim 1, wherein the 5-HT_{1A} receptor modulator comprises at least one compound selected from the group consisting of:
(R)-N-(1,3-benzodioxol-5-ylmethyl)-1,2,3,4-tetrahydro-[1]benzothieno[2,3-c]pyridine-3-carboxamide (AP-521), 1-[3-[4-(3-chlorophenyl)-1-piperazinyl]propyl]-3,4-dihydro-5-methoxy-2(1H)-quinolinone (OPC-14523), 2-[4-[4-(7-chloro-2,3-dihydro-1,4-benzodioxin-5-yl)-1-piperazinyl]butyl]-1,2-benzisothiazol-3(2H)-one-1,1-dioxide (DU-125530), 7-(4-methyl-1-piperazinyl)-2(3H)benzoxazolone, monohydrochloride (SLV-308), adatanserin, alnespirone, binospirone, buspirone, DU-127090, E-2101, eptapirone, flibanserin, gepirone, ipsapirone, lesopitron, N-[2-[4-(2-methoxyphenyl)-1-piperazinyl]ethyl]-N-2-pyridinyl-cyclohexanecarboxamidetrihydrochloride (WAY-100635), N-[3-(1,3-benzodioxol-5-yloxy)propyl]-2,3-dihydro-(2S)-1,4-benzodioxin-2-methanaminehydrochloride (MKC-242), repinotan, robalzotan, sarizotan, SLV-319, SUN-N4057, tandospirone, vilazodone, VML-670, xaliproden, ziprasidone, 6-hydroxy-buspirone, pyrazolidine derivative, heteroaryloxyethylamines, 5-hydoxytryptamine, 5-methoxytryptamine, buspirone, 8-hydroxy-2-(di-n-propylamino)tetralin (8-OH-DPAT), ipsaspirone, gepirone, SM23997, lysergic acid diethylamide, agonistic antibodies, piperazine derivatives, 8-(2-aminoalkoxy)fluorochroman derivatives, abeo-ergoline derivatives, A-74283, AP-159, AZ 16596, 2-[4-(2-methoxyphenyl)piperazin-1-yl]methyl] octahydroimidazo [1,5-a]pyridine-1,3-dione (B 20991), BMS 181100 (BMY 42569), BMS 181970,1-methyl-4-[7-(4-chlorophenyl)methylaminocarbonyl] napththyl-piperazine (CP291952), (omega-piperazinylalkoxy) alkylenedioxybenzene (BP 554), E 5165, E 6265, ebalzotan, eltoprazine, F 11440, F 13714, flesinoxan, 2-[4-(3-phenylpyrrolidin-1-yl)butyl]-1,2-benzisothiazol-3(2H)-one 1,1-dioxide (LB 50016), LY 41, (+/−)-4-substituted-amino-6-substituted-1,3,4,5-tetrahydrobenz[c,d]inoles (LY 228729), LY 228730, LY 274600, LY 274601, LY 293284, 6-heterocyclyl-4-amino-1,3,4,5-tetrahydrobenz CD indoles (LY 297996), isoxazole derivatives (LY 315535), hetero-oxy alkanamines (LY 333068), LY 426965, LY 433221, MDL 72832, MDL 73975, NDL 249, nerisopam, Org 1301, 2-(2-oxo-hexahydropyrimidin-1-yl)propylaminomethyl-benzopyran (R137696), RU 24969, 1-[[5-[[4-substituted-1-pipe razinyl]methyl]-pyrrol-2-yl or furan-2-yl]methyl-2-piperidinones (RWJ 25730), S 14489, S 14506, S 14671, S 15535, S 15931, 8-[4-[N-(5-Acetyl-3,4-dihydro-2H-1-benzopyran-3-yl)-Npropylamino]butyl]-8-azaspiro [4.5]decane-7,9-dione (S 23751), SDZ 216-525, SEP 109235, SR59026, Sunepitron, UH 301, WAY 100135, WAY 100802, [(3-chloro-4-fluoro-phenyl)-[4-fluoro-4-{[(5-methyl-pyridin-2-ylmethyl)amino]-methyl)piperidin-1-yl]-methadone] (F 13640), zalospirone, a pharmaceutically acceptable salt of any one of the compounds, and mixtures of two or more of the compounds.

11. The composition according to claim 1, wherein the 5-HT_{1A} receptor modulator comprises at least one compound that is selected from the group consisting of buspirone, gepirone, repinotan, tandospirone, xaliproden, ziprasidone, and mixtures thereof.

12. A method for the treatment or prevention of pain, inflammation, or inflammation-related disorder in a subject in need thereof, comprising administering to the subject a Cox-2 inhibitor and a 5-HT_{1A} receptor modulator.

13. The method according to claim 12, wherein the amount of the Cox-2 inhibitor and the amount of the 5-HT_{1A} receptor modulator together comprise a therapeutically effective amount for the treatment or prevention of pain, inflammation or an inflammation-related disorder in the subject.

14. The method according to claim 12, wherein the Cox-2 inhibitor comprises at least one non-steroidal anti-inflammatory drug that is selected from the group consisting of ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam, and 2-fluoro-a-methyl[1,1′-biphenyl]-4-acetic acid, 4-(nitrooxy)butyl ester.

15. The method according to claim 12, wherein the Cox-2 inhibitor comprises a Cox-2 selective inhibitor.

16. The method according to claim 12, wherein the Cox-2 selective inhibitor comprises at least one compound that is selected from the group consisting of celecoxib, deracoxib, valdecoxib, parecoxib, lumiracoxib, rofecoxib, etoricoxib, meloxicam, and mixtures and prodrugs thereof.

17. The method according to claim 12, wherein the Cox-2 selective inhibitor comprises at least one compound selected from the group consisting of celecoxib, parecoxib, rofecoxib, and mixtures thereof.

18. The method according to claim 12, wherein the 5-HT_{1A} receptor modulator comprises at least one compound selected from the group consisting of:
(R)-N-(1,3-benzodioxol-5-ylmethyl)-1,2,3,4-tetrahydro-[1]benzothieno[2,3-c]pyridine-3-carboxamide (AP-521), 1-[3-[4-(3-chlorophenyl)-1-piperazinyl]propyl]-3,4-dihydro-5-methoxy-2(1H)-quinolinone (OPC-14523), 2-[4-[4-(7-chloro-2,3-dihydro-1,4-benzodioxin-5-yl)-1-piperazinyl]butyl]-1,2-benzisothiazol-3(2H)-one-1,1-dioxide (DU-125530), 7-(4-methyl-1-piperazinyl)-2(3H)benzoxazolone, monohydrochloride (SLV-308), adatanserin, alnespirone, binospirone, buspirone, DU-127090, E-2101, eptapirone, flibanserin, gepirone, ipsapirone, lesopitron, N-[2-[4-(2-methoxyphenyl)-1-piperazinyl]ethyl]-N-2-pyridinyl-cyclohexanecarboxamidetrihydrochloride (WAY-100635), N-[3-(1,3-benzodioxol-5-yloxy)propyl]-2,3-dihydro-(2S)-1,4-benzodioxin-2-methanaminehydrochloride (MKC-242), repinotan, robalzotan, sarizotan, SLV-319, SUN-N4057, tandospirone, vilazodone, VML-670, xaliproden, ziprasidone, 6-hydroxy-buspirone, pyrazolidine derivative, heteroaryloxyethylamines, 5-hydoxytryptamine, 5-methoxytryptamine, buspirone, 8-hydroxy-2-(di-n-propylamino)tetralin (8-OH-DPAT), ipsaspirone, gepirone, SM23997, lysergic acid diethylamide, agonistic antibodies, piperazine derivatives, 8-(2-aminoalkoxy)fluorochroman derivatives, abeo-ergoline derivatives, A-74283, AP-159, AZ 16596, 2-[4-(2-methoxyphenyl)piperazin-1-yl]methyl] octahydroimidazo [1,5-a]pyridine-1,3-dione (B 20991), BMS 181100 (BMY 42569), BMS 181970,1-methyl-4-[7-(4-chlorophenyl)methylaminocarbonyl] napththyl-piperazine (CP291952), (omega-piperazinylalkoxy) alkylenedioxybenzene (BP 554), E 5165, E 6265, ebalzotan, eltoprazine, F 11440, F 13714, flesinoxan, 2-[4-(3-phenylpyrrolidin-1-yl)butyl] -1,2-benzisothiazol-3(2H)-one 1,1-dioxide (LB 50016), LY 41, (+/−)-4-Substituted-amino-6-substituted-1,3,4,5-tetrahydrobenz[c,d]inoles (LY 228729), LY 228730, LY 274600, LY 274601, LY 293284, 6-heterocyclyl-4-amino-1,3,4,5-tetrahydrobenz CD indoles (LY 297996), isoxazole derivatives (LY 315535), hetero-oxy alkanamines, (LY 333068), LY 426965, LY 433221, MDL 72832, MDL 73975, NDL 249, nerisopam, Org 1301, 2-(2-oxo-hexahydropyrim idin-1-yl)propylaminomethyl-benzopyran (R137696), RU 24969,1-[[5-[[4-substituted-1-piperazinyl]methyl]-pyrrol-2-yl or furan-2-yl]methyl-2-piperidinones (RWJ 25730), S 14489, S 14506, S 14671, S 15535, S 15931, 8-[4-[N-(5-Acetyl-3,4-dihydro-2H-1-benzopyran-3-yl)-Npropylamino]butyl]-8-azaspiro
decane-7,9-dione (S 23751), SDZ 216-525, SEP 109235, SR59026, Sunepitron, UH 301, WAY 100135, WAY 100802, [(3-chloro-4-fluoro-phenyl)-[4-fluoro-4-{[(5-methyl-pyridin-2-ylmethyl)amino]-methyl)piperidin-1-yl]-methadone] (F 13640), zalospirone, and mixtures thereof.
or a pharmaceutically acceptable salt of the compound.

19. The method according to claim 12, wherein the 5-HT_{1A} receptor modulator comprises at least one compound that is selected from the group consisting of of buspirone, gepirone, repinotan, tandospirone, xaliproden, ziprasidone, and mixtures thereof.

20. The method according to claim 12, wherein the inflammation-related disorder is selected from the group consisting of central nervous system disorder, cognitive dysfunction, and glaucoma.

21. The method according to claim 20, wherein the central nervous system disorder is a disorder associated with stroke (ischemic or hemorrhagic) or ischemic brain injury.

22. The method according to claim 12, wherein the pain, inflammation or inflammation related disorder is selected from the group consisting of adjustment disorders, anxiety (mixed anxiety), mood (depressed), conduct disturbance, mixed anxiety and mood (conduct), addictive disorders, alcohol abuse, intoxication disorders, nicotine abuse, psychoactive substances abuse, substance disorder, withdrawal syndromes, acute trauma, age associated mental disorders, learning disorders, Alzheimer's disease, agitation disorders, agitation in Alzheimer's disease, agitation in the elderly, aggressive behavior, aggressive behavior in Alzheimers disease, amyloidosis, aging/senile amyloidosis, hereditary amyloidosis, immunocyte derived amyloidosis, lichen amyloidosis, primary amyloidosis, reactive systemic amyloidosis, secondary amyloidosis, senile amyloidosis (Alzheimer's disease), amyotrophy & amyotripic lateral scherosis (ALS), ALS, anorexia nervosa, anxiety disorders, generalized anxiety disorder (GAD), social phobias, stress related diseases, apathy, attention deficit disorder (ADD), attention deficit hyperactivity disorder (ADHD), autism, auto immune disorders, lupus erythematosis, multiple sclerosis, behavioral disturbances, agitation plus diminished cognition, bipolar I disorder, bipolar II disorder, bulimia nervosa, cardiovascular disorders, blood pressure modification, hypertension, hypotension, heart rate modification, chemotherapy-induced vomiting, chronic fatigue immune disorders (CFIDS), chronic fatigue syndrome (CFS), cognitive dysfunction, cortical dementias, mild cognitive impairment (MCI), Lewy Body dementia, vascular dementia, neurodegeneration, cognitive dysfunction resulting from stroke, ischemia, trauma, or surgical procedures, including coronary artery bypass surgery, cognition enhancement, conduct disorder, cyclothymia, delusional disorder, depression, adolescent depression, depression in Alzheimer's disease, general depression, minor depression, depression in Parkinson's disease, depression in diabetic neuropathy, dissociative disorders, developmental disorders, learning disabilities, language disorders, mental retardation, dementia, dementias associated with aging, illness, neurodegeneration and dyskensia, dysthymia, dystonia, eating disorders associated with anorexia nervosa, bulimia nervosa, obesity, epilepsy, or fibromyalgia syndrome (FMS), gastrointestinal disorders, irritable bowel syndrome, psychogenic effects and stress-related; growth retardation effects, endocrine, psychosocial and stress-related retardation, heart rate modification, Huntington's chorea, hypertension, immune system disorders, immune system depression, impulse control disorders, incontinence, infectious neuropathy, AIDS, carpal tunnel syndrome, dementia, irritable bowel syndrome (IBS), constipative IBS, diarrhea-predominant IBS, inflammatory bowel disease (IBD), constipation-predominant IBD, diarrhea-predominant IBD, mixed states IBD, inhalation disorder, lactation inhibition, metabolic & chromosomal disorders, galactosemia phenylketonuria, fatty acid disorder, infantile nephropathic cystinosis, orthithrotranscarbamylase porphyria, migrane, mood disorders, a typical depression, bipolar disorder (including pychotic features), major depressive disorder, mania, seasonal affective disorder, movement disorders, athetosis, chorea, dyskinesia, dystonia, restless leg syndrome (RLS), tremor plus periodic limb movement (PLM), periodic limb movements of sleep (PLMS), Parkinson's disease, PLM, PLMS, progressive supranuclear palsy, stereotypy (various), torticollis, tic disorders, tremor; multisystemic atrophy (MSA), multiple sclerosis, neuroendocrine system disorders, neurodegenerative disorders, amyotrophy, amyotrophy diabetics, amyotrophic lateral sclerosis (ALS), Parkinson's disease, neurological disorders, neuropathy, diabetic neuropathy, peripheral neuropathy, neuroprotective effects for ischemic brain injury, neuroprotective effects for myocardial infarction, neuroprotective effects for spinal cord injury, neuroprotective effects for traumatic brain injury, neuroprotective effects for obesity, obsessive compulsive disorder (OCD), oncology related disorders, behavior abnormalities resulting from tumors or treatments, oppositional defiant disorder, pain disorders, acute pain, chronic pain, cluster headache, dysmenorrhea, labor pain, migraine pain, neuropathic pain, AIDs-related pain, AIDS-associated dementia, cancer-related pain, chemotherapeutic-induced pain, diabetic pain, post-herpetic neuralgia, radiation-induced pain, osteoarthritis flare, phantom limb pain, surgical pain, post-surgical pain, incisional pain, psychic pain, regional pain, abdominal pain, chronic back pain, complex-regional pain disorder, dental, face and mouth pain, head pain, lower back and peripheral pain, rheumatoid arthritis pain, starting pain, systematic pain, connective tissue pain, musculoskeletal pain, nervous system pain, urogenital pain, uterine contraction pain, panic disorder, agoraphobia, peripheral neuropathy, personality disorders, phobias (simple), phobias of animals, phobias of closed spaces (claustrophobia), phobias of heights (acrophobia), phobias of public places (agoraphobia), social phobias, phobia of public eating, phobia of public embarrassment, phobia of public performance/speaking and using public lavatories, poop out syndrome, SSRI, post-traumatic stress disorder, progressive supranuclear palsy (PSP), prolactin plasma level disorders, psychotic disorders, brief psychosis, long duration psychosis, psychosis due to medical condition, restless leg syndrome (RLS), schizophrenias, delusional (paranoid) disorder, schizoaffective disorders, schizophreniform disorders, seasonal affective disorder, seizure disorders, epilepsy (partial), epilepsy (generalized), sexual dysfunction, sleep disorders, apnea, parasomnias, insomnia, narcolepsy, obstructive sleep disorder, disorders of circadian rhythm, enuresis, initiation, or maintenance, social phobias, social anxiety disorder, somatoform disorders, conversion, body, dysmorphic somatoform disorder, fibromyalgia syndrome (FMS), hypochondriasis, NOS, somatization, undifferentiated somatoform disorder, developmental disorders, stress disorders, acute stress disorder, chronic stress disorder, incontinence, spectrum disorders, stroke, suicidal behavior, thyroid stimulating hormone disorders (TSH), Tourette's syndrome, tooth-germ morphogenesis disorders, thermoregulation disorders, TSH modulating agent disorders, tic disorders, trauma, acute trauma, head trauma, vasospasms, vasoreactive headaches and violent behavior.

23. The method of claim 12, wherein the subject is a mammal.

24. A pharmaceutical composition for the treatment or prevention of pain, inflammation, or inflammation-related disorder, the pharmaceutical composition comprising a Cox-2 inhibitor, a 5-HT_{1A} receptor modulator, and a pharmaceutically-acceptable excipient.

25. A kit that is suitable for use in the treatment or prevention of pain, inflammation, or inflammation-related disorder wherein the kit comprises a first dosage form comprising a Cox-2 inhibitor and a second dosage form comprising a 5-HT_{1A} receptor modulator, in quantities which comprise a therapeutically effective amount of the compounds for the treatment, prevention or inhibition of pain, inflammation, or an inflammation-related disorder.

26. A method for the prevention or treatment of a neurologic disorder involving neurodegeneration in a subject that is in need of such prevention or treatment, the method comprising administering to the subject a Cox-2 inhibitor and a 5-HT_{1A} receptor modulator.
